(19) United States
(12) Patent Application Publication
(10) Pub. No.: US 2022/0280423 A1
(43) Pub. Date: Sep. 8, 2022

(54) GLYMPHATIC DELIVERY BY MANIPULATING PLASMA OSMOLARITY

(71) Applicant: University of Rochester, New York, NY (US)

(72) Inventors: Maiken Nedergaard, Rochester, NY (US); Benjamin Plog, New York, NY (US); Humberto Mestre, New York, NY (US); Tuomas O. Lilius, Helsinki (FI); Andreas I. Jensen, Virum (DK); Steven A. Goldman, Webster, NY (US)

(73) Assignee: University of Rochester, New York, NY (US)

(21) Appl. No.: 17/699,953

(22) Filed: Mar. 21, 2022

Related U.S. Application Data
(63) Continuation-in-part of application No. 17/280,606, filed on Mar. 26, 2021.
(60) Provisional application No. 62/741,295, filed on Oct. 4, 2018.

(51) Int. Cl. A61K 9/00; A61K 47/02; C07K 16/18; A61K 9/51

(52) U.S. Cl. CPC A61K 9/0085; A61K 47/02; C07K 16/18; A61K 9/5115; A61K 9/5146

(57) ABSTRACT

This invention relates to improving delivery of agents (e.g., one or more nanoparticles) to the central nervous system.

### CROSS REFERENCE TO RELATED APPLICATION

This application is a continuation-in-part application of U.S. Patent Application Ser. No. 17/280,606, which is a U.S. National Phase of International Application No.: PCT/US2019/053808, filed Sep. 30, 2019, which claims priority to U.S. Provisional Application No. 62/741,295 filed on Oct. 4, 2018. The contents of the applications are incorporated herein by reference in their entireties.

### GOVERNMENT INTERESTS

This invention was made with government support under R01NS100366 and RF1AG057575 awarded by National Institutes of Health and under W81XWH-16-1-0555 awarded by the Office of the Assistant Secretary of Defense for Health Affairs. The government has certain rights in the invention.

### FIELD OF THE INVENTION

This invention relates to improving delivery of agents to the central nervous system.

### BACKGROUND OF THE INVENTION

Various therapeutic agents have been developed for treating central nervous system (CNS) diseases. However, delivery therapeutic agents to the brain is severely limited by the largely impermeable blood-brain barrier (BBB) and poor penetration of the therapeutic agents to the brain. Improving the delivery of drugs to the CNS is a considerable clinical challenge (4, 19, 55), especially in the settings of immunotherapy. For example, therapies based on monoclonal antibodies (mAb) are currently being developed for CNS diseases such as Alzheimer's disease (AD) (1), Parkinson's disease (2), amyotrophic lateral sclerosis (ALS) (3), frontotemporal lobar dementia (FTLD)(4), and CNS tumors (5). Yet, despite promising preclinical results, clinical trials have been unimpressive and plagued by adverse events (8-10). This may reflect the poor penetration of therapeutic mAbs to brain, resulting in inadequate target engagement (11). Failures of anti-Aβ immunotherapies to engage with plaques located in deep brain structures may contribute to the lack of translation of these therapies into routine AD treatment (4, 12). Due to the invasiveness and higher degree of complications associated with injections directly to cerebrospinal fluid (CSF), therapeutic antibodies are most commonly administered by intravenous infusion (13-15). However, circulating antibodies have low penetration of the BBB, with only 0.1-0.2% entering brain (16). As a result, therapeutic antibodies are often given at doses 1000-fold greater than the concentration needed to achieve adequate binding of the target antigen in peripheral tissues (1, 17), and these high doses in clinical trials increase the prevalence of adverse events (1, 9, 18) such as amyloid-related imaging abnormalities (ARIA)(19, 20). Thus, there is a need for improved delivery of therapeutic agents to the CNS.

### SUMMARY OF INVENTION

This invention addresses the need by providing methods for improving delivery of a composition to the CNS.

In one aspect, the invention provides a method for improving delivery of a composition to a central nervous system interstitium, brain interstitium and/or a spinal cord interstitium of a subject.

The method comprises (1) enhancing glymphatic system influx and (2) delivering the composition to the central nervous system interstitium, brain interstitium and/or the spinal cord interstitium. In one embodiment, the subject can be anesthetized with a composition comprising ketamine and dexmedetomidine before the step of enhancing, the step of delivering, or both. The step of enhancing glymphatic system influx can be carried out in a number of ways. For example, the step can comprise pumping fluid through the central nervous system interstitium, or administering an agent to the subject.

The agent can be a hypertonic solution. In one example, the hypertonic solution is administered into the blood or plasma of the subject. The hypertonic solution can comprise NaCl or mannitol. In other example, the agent can include a Stat-3 inhibitor, a bone morphogenetic protein (BMP) signaling axis molecule, an antagonist of AVP (vasopressin), an antagonist of atrial natriuretic peptide (ANP), an antagonist of Angiotensin II, an antagonist of AT2R receptors, or an antagonist of AT1 receptors. In a preferred embodiment, the agent is a hypertonic saline and/or administered intravenously to the subject.

The composition can be delivered in any suitable ways, such as intracisternally or intrathecally. The composition can be delivered at about the same time or after or before the glymphatic system influx is enhanced. The composition can be an imaging composition or a therapeutic composition. The composition can comprise a small molecule, a virus, a large molecule, a peptide, an antibody, a nucleic acid (e.g., antisense molecules and RNAi agents), or a biologically active fragment thereof. In one example, the therapeutic composition comprises an antibody. The antibody can be conjugated to a ligand that facilitates transport across the blood brain barrier (a.k.a. “BBB”). For example, the ligand can specifically bind to a BBB receptor, (such as transferrin receptor, IGF-R, LDL-R, LRP1, LRP2, and LRP8).

In another aspect, the invention provides a method for treating a neurological disorder in a subject. Examples of the disorder include a neuropathy, an amyloidosis, cancer, an ocular disease or disorder, a viral or microbial infection, inflammation, ischemia, neurodegenerative disease, seizure, behavioral disorder, and lysosomal storage disease. The method comprises (1) enhancing glymphatic system influx and (2) delivering a therapeutic composition to the central nervous system interstitium, brain interstitium and/or the spinal cord interstitium. In one embodiment, the subject can be anesthetized with a composition comprising ketamine and dexmedetomidine before the step of enhancing, the step of delivering, or both. The step of enhancing glymphatic system influx can be carried out in a number of ways as mentioned above. In one example, the step comprises pumping fluid through the central nervous system interstitium. In another, the step of enhancing glymphatic system influx comprises administering an agent to the subject.

The agent can be a hypertonic solution. In one example, the hypertonic solution is administered into the blood or plasma of the subject. The hypertonic solution can comprise NaCl or mannitol. In other examples, the agent can include a Stat-3 inhibitor, a BMP signaling axis molecule, an antagonist of AVP (vasopressin), an antagonist of ANP, an antagonist of Angiotensin II, an antagonist of AT2R receptors, or an antagonist of AT1 receptors. In a preferred embodiment, the agent is a hypertonic saline and/or administered intravenously to the subject

The composition can be delivered in any suitable ways, such as intracisternally or intrathecally. The composition can be delivered at about the same time or after or before the glymphatic system influx is enhanced. The composition can comprise a small molecule, a virus, a large molecule, a peptide, an antibody, a nucleic acid (e.g., antisense molecules and RNAi agents), or a biologically active fragment thereof. In one example, the therapeutic composition comprises an antibody. The antibody can be conjugated to a ligand that facilitates transport across the blood brain barrier. For example, the ligand can specifically bind to a BBB receptor, such as transferrin receptor, IGF-R, LDL-R, LRP1, LRP2, and LRP8.

The antibody can be an anti-Aβ antibody. The subject can be a mammal, such as a human or a non-human primate. In one embodiment, the mammal is a patient in need of treatment, such as an aged or elderly person.

In yet another aspect, the invention features a kit for improving delivery of a composition (e.g., an imaging composition or a therapeutic composition) to the CNS of a subject. The kit comprises the composition and an agent that enhances glymphatic system influx. The agent can be a hypertonic solution, such as a hypertonic solution comprising NaCl or Mannitol. The agent can be a Stat-3 inhibitor, a BMP signaling axis molecule, an antagonist of AVP (vasopressin), an antagonist of ANP, an antagonist of Angiotensin II, an antagonist of AT2R receptors, or an antagonist of AT1 receptors. The composition can comprise a small molecule, a virus, a large molecule, a peptide, an antibody, a nucleic acid, or a biologically active fragment thereof. The antibody can be conjugated to a ligand that facilitates transport across the blood brain barrier. An example of the antibody is an anti-Aβ antibody.

In a further aspect, the invention provides a transcranial macroscopic imaging method. The method comprises introducing an effective amount of an imaging agent to the central nervous system of a subject, and imaging the brain of the subject. The imaging agent can be introduced intracisternally or intrathecally. In a preferred embodiment, the imaging agent comprises a fluorophore and the step of imaging comprises fluorescence macroscopy. In some examples, the fluorophore re-emit light in the infrared region (e.g., the near-infrared region, the mid-infrared region, or the far-infrared region) upon excitation.

In certain embodiments, the above-described composition, imaging composition, or therapeutic composition can contain one or more nanoparticles. The nanoparticle can include or can be linked to or conjugated to or coated with or encompassing a suitable reagent (e.g., imaging reagent, a therapeutic reagent, or both. Examples of such a reagent include a small molecule, a polymer, a virus, a large molecule, a peptide, an antibody, a nucleic acid, or a biologically active fragment thereof. The nanoparticle can be about 1 to about 500 nm in diameter (e.g., about 1 nm to about 200 nm, about 1 nm to about 200 nm, about 2 nm to about 100 nm, about 2 nm to about 100 nm, about 2 nm to about 50 nm, about 5 nm to about 20 nm, about 10 nm to about 15 nm, about 3 nm to about 10 nm, and about 3 nm to about 6 nm). In some embodiments, the polymer can be dextran, poly (amine-co-ester), poly(beta-amino-ester), polyethylenimine, poly-L-Lysine, polyethylene glycol, or dendrimers. In a preferred embodiment, the nanoparticle can be about 10 to about 15 nm in diameter.

The details of one or more embodiments of the invention are set forth in the description below. Other features, objectives, and advantages of the invention will be apparent from the description and from the claims.

### BRIEF DESCRIPTION OF THE DRAWINGS

FIGS. 1A, 1B, 1C, 1D, 1E, 1F, 1G, 1H, and 1I are a set of diagrams and photographs showing in vivo transcranial brain-wide imaging of CSF influx. FIG. 1A shows that mice were imaged through an intact skull using a macroscope. FIG. 1B shows that a fluorescent protein tracer (BSA-647 nm) was delivered into the cisterna magna (2 μL/min, 5 min) and tracer influx was imaged for 30 min from the start of the injection. All mice received i.p. isotonic saline at the onset of the intracisternal injection. FIG. 1C shows representative time-lapse images of CSF influx over the first 30 minutes following tracer injection in anesthetized (KX) and awake wild type mice, as well as anesthetized Aqp4^{-/-} mice (KX-Aqp4^{-/-}). Images (8-bit pixel depth) are color-coded to depict pixel intensity (PI) in arbitrary units (A.U.). Scale bar=2 mm. Fluorescence was detected as early as 5 min after infusion at the base of the brain approximately 5-6 mm below the dorsal cortical surface. FIG. 1D shows quantification of mean pixel intensity (MPI) for the 30-minute in vivo imaging series depicted in (c) (mean±SEM; n=5-7 mice/group; repeated measures two-way ANOVA, Sidak's multiple comparisons test; *P<0.05, **P<0.01, ***P<0.001, ****P<0.0001 vs. KX). FIG. 1E shows representative front-tracking analysis of CSF tracer influx over the imaging session. Fronts are time-coded in minutes. FIG. 1F shows quantification of the influx area over time from analysis (e) (mean±SEM; n=5-7 mice/group; repeated measures two-way ANOVA, Sidak's multiple comparisons test; ****P<0.0001 KX vs. Awake and KX-Aqp4^{-/-}). FIG. 1G shows average influx speed maps (μm/min) generated from group data in (c) and (e). FIG. 1H shows representative conventional fluorescence images of brains ex vivo upon removal from the cranium (bottom left; scale bar=2 mm) and after coronal sectioning to evaluate tracer penetrance deep into the cortical surface (top; scale bar=1 mm) in the KX and awake wild type, and KX-anesthetized Aqp4^{-/-} groups. High magnification images of perivascular tracer were acquired using laser scanning confocal microscopy (bottom right; scale bar=50 μm). FIG. 1I shows quantification of ex vivo coronal section fluorescence MPI for the KX and awake wild type, and KX-anesthetized Aqp4^{-/-} groups (mean±SEM; n=3-8 mice/group; one-way ANOVA, Tukey's multiple comparisons test; *P<0.05, **P<0.01).

FIGS. 2A, 2B, 2C, 2D, 2E, 2F, 2G. 2H, 2I, and 2J are a set of diagrams and photographs showing that plasma hypertonicity increased CSF influx in anesthetized mice. FIG. 2A shows that fluorescent BSA-647 was delivered into the cisterna magna (CM) of anesthetized mice. Mice received either isotonic saline (KX), hypertonic saline (+HTS), or hypertonic mannitol (+Mannitol) i.p. at the onset of the CM injection. FIG. 2B shows representative time-lapse images of BSA-647 influx over the immediate 30 minutes following CM injection in the KX, +HTS, and +Mannitol groups. Images (8-bit pixel depth) are color-coded to depict pixel intensity (PI) in arbitrary units (A.U.). Scale bar=2 mm. FIG. 2C shows representative front-tracking analysis of CSF tracer influx over the imaging session for all groups. Fronts are time-coded in minutes. FIG. 2D shows quantification of the influx area over time (mean±SEM; n=6-7 mice/group; repeated measures two-way ANOVA, Sidak's multiple comparisons test; ****P<0.0001 KX vs. +HTS and +Mannitol). FIG. 2E shows tracer influx speed maps (μm/min) and FIG. 2F shows quantification of mean influx speeds for all groups (mean±SEM; n=6 mice/group; one-way ANOVA, Tukey's multiple comparisons test; *P<0.05, ***P=0.001). FIG. 2G shows representative ex vivo conventional fluorescence images of intact brains upon removal from the cranium (bottom left; scale bar=2 mm) and after coronal sectioning (top; scale bar=1 mm) from all groups. Coronal sections were imaged with high-powered confocal laser scanning microscopy to evaluate perivascular tracer (bottom right; scale bar=50 μm). FIG. 2H shows quantification of ex vivo coronal section fluorescence MPI (mean±SEM; n=5-7 mice/group; one-way ANOVA, Tukey's multiple comparisons test; **P<0.01, ***P=0.003). FIG. 2I and FIG. 2H show total brain uptake of CSF-delivered (FIG. 2I) ³H-dextran (40 kDa) or (FIG. 2J) ¹⁴C-inulin (6 kDa) in all three groups (mean±SEM; n=5 mice/group; one-way ANOVA, Tukey's multiple comparisons test; **P=0.001, ***P=0.0009, ****P<0.0001). Expressed as percent injected dose (%ID). KX group same as in FIG. 1.

FIGS. 3A, 3B, 3C, 3D, 3E, 3F, 3G, and 3H are a set of diagrams and photographs showing that plasma hypertonicity overrode arousal state inhibition of glymphatic function. FIG. 3A shows head-plated, awake mice received intracisternal BSA-647. Mice received either isotonic saline (Awake), hypertonic saline (+HTS), or hypertonic mannitol (+Mannitol) i.p. at the onset of the cisterna magna (CM) injection. FIG. 3B shows representative time-lapse images of BSA-647 influx over the immediate 30 minutes following CM injection in the Awake, +HTS, and +Mannitol groups. Images (8-bit pixel depth) are color-coded to depict pixel intensity (PI) in arbitrary units (A.U.). Scale bar=2 mm. Fluorescence was first detected at the base of the brain approximately 5-6 mm below the dorsal cortical surface. FIG. 3C shows representative front-tracking analysis of CSF tracer influx over the imaging session for all groups. Fronts are time-coded in minutes. FIG. 3D shows quantification of the influx area over time (mean±SEM; n=5-7 mice/group; repeated measures two-way ANOVA, Sidak's multiple comparisons test; ****P<0.0001 Awake vs. +HTS and +Mannitol). FIG. 3E shows tracer influx speed maps (μm/min) and FIG. 3F shows quantification of mean influx speeds for all groups (mean±SEM; n=5-7 mice/group; one-way ANOVA, Tukey's multiple comparisons test; **P=0.0024, ***P=0.0003). FIG. 3G shows representative ex vivo coronal sections from all groups (scale bar=1 mm). FIG. 3H shows quantification of ex vivo coronal section fluorescence MPI (mean±SEM; n=5-6 mice/group; one-way ANOVA, Tukey's multiple comparisons test; **P=0.0063, ***P=0.003). Awake group same as in FIG. 1.

FIGS. 4A, 4B, 4C, 4D, 4E, 4F, 4G, 4H, 4I, 4J, and 4K are a set of diagrams and photographs showing that plasma hypertonicity improved the delivery of an Aβ antibody in 6-month-old APP/PS1 mice and enhanced target engagement. FIG. 4A and FIG. 4B show that plaques were labeled 24 h before with methoxy-X04 (MeX04). Mice were then anesthetized and a fluorescent anti-Aβ antibody was injected intracisternally. Mice received either i.p. isotonic saline (Control) or hypertonic saline (+HTS) at the onset of the intracisternal infusion. After 120 min, mice were perfusion-fixed with a fluorescent lectin to label the vasculature. FIG. 4C shows representative ex vivo images of intact brains upon removal from the cranium (bottom left; scale bar=2 mm) and after coronal sectioning to evaluate antibody penetrance into the brain (top; scale bar=500 μm). Confocal images of the antibody and Aβ plaques (arrows) surrounding the perivascular spaces of penetrating arteries (bottom right; scale bar=100 μm). FIG. 4D shows quantification of ex vivo coronal section Aβ antibody fluorescence MPI (mean±SEM; n=5 mice/group; unpaired two-tailed t-test; **P=0.0039). FIG. 4E shows representative high-magnification confocal images of perivascular Aβ plaques (scale bar=20 μm). FIG. 4F shows percent of target engagement shown by co-labeling of the antibody with MeX04⁺ Aβ plaques (mean±SEM; n=5 mice/group; unpaired t-test; **P=0.005). FIG. 4G shows nearest neighbor analysis of the average distance of a co-labeled plaque from its nearest perivascular space (PVS) in p.m (mean±SEM; total number of co-labeled plaques/number of mice in group; unpaired t-test; ****P<0.0001). FIG. 4H shows histogram and cumulative frequency plot of the number of co-labeled plaques and distance from the nearest PVS. FIG. 4I shows representative high-magnification confocal image with orthogonal views showing the anti-Aβ antibody engaging the surface of a plaque (arrows). Scale bar=20 μm. FIG. 4J shows three-dimensional reconstruction of Aβ plaques from an +HTS-treated mouse showing antibody targeting and engaging plaque surface (scale bar in both=20 μm). FIG. 4K shows plaque burden was the same between groups (mean±SEM; n=5 mice/group; unpaired t-test; P=0.6165).

FIGS. 5A and 5B are a set of diagrams and photographs showing a transcranial macroscopic imaging system. FIG. 5A shows optical schematic. The system uses a tunable LED illumination system that allows individual control of up to 16 different wavelengths. Excitation for 647 nm fluorophores was achieved using a 635 nm wavelength. The imaging software controls rapid switching between wavelengths and when paired with a quad filter cube enables high-speed 4 channel imaging (˜100Hz) without having to rotate the filter turret. The macroscope has a total magnification of 4-40× and with a 0.63× objective permits a long working distance (W.D.) and a high numerical aperture (N.A.) with a field of view (F.O.V.) of about 11.7 mm at the magnification used for this study. This set-up uses a scientific CMOS camera that has an effective area of 13.312×13.312 mm and a full resolution of 2048×2048 pixels, enabling fast image acquisition (100 frames per second). The system is compatible with image splitting optics for simultaneous two-channel applications for dual CSF tracer studies. FIG. 5B shows photograph of the macroscopic imaging system.

FIGS. 6A, 6B, 6C, 6D, 6E, 6F and 6G are a set of diagrams and photographs showing imaging penetration depth analysis. FIG. 6A shows that bovine serum albumin-conjugated to AlexaFluor 647 (BSA) was serially diluted from 10 to 1×10⁻⁴ mg/mL in artificial CSF (aCSF). A 10 μl drop was aliquoted into a 96-well plate and imaged on the macroscope at a 635 nm wavelength using the same magnification and exposure time used in the in vivo experiments. Images were color-coded for pixel intensity (PI) in arbitrary units (A.U.) from 0 to 255 (scale bar=2 mm). FIG. 6B shows that mean pixel intensity (MPI) was calculated for each 10 μl droplet and plotted as a function of tracer concentration. The data was fit with a variable slope sigmoidal function. The optimal dilution of tracer that is within the range of the in vivo experiments was 0.1 mg/ml. FIG. 6C shows schematic of the experimental set-up showing acute coronal sections of increasing thickness placed over a capillary filled with BSA-647 embedded in agar. FIG. 6D shows representative PI color-coded images of the fluorescent capillary in the plane of focus with: no tissue (0 μm), a 500 μm-thick, and 1,000 μm-thick coronal section placed above the capillary (scale bar=2 mm). FIG. 6E shows raw PI from 6000 μm line scans centered over the capillary acquired through coronal sections of increasing thickness between 200-4000 μm. FIG. 6F shows Gaussian fit of the raw data from (E) showed good agreement for all (R²>0.924) except the 4,000 μm (R²=0.55). FIG. 6G shows regions of interest were drawn over the capillary within the perimeter of the coronal section and MPI was measured and plotted as a function of depth. Data was fit using a one phase exponential decay function (R²=0.96) showing that fluorescent signal plateaus between 1-2 mm of tissue thickness, in agreement with results from (F).

FIGS. 7A, 7B, 7C, 7D, 7E, 7F, and 7G are a set of diagrams and photographs showing that in vivo transcranial imaging correlated with tracer transport to the dorsal cortex and ex vivo quantification at all timepoints. Anesthetized mice received cisterna magna (CM) injections of BSA-647 (BSA) during in vivo imaging and brains were extracted and fixed at 5-minute intervals between 5 and 30 minutes after the start of the infusion (n=3 mice/6 time points). FIG. 7A shows tracer transport color-coded as a function of time after CM injection in sagittal and coronal projections. FIG. 7B shows coronal sections from the 5, 15, and 30 min time point after CM injection showing that tracer was transported from the base of the brain along the lateral cortical curvature, reaching the dorsal convexity in the later time points (scale bar=2 mm). FIG. 7C shows mean pixel intensity (MPI) of the last frame from transcranial in vivo imaging correlated with the MPI of 6 coronal sections from the same brain across all 6 time points (Pearson correlation, P=0.0003). FIG. 7D shows that MPI has similar kinetics when quantified both in vivo and ex vivo and both data sets have good agreement with a one phase exponential decay function (in vivo: R²=0.838; ex vivo: R²=0.834). FIG. 7E shows that to estimate the depth of tracer, MPI in arbitrary units (A.U.) was quantified in 1 mm ROIs starting from the dorsal cortex for all time points. The presence of tracer was determined as MPI values 2 standard deviations above background (dashed line). FIG. 7F shows the estimated distance of tracer below the dorsal cortex after the start of the intracisternal injection. Error bars reflect the 1 mm wide region of interest. Data was also fit with a one phase exponential decay (R²=0.976). FIG. 7G shows in vivo, ex vivo and tracer depth from (D) and (F), respectively, were z-score transformed and fit with a one phase exponential decay function. Extra sum-of-squares F test concluded that all three datasets were fit by a single global model and did not differ significantly (P=0.288; R²=0.913) suggesting that the increase in MPI on transcranial optical imaging is correlated with the tracer moving from the base of the brain towards the dorsal cortex. Data demonstrates that fluorescence is detected as early as 5 min after CM injection, when the bulk of tracer is located 5-6 mm below the cortical surface, providing great sensitivity for whole-brain tracer quantification comparable to terminal ex vivo methods

FIGS. 8A, 8B. 8C, 8D, 8E, 8F, and 8G are a set of diagrams and photographs showing that CSF tracer influx seen in transcranial imaging occurred along perivascular spaces surrounding arteries and had similar kinetics to previous in vivo imaging modalities. FIG. 8A shows macroscopic image from a typical experiment showing brain-wide CSF tracer (BSA-647) influx along the distribution of both middle cerebral arteries (MCA) after intracisternal injection (scale bar=2 mm). FIG. 8B shows that to evaluate the anatomical pathway along which CSF inflow occurs, a separate group of mice were imaged through a cranial window using two-photon laser scanning microscopy after intracisternal injection of BSA-Texas Red (TxRd) and i.v. FITC dextran to label vasculature (scale bar=50 μm). The dura mater was left intact and the cranial window was sealed with agarose and a cover slip to prevent intracranial pressure loss. Imaging showed tracer flowing along two perivascular spaces (PVS) on each side of the MCA, below blood vessels of the dura (arrows). FIG. 8C shows a magnified image from inset in FIG. 8A and shows CSF tracer on each side of the left posterior branch of the MCA (scale bar=500 μm). FIG. 8D shows a line scan from the black line in FIG. 8C and shows high pixel intensity (PI) in arbitrary units (A.U.) on both sides of the MCA with a decrease in fluorescence over the artery. The width of the space measured in FIG. 8D is comparable to that seen in FIG. 8B. FIG. 8E shows orthogonal reconstructions from the blue line in FIG. 8B and shows that CSF tracer is confined to the subpial PVS around the MCA and not within the subarachnoid space (SAS; scale bar=50 μm). FIG. 8F shows quantification from the mean of 3 perivascular regions of interest along the MCA normalized to the maximum fluorescence intensity (ΔF/Fₘₐₓ) of the imaging session expressed as a percent. Higher baseline background fluorescence is seen in 2-P due to bleed through from the vascular label channel. FIG. 8G shows time to tracer appearance after the start of the intracisternal injections. (mean±SEM; n=4-5 mice/group; ns: not significant; unpaired t-test; P=0.8761).

FIG. 9 is a set of photographs showing transcranial macroscopic imaging of CSF influx pathways. After tracer delivery into the cisterna magna it is possible to identify several intracranial structures through the intact skull (dashed line). (Top panel) In anesthetized mice, meningeal structures such as the olfactory sinus, superior sagittal sinus, and the left and right transverse sinuses can be observed (blue). As previously shown, tracer is first found in the large pools of subarachnoid CSF surrounding the brain like those around the olfactofrontal cistern (purple) and the pineal recess (green). Brain uptake of the tracer occurs within the perivascular spaces of pial arteries (red), particularly following the distribution of the anterior and dorsal cortical segments of the middle cerebral artery, and then continues down into the brain along penetrating arteries. Eventually the tracer can be found in the perivenous spaces of the cortical bridging veins and surrounding the meningeal sinuses. (Bottom panel) In awake mice, some of the most anterior and posterior structures are covered by the headplate but all pial perivascular spaces can be readily identified. This approach can also be used for chronic imaging as it is still possible to identify tracer fluxes through transparent dental cement as can be seen on the edges of the headplate.

FIG. 10 is a set of photographs showing that CSF tracer inflow routes imaged through the intact skull were also found in the ex vivo brain. (Center) Ex vivo whole brain imaging from an anesthetized mouse, 30 minutes after intracisternal injection (scale bar=2 mm). (Left) Higher magnification insets from the left cortical surface (dark blue) showing that CSF tracers can be found along branches of the anterior middle cerebral artery (aMCA) and posterior MCA (pMCA), traced in red (scale bar=1 mm). (Right) Insets from the right cortex (light blue) demonstrating that tracer influx occurs along the same segments of the aMCA and pMCA (red traces; scale bar=1 mm).

FIGS. 11A, 11B, 11C, 11D, and 11E are a table and a set of diagrams showing inducing plasma hyperosmolarity. FIG. 11A shows solutions composition and dose used throughout the study. FIG. 11B shows measured osmolality of plasma tonicity-shifting solutions. FIG. 11C shows plasma osmolality at 30 minutes after intraperitoneal injection in the control, +HTS, and +Mannitol groups for both the anesthetized (KX) and awake conditions (mean±SEM; n=5-15 mice/group; ordinary two-way ANOVA, Tukey's multiple comparisons test; *P=0.0151, ***P=0.0001, ****P<0.0001). FIG. 11D shows plasma Na⁺ ([Na⁺]_{Plasma}) and FIG. 11E shows Cl⁻ ([Cl⁻]_{Plasma}) concentration 30 min after i.p. injection. High [Na⁺]_{Plasma} and [Cl⁻]_{Plasma} in the +HTS groups account for the hyperosmolarity seen in (c). Mannitol-induced plasma hyperosmolarity does not affect [Na⁺]_{Plasma} and [Cl⁻]_{Plasma} and is produced by an elevated osmolal gap. (mean±SEM; n=5 mice/group; ordinary two-way ANOVA, Tukey's multiple comparisons test; ***P<0.001, ****P<0.0001).

FIGS. 12A, 12B, and 12C are a set of diagrams and photographs showing that Manipulations of plasma tonicity did not disrupt the blood-brain barrier. FIG. 12A shows representative ex vivo coronal section images of FITC-dextran (1% m/v, 70 kDa) extravasation 30 minutes following intraperitoneal isotonic saline (KX), hypertonic saline (+HTS), and hypertonic mannitol (+Mannitol) solution administration in anesthetized animals. Positive controls received intracarotid 2M mannitol (+IC Mannitol). (scale bar=1 mm). FIG. 12B shows quantification of thresholded fluorescence expressed as percent area from 6 coronal sections depicted in FIG. 12A revealed no significant increases in extravasated FITC-dextran between the experimental groups but did show a significant difference between the experimental groups and the positive control, (mean±SEM; n=5-6 mice/group; one-way ANOVA, Tukey's multiple comparisons test, ns: not significant, P>0.999; ***P<0 .0002). FIG. 12C shows plasma concentration of the FITC-dextran was evaluated spectrophotometrically and revealed no significant differences between any of the experimental groups but did show increased extravasation of the dextran in the positive control group (mean±SEM; n=5-6 mice/group; one-way ANOVA, Tukey's multiple comparisons test, *P=0.0426).

FIGS. 13A and 13B are a set of photographs and a diagram showing that plasma hypertonicity overrode glymphatic inhibition in Aqp4^{-/-} mice. Fluorescent BSA-647 was delivered into the cisterna magna (CM) of anesthetized Aqp4^{-/-} mice. Mice received either hypertonic saline (Aqp4^{-/-}+HTS), or hypertonic mannitol (Aqp4^{-/-}+Mannitol) i.p. at the onset of the CM injection. FIG. 13A shows representative time-lapse images of BSA-647 influx over the immediate 30 minutes following CM injection in the Aqp4^{-/-}+HTS, and Aqp4^{-/-}+Mannitol groups. Images (8-bit pixel depth, 0-255) are color-coded to depict pixel intensity (PI) in arbitrary units (A.U.). Scale bar=2 mm. FIG. 13B shows quantification of the mean pixel intensity (MPI) over time compared to the wild type groups from FIG. 2 (WT+HTS, WT+Mannitol; mean±SEM; n=3-5 mice/group; repeated measures two-way ANOVA, Sidak's multiple comparisons test; group effect: P=0.1029; ns: not significant).

FIGS. 14A, 14B, 14C, and 14D are a set of diagrams and photographs showing that in vivo transcranial imaging correlated with ex vivo quantification of fluorescent and radio-labeled tracers. FIG. 14A shows that images acquired at 30 min after in vivo imaging were analyzed for mean pixel intensity (MPI; purple) and influx area using front-tracking software (green). Mice were fixed and images of the dorsal whole brain (blue) and coronal sections (red) were acquired from the same brain using the macroscope. FIG. 14B shows that Z-scores were calculated for all outcomes and a multiple linear regression model was generated from the data and plotted with 95% confidence intervals. All metrics had a significant positive linear relationship and were strongly correlated with ex vivo coronal sections (Whole Brain: R²=0.8473; In vivo MPI: R²=0.7354; In vivo Influx Area: R²=0.8355). The slopes of all three regressions were not significantly different from each other (P=0.8209). FIG. 14C and FIG. 14D show that fluorescent tracer quantification from (FIG. 14C) ex vivo coronal sections and (FIG. 14D) in vivo imaging (influx area) was also tightly correlated with quantification of two separate radiotracers: ³H-dextran (40 kDa) and ¹⁴C-inulin (6 kDa) using plasma osmolality as the predictor (Coronal sections: R²=0.589; In vivo: R²=0.8072; ³H-Dextran: R²=0.6832; ¹⁴C-Inulin: R²=0.6973). The overall slopes of all regressions were not significantly different (P>0.05).

FIGS. 15A, 15B, 15C, and 15D are a set of diagrams showing that plasma hyperosmolarity caused a decrease in intracranial pressure and interstitial fluid volume without altering mean arterial blood pressure or cerebral blood flow. FIG. 15A shows that mean arterial blood pressure (MAP) in the femoral artery of anesthetized mice (KX) was recorded in mmHg, starting 5 min before i.p. injection of isotonic saline (Control), hypertonic saline (+HTS), and hypertonic mannitol solution (+Mannitol), for 30 minutes (mean±SEM n=4-5 mice/group; repeated measures two-way ANOVA, Tukey's multiple comparisons test; **P<0.01, color-coded asterisks denote a difference between KX and +HTS or KX and +Mannitol at different time points). FIG. 15B shows that relative cerebral blood flow (rCBF; pressure units, p.U.) was measured using laser Doppler flowmetry (mean±SEM; n=3-5 mice/group; repeated measures two-way ANOVA, Tukey's multiple comparisons test; **P<0.01, color-coded asterisks denote a difference between KX and +Mannitol at different time points). FIG. 15C shows intracranial pressure (ICP) recording for the 5 minutes prior to and 30 minutes following i.p. injection at 0 minutes (mean±SEM; n=4-5 mice/group; repeated measures two-way ANOVA, Tukey's multiple comparisons test; ****P<0.0001). FIG. 15D shows brain water content at 30 minutes following i.p. injection in the control and hypertonic groups (mean±SEM; n=4-10 mice/group; ordinary two-way ANOVA, Tukey's multiple comparisons test; ***P=0.0001, ****P<0.0001).

FIG. 16 is a set of diagrams showing a three-compartment model of the relationship between blood plasma, brain, and CSF under isotonic and hypertonic conditions. In the situation of an isotonic blood plasma, there is no change in interstitial fluid volume (V_{ISF}; brain water content, BWC) or pressure (P_{ISF}; intracranial pressure, ICP), and as a result there is no change in the net direction or magnitude of glymphatic flow. In the hypertonic condition, with increased plasma osmolyte content there will be a net resorption of ISF, resulting in decreased ISF volume, and a negative ISF pressure that will enhance CSF influx into brain.

FIG. 17 is a set of diagrams and photographs showing transcranial optical imaging and Aβ antibody delivery into CSF (left panel) and CSF tracers and Aβ antibody under isotonic and hypertonic conditions (right panel).

FIGS. 18A, 18B, 18C, 18D, and 18E are diagrams showing preparation of small gold nanoparticles to be visualized with either single-photon emission tomography or magnetic resonance imaging. FIG. 18A shows schematic illustration of a PEG coated AuNP, labelled with ¹¹¹In-LA-DOTA (bottom left) or Gd-LA-DOTA (bottom right). FIG. 18B shows background: Transmission electron microscopy image of PEG₂₀₀₀ coated AuNPs. Insert: High resolution image of a single AuNP. Front: Size distribution of PEG₂₀₀₀ coated AuNPs measured by transmission electron microscopy. FIG. 18C shows example UV-VIS spectra of citrate-stabilized (brown) and PEG₂₀₀₀ coated (blue) AuNPs. FIG. 18D shows SEC separation of selected sample mixtures. ¹¹¹In—AuNPs: Absorption at 515 nm (dark grey, right axis) and radioactivity (light grey, left axis). Free ¹¹¹In-LA-DOTA complex: Radioactivity (light green, left axis). ¹¹¹In-LA-DOTA mixed with brain homogenate after one hour: Radioactivity (dark green, left axis). FIG. 18E shows stability of ¹¹¹In-LA-DOTA labelled AuNPs in rat BH (grey) or rat CSF (blue) at 37° C., depicted as percentage of the total radioactivity associated with the AuNP fraction after SEC separation. The t=0 data was obtained immediately before mixing with the tissue extracts. Error shown as standard deviation (n=3). abs., absorption; AuNP(s), gold nanoparticle(s); BH, brain homogenate; CSF, cerebrospinal fluid; ¹¹¹In—AuNPs, ¹¹¹Indium-labelled gold nanoparticles; ¹¹¹In-LA-DOTA, ¹¹¹Indium-labelled linker (lipoic acid-1,4,7,10-tetraazacyclododecane-1,4,7,10-tetraacetic acid); SPECT, single-photon emission tomography; MRI, magnetic resonance imaging; PEG, polyethylene glycol; rad., radioactivity; SEC, size exclusion chromatography.

FIGS. 19A, 19B, 19C, 19D, 19E, 19F, 19G, 19H, 19I, 19J, and 19K are diagrams and photographs showing hypertonic saline treatment enhances the delivery of intrathecally infused small gold nanoparticles to the brain. FIG. 19A shows experimental setup used for SPECT imaging of the transport of ¹¹¹In—AuNP or ¹¹¹In-LA-DOTA infused to the cisterna magna of rats under ketamine/dexmedetomidine anesthesia. FIG. 19B shows experimental timeline. Rats received an intraperitoneal injection of either isotonic (VEH, n=8) or hypertonic saline (HTS, n=6). ¹¹¹In—AuNP dispersion was infused to the cisterna magna and 180 minutes of SPECT was acquired interspersed with CT. A third group was given an isotonic intraperitoneal injection and an intracisternal infusion of ¹¹¹In-LA-DOTA (VEH LA-DOTA, n=5). FIG. 19C shows three-dimensional rendering of ¹¹¹In—AuNP SPECT overlaid on a CT image of a representative rat in the VEH group shows the distribution of ¹¹¹In—AuNP along the glymphatic pathway. FIG. 19D shows three-dimensional rendering of population-based average SPECT images after infusion of ¹¹¹In—AuNP overlaid on a T2-weighted MRI brain template. FIG. 19E shows intracranial exposure to ¹¹¹In—AuNPs increased by 50% and residual ¹¹¹In—AuNP content at 3 hours doubled in the HTS group compared with VEH as demonstrated by the time-activity curve (left) and AUC₀₋₃ₕ (right) of ¹¹¹In—AuNP. FIG. 19F and FIG. 19G show comparison of time-activity-curves (left) and AUCs (right) of ¹¹¹In—AuNP or ¹¹¹In-LA-DOTA after segmenting the intracranial compartment into brain (FIG. 19F) and CSF (FIG. 19G). FIG. 19H shows that striatal regions of interest were deep in brain tissue to avoid spill-over activity from the subarachnoid space. Coronal slices of group-wise average ¹¹¹In—AuNP images show the distribution at 180 minutes after infusion. FIG. 19I shows that analysis of time-activity-curves from striatal regions of interest show 3-fold increases in exposure and peak concentration of ¹¹¹In—AuNPs. FIG. 19J shows that a region of interest was placed in the bridge between thalamic hemispheres to maximize distance from the subarachnoid space. Group-wise population-based average ¹¹¹In—AuNP distribution in a sagittal slice 180 minutes after infusion. FIG. 19K shows that analysis of time-activity-curves from thalamic regions of interest show 10-fold increase in exposure and 5-fold increase in peak concentration of ¹¹¹In—AuNPs by HTS treatment. AUC₀₋₃, area under the time-activity-curve from 0 to 3 hours; CT, computed tomography; HTS, hypertonic saline; ¹¹¹In—AuNPs, ¹¹¹indium-labelled gold nanoparticles; ¹¹¹In-LA-DOTA, ¹¹¹indium-labelled linker (lipoic acid-1,4,7,10-tetraazacyclododecane-1,4,7,10-tetraacetic acid); MRI, magnetic resonance imaging; SPECT, single-photon emission tomography; VEH: vehicle/control group; %ID: percent of the infused dose. *: p<0.05, **: p<0.01, ***: p<0.001, ****: p<0.0001. Quantitation and statistics are detailed in Table 3.

FIGS. 20A, 20B, 20C, 20D, 20E, and 20F are diagrams and photographs showing brain delivery of gadolinium-labelled small gold nanoparticles in the perivascular spaces of pial and penetrating arteries demonstrated with dynamic contrast-enhanced magnetic resonance imaging. FIG. 20A shows experimental setup used for DCE-MRI of the transport of Gd—AuNP infused to the cisterna magna of rats after hypertonic saline treatment. FIG. 20B shows experimental timeline. Anesthetized rats were given an intraperitoneal injection of hypertonic saline (HTS, n=5), and 5 minutes later, Gd—AuNP dispersion was infused to the cisterna magna and 180 minutes of DCE-MRI was acquired. FIG. 20C shows representative sagittal and coronal slices from one rat shows the signal increase, mostly in periarterial spaces, caused by shortening T1 relaxation-time in the presence of paramagnetic gadolinium. FIG. 20D shows representative three-dimensional rendering in false color shows the distribution of Gd—AuNPs across the perivascular network of the middle cerebral artery at 60 minutes with most of them distributed across the subarachnoid space and brain at 180 minutes. FIG. 20E shows that quantification of Gd—AuNP as percentage signal increase in the perivascular space of the middle cerebral artery shows that concentration peaks 50 minutes after the beginning of the infusion and is reduced to a third of the peak at 180 minutes. FIG. 20F shows that to increase sensitivity to the Gd—AuNP signal, inventors averaged all DCE-MRI frames from before (baseline) and after infusion (avg. 0-180 minutes) and created a maximum-intensity projection of the region delineated (left). Post-infusion signal enhancement is clearly seen in deep striatal perivascular spaces (green arrows). Gd—AuNP, gadolinium-labelled gold nanoparticle; DCE-MRI, dynamic contrast-enhanced magnetic resonance imaging; HTS, hypertonic saline; Inf., infusion; MCA, middle cerebral artery; PVS, perivascular space; ref., reference.

FIGS. 21A, 21B, 21C, 21D, 21E, and 21F are diagrams showing decreased egress of small gold nanoparticles to the lymphatic structures of the head and neck after hypertonic saline treatment. FIG. 21A shows three-dimensional rendering of vehicle-group average (VEH, n=8) ¹¹¹In—AuNP SPECT overlaid on a CT template shows visible efflux routes from the intracranial compartment. Efflux routes include caudal-directed flow in the subarachnoid space of the spinal canal and the rostral egress through the cribriform plate and nasal turbinates towards the deep cervical lymph nodes. ROIs for quantification of ¹¹¹In—AuNP exposure are illustrated with dashed lines. FIG. 21B shows that comparison between average SPECT from VEH (n=8) and HTS (n=6) groups shows that efflux through nasal route and to deep lymph nodes appears reduced or delayed with HTS treatment. FIG. 21C shows that no significant difference in ¹¹¹In—AuNP exposure (AUC) between HTS and VEH groups was seen in the cervical spine. FIG. 21C, FIG. 21D, FIG. 21E, and FIG. 21F show that AuNP exposure was significantly reduced with HTS treatment in nasal turbinates (FIG. 21D), pharyngeal lymph structures (FIG. 21E), and deep lymph nodes (FIG. 21F). In contrast, the availability of the small-molecular ¹¹¹In-LA-DOTA linker was significantly reduced in nasal turbinates (FIG. 21D), pharyngeal lymph structures (FIG. 21E), and virtually abolished in deep lymph nodes (FIG. 21F). AUC₀₋₃ₕ, area under the time-activity-curve from 0 to 3 hours; CT, computed tomography; HTS, hypertonic saline; Inf., infusion; ¹¹¹In—AuNP ¹¹¹ⁱndium-labelled gold nanoparticle; VEH, vehicle/control group, ***: p<0.001, ****: p<0.0001. Quantitation and statistics are detailed in Table 4.

FIGS. 22A, 22B, 22C, 22D, 22E, 22F, 22G, 22H, 22I, 22J, 22K, 22L, 22M, and 22N are diagrams showing whole-body elimination of intrathecally administered ¹¹¹In-labelled gold nanoparticles. FIG. 22A shows experimental timeline. Rats were anesthetized with ketamine/dexmedetomidine and given an intraperitoneal injection of either isotonic (VEH, n=6) or hypertonic saline (HTS, n=5). After infusion of ¹¹¹In—AuNP dispersion to the cisterna magna, rats were returned to their home cage, and full-body SPECT and CT were acquired at 1.5, 3, 4.5, 6 and 24 hours after infusion. Between 3 and 4.5 hours, the rats recovered from initial anesthesia, and they were briefly re-anaesthetized with isoflurane (1.5%) for subsequent scans. FIG. 22B shows whole-body population-based average SPECT images after infusion of ¹¹¹In—AuNP overlaid on a full body CT template. FIG. 22C shows illustration of regions of interest. FIG. 22D shows three-dimensional renderings of population-based average SPECT images after infusion of ¹¹¹In—AuNP overlaid on a T2-weighted MRI brain template. FIG. 22E to FIG. 22L show comparisons of time-activity-curves of ¹¹¹In—AuNP in regions of interest. FIG. 22M shows residual ¹¹¹In—AuNP mass or concentration at 24 hours in organs of interest. FIG. 22N shows residual ¹¹¹In—AuNP distribution at 24 hours depicted with a maximum intensity projection overlaid on an MRI template. The 24-hour time point has been shown with tenfold increased contrast compared with FIG. 22D. AUC₀₋₂₄: area under the time-activity-curve between 0 to 24 hours; HTS, hypertonic saline; ¹¹¹In—AuNP, ¹¹¹Indium-labelled gold nanoparticle; VEH: vehicle/control group *: p<0.05, **: p<0.01, ****: p<0.0001. Quantitation and statistics are detailed in Table 5.

FIG. 23 is a radio-TLC chromatogram of the ¹¹¹In-LA-DOTA complex. Eluent: methanol:water 50:50 with 5% w/v ammonium acetate, normal phase conditions on silica plates. The first peak corresponds to 18.5% of the total activity.

FIG. 24 is a radio-TLC chromatogram of the ¹¹¹In-DOTA complex. Eluent: methanol:water 50:50 with 5% w/v ammonium acetate, normal phase conditions on silica plates. Rf=0.29.

FIG. 25 is a radio-TLC chromatogram showing the complex ¹¹¹In-LA-DOTA at t=0, immediately after mixing with CSF. Eluent: methanol:water 50:50 with 5% ammonium acetate, normal phase conditions on silica plates.

FIG. 26 is a radio-TLC chromatogram showing after 4 hours incubation at 37° C. with CSF. Eluent: methanol:water 50:50 with 5% ammonium acetate, normal phase conditions on silica plates.

FIG. 27 is a radio-TLC chromatogram showing the complex ¹¹¹In-LA-DOTA after 24 hours incubation at 37° C. with CSF. Eluent: methanol:water 50:50 with 5% ammonium acetate, normal phase conditions on silica plates.

FIG. 28 is a radio-TLC chromatogram showing the complex ¹¹¹In-LA-DOTA after 48 hours incubation at 37° C. with CSF. Eluent: methanol:water 50:50 with 5% ammonium acetate, normal phase conditions on silica plates.

FIGS. 29A, 29B, 29C, 29D, 29E, 29F, 29G, 29H, and 29I are a set of filtration chromatograms for ¹¹¹In—AuNPs incubated at 37° C. in cerebrospinal fluid. The activity of each fraction given as % of the most active fraction is displayed in black (left axis) and the absorbance at 515 nm (corresponding to the NPs maximal absorption) in red (right axis). Graphs for sample CSF1: after 4 hours (FIG. 29A), after 24 hours (FIG. 29B) and after 48 hours (FIG. 29C). Graphs for sample CSF2: after 4 hours (FIG. 29D), after 24 hours (FIG. 29E) and after 48 hours (FIG. 29F). Graphs for sample CSF3: after 4 hours (FIG. 29G), after 24 hours (FIG. 29H) and after 48 hours (FIG. 29I). The disassociation of the radiolabel over the 48 h is observed as a separation of the signal for absorbance (the AuNPs) and the radioactivity (the radiolabel).

FIGS. 30A, 30B, 30C, 30D, 30E, 30F, 30G, 30H, and 30I are a set of filtration chromatograms for nanoparticles incubated at 37° C. in brain homogenate. The activity of each fraction given as % of the most active fraction is displayed in black (left axis) and the absorbance at 515 nm (corresponding to the NPs maximal absorption) in red (right axis). Graphs for sample Brain1: after 4 hours (FIG. 30A), after 24 hours (FIG. 30B) and after 48 hours (FIG. 30C). Graphs for sample Brain2: after 4 hours (FIG. 30D), after 24 hours (FIG. 30E) and after 48 hours (FIG. 30F). Graphs for sample Brain3: after 4 hours (FIG. 30G), after 24 hours (FIG. 30H) and after 48 hours (FIG. 30I). The disassociation of the radiolabel over the 48 h is observed as a separation of the signal for absorbance (the AuNPs) and the radioactivity (the radiolabel).

### DETAILED DESCRIPTION OF THE INVENTION

Despite the initial promise of immunotherapy for CNS disease, multiple recent clinical trials have failed. This may be due in part to characteristically low penetration of antibodies to cerebrospinal fluid (CSF) and brain parenchyma, resulting in poor target engagement. There is a need for improved delivery of therapeutic antibodies as well as other agents to the CNS.

This invention discloses utilizing novel transcranial macroscopic imaging to non-invasively evaluate in vivo delivery pathways of CSF fluorescent tracers. Tracers in CSF proved to be distributed through a brain-wide network of periarterial spaces, denoted as the glymphatic system. Unexpectedly, it was found that CSF tracer entry could be enhanced substantially by increasing plasma osmolality without disruption of the blood-brain barrier. Further, it was unexpected that plasma hyperosmolality overrode the inhibition of glymphatic transport that characterizes the awake state and reversed glymphatic suppression in a mouse model of Alzheimer's disease. As disclosed herein, plasma hyperosmolality enhanced the delivery of an amyloid-β (Aβ) antibody, obtaining a 5-fold increase in antibody binding to Aβ plaques. Thus, manipulation of glymphatic activity represents a novel strategy for improving penetration of therapeutic agents such as antibodies to the CNS.

A bulk flow pathway exists along the perivascular space (PVS) surrounding the pial and penetrating arteries for CSF circulation into the brain (21-25). Although antibodies show limited diffusive transport in the CNS extracellular space (26, 27), harnessing perivascular and parenchymal convective flows can enhance their delivery into the brain. This bulk flow pathway, termed the glymphatic system for its role in solute clearance and its dependence on the glial water channel aquaporin-4 (AQP4) (21), represents an ideal mechanism for drug delivery to the CNS. The fast, convective fluid flow within the glymphatic system effectively delivers solutes of high molecular weight (21) but is strongly regulated by brain state (28), aging (29), arterial pulsatility (30), and body posture (31).

This invention provides a novel non-invasive transcranial macroscopic imaging approach that allows one to track cortical CSF flow in real time in the intact brain of living mice. In this invention, this technique was used to evaluate if therapeutic enhancement of glymphatic influx would increase the delivery of CSF-based tracers into the brain. It was unexpectedly found that increasing plasma osmolality with a hypertonic solution, such as hypertonic saline or mannitol, increased glymphatic influx without disruption of the BBB.

Disclosed herein is the first study describing the use of non-invasive transcranial macroscopic imaging to evaluate CSF flow patterns in rodents. As disclosed in the working examples below, inventors observed advective tracer inflow within the leptomeningeal PVS surrounding large cerebral arteries, which matched findings in previously validated radiometric and fluorescent ex vivo quantification methods (21). Moreover, macroscopic imaging corroborated prior findings in relation to arousal state and AQP4 expression (21). Importantly, it was demonstrated that hyperosmolar therapy with e.g., intraperitoneal hypertonic saline or mannitol, doubled the penetration of an intracisternally-delivered CSF tracer, while increasing influx speeds by about 70%. This response is attributed to an increase in ISF-to-plasma efflux, causing a decrease in ICP without BBB disruption (FIG. 16). Although controlled opening of the BBB, allowing greater entry of drugs from blood to the CNS, has shown promissory results in improving drug delivery; the effect of this intervention on brain function and glymphatic clearance are yet unknown, requiring further evaluation. This intervention overcame the suppression of CSF inflow that characterizes the awake state, AQP4 depletion, and the diseased AD brain (28, 35). More specifically, plasma hypertonicity sharply improved delivery of fluorophore-conjugated Aβ antibody. Brain-wide distribution of the antibody resulted in significantly higher plaque engagement, with targeted plaques lying distinctly farther from the PVS despite a short CSF circulation time. Hyperosmolar therapy with intravenous hypertonic solutions is already clinically approved for the treatment of cerebral edema (49). Hyperosmolar therapy should enhance immunotherapy delivery deep within the brain parenchyma. Although antibodies are large molecules, proteins as large as 2,000 kDa can enter the brain parenchyma after intracisternal delivery (21, 26). Indeed, under pathological conditions such as AD, antibodies (>100 kDa) are transported through the PVS (27). Since this transport is primarily mediated by bulk flow, transport of smaller molecules can also be likewise enhanced.

The study disclosed herein shows that plasma hypertonicity can rescue impaired glymphatic function in a murine AD model, enhancing the delivery and target engagement of passive immunotherapeutics against Aβ. The study also show that one can use substantially less antibody than required in previous studies, while achieving greater target engagement (27, 58). As disclosed herein, exploiting hyperosmotic treatment to overcome the declining glymphatic flux in the awake state, in aging, and in disease can be combined with convection enhanced delivery strategies.

### Plasma Hyperosmolality and Increased CSF Influx

The present invention provides a method for improving delivery of a composition to a central nervous system interstitium, brain interstitium and/or a spinal cord interstitium of a subject comprising. The method includes enhancing glymphatic system influx and delivering the composition to the central nervous system interstitium, brain interstitium and/or the spinal cord interstitium.

One can enhance glymphatic system influx via a number of ways. For example, one can pump fluid through the central nervous system interstitium using methods and agents known in the art such as those described in WO2014130777. For instance, enhancing glymphatic system influx can comprise a step of administering an agent to a subject (such as a mammal) that increases glymphatic clearance, e.g., a Stat-3 inhibitor or BMP signaling axis molecules. In other embodiments, the agent is an antagonist of AVP (vasopressin) such as tolvaptan, conivaptan, or VPA-985, an antagonist of atrial natriuretic peptide (ANP) such as anantin, an antagonist of Angiotensin II such as losartan, an antagonist of AT2R receptors such as PD12331 9, or an antagonist of AT1 receptors such as valsartan. In another embodiment, the agent is an agent for use in the treatment of insomnia or as an aid for sleep, including but not limited to those listed below:

| Types of agent | Examples |
|---|---|
| Antihistamines | ALLEGRA ® (Fexofenadine), BENADRYL ® (Diphenhydramine), |
| | CLARITIN ® or TAVIST ® (loratadine), CHLOR-T RIM ETON ® |
| | (chlorpheniramine maleate), DIMETANE ® (Brompheniramine, |
| | Phenylpropanolamine), and ZYRTEC ® (Cetirizine) |
| Nonprescription | Unisom Nighttime Sleep-Aid, Dormin, Nytol, Simply Sleep, |
| sleep aids | Sominex, Extra Strength Tylenol PM, Diphenhydramine |
| | hydrochloride, and Excedrin P.M. |
| Benzodiazepines: | PROSUM ® (estazolam), DALMANE ® (flurazepam), DORAL ® |
| | (quazepam), RESTORIL ® (temazepam), HALCION ® (triazolam), |
| | and VALIUM ® (diazepam) |
| Non-benzodiazepines: | Imidazopyridines: AMBIEN ®, AMBIEN ® CR, INTERMEZZO ® |
| | (Zolpidem) (class of its own), and SONATA ® (pyrazolopyrimidine) |
| | (class of its own) |
| | Melatonin receptor stimulator: ROZEREM ® (ramelteon), NOTED ® |
| | (chloral hydrate), PRECEDEX ® (dexmedetomidine hydrochloride), |
| | and LUNESTA ® (eszopiclone) |
| Barbiturates | NEMBUTAL ® (phenobarbital), MEBARAL ® (mephobarbital), and |
| | Amytal Sodium (amobarbital sodium), BUTISOL ® (butabarbital |
| | sodium), and SECONAL ® Sodium Pulvules (secobarbital sodium) |

In another embodiment, the agent can be an agent that prevents AQP4 depolarization or loss of AQP4 polarization, such as JNJ-1 7299425 or JNJ-17306861. In another embodiment, the step of increasing glymphatic influx comprises the step of pumping fluid through the central nervous system interstitium. Pumping can be accomplished by any device or method known in the art, for example, by using a mechanical pump, an infusion pump, etc.

Alternatively, the step of enhancing glymphatic system influx comprises administering a hypertonic agent to the subject. Preferably, the hypertonic agent is a hypertonic solution, which can be administered into plasma of the subject.

Each of the agents described above can be used alone or in combination with one or more of the other agents.

As used herein, “hypertonic” and “hypotonic” are relative terms e.g., in relation to physiological osmolality, but can diverge from this so long as the ultimate goal of an osmotic differential or gradient is achieved between two compartments (such as the blood plasma and the central nervous system interstitium) so as to promote the influx of glymphatic flow into central nervous system interstitium, brain interstitium and/or a spinal cord interstitium. Accordingly, a “hypertonic solution” refers any physiologically and/or pharmaceutically acceptable solution that is hypertonic with respect to physiological osmolality, including hypertonic saline or sugar solutions. As mentioned herein, hypertonic solutions preferred in this invention does not cause BBB disruption.

The methods of the invention provide an agent (e.g., a pharmaceutical preparation) for injection that is hypertonic with respect to blood. To determine whether a pharmaceutical preparation is hypertonic with respect to blood, one calculates the osmolarity for all chemical components of a solution including the diluent. Tonicity can be calculated for fluids and dissolved or diluted medications, which are expressed in a numerical value of milliosmoles per liter of fluid (mOsm/L) or per kilogram of solvent (mOsm/kg). These two values also known as osmolarity and osmolality, respectively. The osmolarity of blood ranges between 285 and 310 mOsm/L and the osmolality of blood ranges between 275 and 299 mOsm/kg.

Solution osmolarity is based in part on the concepts of osmosis and osmotic pressure. Osmosis is the diffusion of solutes (dissolved particles) or the transfer of fluid through semipermeable membranes such as blood vessels or cell membranes. Osmotic pressure, which facilitates the transport of molecules across membranes, is expressed in osmolar concentrations and is referred to as hypo-osmotic (hypotonic), iso-osmotic (isotonic), or hyper-osmotic (hypertonic) when compared with biologic fluids such as blood or plasma. The term “tonicity” and “osmotic pressure” are often considered synonymous.

The osmotic pressure is the hydrostatic (or hydraulic) pressure required to oppose the movement of water through a semipermeable membrane in response to an ‘osmotic gradient’ (i.e., differing particle concentrations on the two sides of the membrane). Serum osmolality can be measured by use of an osmometer (see Example 3 below) or it can be calculated as the sum of the concentrations of the solutes present in the solution.

As used herein, tonicity and osmotic pressure are to be considered synonymously, and are to be understood broadly. Tonicity can mean the effective osmolality and is equal to the sum of the concentrations of the solutes in a solution that have the capacity to exert an osmotic force across a membrane, including a cell membrane. In the strict sense, osmolality is a property of a particular solution and is independent of any membrane. Tonicity is a property of a solution in reference to a particular membrane. However, the invention shall refer to solutions being isotonic, hypertonic, or hypotonic with respect to biological solutions such as blood or plasma, and this referencing shall include the meaning that the particular solution is isotonic hypertonic, or hypotonic with blood or plasma with respect to a cell membrane of a cell in the blood or plasma or other biological solution.

An operational definition of tonicity can be used to explain the term. This can be based on an experiment of adding a test solution to whole blood and observing the result. If the RBCs in whole blood swell and rupture, the test solution is said to be hypotonic compared to normal plasma. If the RBCs shrink and become crenate, the test solution is said to be hypertonic compared to normal plasma. If the RBCs stay the same, the test solution is said to be isotonic with plasma. The RBC cell membrane can be the reference membrane. For example, whole blood placed in normal saline (i.e., 0.9% sodium chloride) will not swell, and hence normal saline is said to be isotonic.

The methods described herein include administering to a subject a pharmaceutical solution or preparation that is hypertonic with respect to plasma or blood. As hypertonic solutions, once injected into blood, may cause fluid shifts out of cells and a variety of negative effects, care should be taken to select a proper osmolality that are not so hypertonic as to cause significant thrombosis and/or vessel irritation. In one embodiment, the solution/preparation is considered to have suitable osmolality if 30 minute after injection into a subject in the manner described in the working example below, the resulting plasma osmolality is greater than about 320 mOsml.kg⁻¹ and less than about 600 mOsml.kg⁻¹, e.g., greater than about 340 or 350 and less than about 375, 400, 425, 450, 475, 500, or about 575 mOsml.kg⁻¹. In general, hypertonic solutions useful in this invention exhibit a tonicity that is greater than about 320 mOsml.kg⁻¹, e.g., 340 to 3,000 (e.g., 500 to 2,000, 1,000 to 2,000, 1,500 to 1,800) mOsml.kg⁻¹. Solutions with an osmolality that is greater than about 600 mOsml.kg⁻¹ should be used with care in injections.

Various primary bulking agents can be used for preparing a hypertonic solution/preparation for intravenous injection. Examples include ionizing agents, e.g., NaCl, and non-ionizing. Examples of non-ionizing bulking agents include, but are not limited to, mannitol, glycine, sucrose, lactose, other disaccharides, therapeutic proteins or the active ingredient of a formulation itself, or other bulking agents known to one skilled in the art. The concentrations of non-ionizing bulking agents do not significantly affect whether a solution has a sufficient ionic strength. However, their concentrations do have an effect on osmolarity, and therefore, their concentrations can have an effect on tonicity. In certain examples, NaCl or mannitol is used. The osmotic diuretic mannitol or hypertonic saline can establish an osmotic gradient between plasma and brain cells and draws water across the BBB into the vascular compartment. Exemplary dosages for mice were described in the working examples below. The human equivalent doses (HED) can be obtained using methods known in the art. See e.g., Nair AB, Jacob S. J Basic Clin Pharm. 2016 Mar;7(2):27-31. doi: 10.4103/0976-0105.177703 and the FDA's Guidance for Industry. Estimating the Maximum Safe Starting Dose in Initial Clinical Trials for Therapeutics in Adult Healthy. For example, to a human subject, NaCl may be administered at 30 mg/kg or more (e.g., 30 to 300 mg/kg) and mannitol may be administered at 130 mg/kg or more (e.g., 130 to 1300 mg/kg).

FIG. 16 shows a three-compartment model of the relationship between blood plasma, brain, and CSF under isotonic and hypertonic conditions. In the situation of an isotonic blood plasma, there is no change in interstitial fluid volume (V_{ISF}; brain water content, BWC) or pressure (P_{ISF}; intracranial pressure, ICP), and as a result there is no change in the net direction or magnitude of glymphatic flow. In the hypertonic condition, with increased plasma osmolyte content there will be a net resorption of ISF, resulting in decreased ISF volume, and a negative ISF pressure that will enhance CSF influx into brain.

In fact, as shown in the examples below, hyperosmolar therapy with e.g., intraperitoneal hypertonic saline or mannitol, doubled the penetration of an intracisternally-delivered CSF tracer, while increasing influx speeds by about 70%. This response is attributed to an increase in ISF-to-plasma efflux, causing a decrease in ICP without BBB disruption. Accordingly, the same approach can be used to improve the delivery of any composition or compound of interest into the CNS interstitium.

In some embodiments, a composition or compound to be delivered (e.g., therapeutic composition or an imaging composition) is administered intracisternally or intrathecally. Other routes of administration (e.g., parenteral delivery, intravenous delivery, intradermal, or intramuscular intramuscular) can also be used. In that case, the composition or compound will need to cross the BBB. In that case, various means known in the art can be used to facilitate BBB crossing. See, e.g., U.S. Pat. No. 9,675,849, U.S. Pat. No. 7,943,129, US20180134797, US20180237496, and US 20170145076.

For example, the composition or compound can be modified, linked, or conjugated with polypeptides that bind to a BBB receptor and are capable of being transported across the BBB. BBB receptors are expressed on BBB endothelia, as well as other cell and tissue types. Binding of a polypeptide to the BBB receptor can initiate internalization of the polypeptide and transport across the BBB. Such receptors include, but are not limited to, TMEM30A, transferrin receptor (TfR), insulin receptor, insulin-like growth factor receptor (IGF-R), low density lipoprotein receptor (LDLR), low density lipoprotein receptor-related protein 1 (LRP1), low density lipoprotein receptor-related protein 2 (LRP2), low density lipoprotein receptor-related protein 8 (LRP8), GLUT1, basigin, diphtheria toxin receptor, membrane-bound precursor of heparin binding epidermal growth factor-like growth factor (HB-EGF), melanotransferrin, and vasopressin receptor.

In the case where the compound is an antibody, certain domains of the antibody (e.g., the Fc region or one of the antigen-binding domain) can be modified to generate a mutant Fc region or a bi-specific antibody capable of binding to a blood-brain barrier receptor. U.S. Pat. No. 9,676,849, US20180134797, and US20180237496.

Non-polypeptide compounds may also be joined to a BBB receptor-binding polypeptide. Such agents include cytotoxic agents, imaging agents, DNA or RNA molecules, or small molecule compounds. In some embodiments, the compound is a small molecule, e.g., less than 1000 Da, less than 750 Da, or less than 500 Da.

A compound, either a polypeptide or non-polypeptide, may be joined to the N-terminal or C-terminal region of the BBB receptor-binding polypeptide, or attached to any region of the polypeptide, so long as the compound does not interfere with binding of the BBB-receptor binding polypeptide to the BBB receptor. In various embodiments, the conjugates can be generated using well-known chemical cross-linking reagents and protocols. For example, there are a large number of chemical cross-linking agents that are known to those skilled in the art and useful for cross-linking the polypeptide with a compound of interest.

### Nanoparticles

The hyperosmolality-mediated CSF influx described above may be used for delivery of nanoparticles. As used herein, a “nanoparticle” means a particle having a maximum characteristic size of less than 1 micron. There are no limitations on the nanoparticles of this disclosure. Preferably, they can encapsulate an additional agent or associate, covalently or non-covalently, with the agent. Additionally, the nanoparticles may preferably exhibit in vitro and in vivo stability.

The composition, size and shape of the nanoparticle are not particularly limited. For example, for many administration routes, the nanoparticle may be a lipid based particle, such as a liposome, a micelle or lipid encapsulated perfluorocarbon emulsion; an ethosome; a carbon nanotube, such as single wall carbon nanotube; a fullerene nanoparticle; a metal nanoparticle, such gold nanoparticle or silver nanoparticle; a semiconductor nanoparticle, such as quantum dot or boron doped silicon nanowire; a polymer nanoparticle, such as particles made of biodegradable polymers and ion doped polyacrylamide particles; an oxide nanoparticle, such as iron oxide particle, a polymer coated iron oxide nanoparticle or a silicon oxide particle; a viral particle, such as an engineered viral particle or an engineered virus-polymer particle; a polyionic particle, such as leashed polycations; a ceramic particle, such as silica based ceramic nanoparticles, or a combination thereof.

Nanoparticle disclosed herein can have a variety of different particle sizes, depending on the exact target tissue. In some embodiments, the nanoparticles can have an average particle size (d50) from about 1-1,000 nm, from about 100-1,000 nm, from about 100-900 nm, from about 100-800 nm, from about 100-700 nm, from about 100-600 nm, from about 100-500 nm, from about 100-400 nm, from about 100-300 nm, from about 100-200 nm, from about 1-100 nm, from about 2-100 nm, from about 5-50 nm, or from about 10-30 nm.

A variety of different agents can be included in, attached to, conjugated to, or coated to the nanoparticles. In some instances, the agent is a therapeutic agent (e.g., a therapeutic protein, peptide, small molecule, aptamer, or nucleic acid), while in other instances the agent has a diagnostic purpose, for instance a tracer element (e.g., a dye, a radionuclide, contrast agent, and the like). A preferred agent is a therapeutic protein, which may include PEGylated proteins, antibodies, and monoclonal antibodies. The therapeutic protein can have a variety of different molecular weights. For instance, the therapeutic protein can have a molecular weight between about 10,000 Da and 100,000 kDa.

In some embodiments, the nanoparticle can be configured to target a particular target site in a body of the subject. For example, the surface of the nanoparticle may have one or more antibodies that may conjugate with surface marker antigens of certain types of cells. Thus, the nanoparticle may selectively target cells that carry such marker antigens. The examples of cells that carry surface marker antigens include stem or clonogenic cells and tumor cells. A number of monoclonal antibodies to tumor specific antigens are available, see, e.g., pp. 301-323 of CANCER, 3rd Ed., De Vita, et. al. eds; Janeway et. al. Immunology 5th Edition, Garland Press, New York, 2001; A. N. Nagappa, D. Mukheijee & K. Anusha “Therapeutic Monoclonal Antibodies”, PharmaBiz.com, Wednesday, Sep. 22, 2004. Table 2 presents FDA approved monoclonal antibodies for treatment of cancer.

In some embodiments, the surface of the nanoparticle can have hydrophilic polymer chains, such as PEG chains, disposed on it. In some embodiments, the surface of the nanoparticle can be modified, for example, to facilitate the nanoparticle's ability to reach its target site. The surface modification may include a chemical modification, or electrostatic modification, or both. Techniques for the chemical and/or electrostatic surface modification of nanoparticle are known in the art.

Nanoparticles have considerable potential for diagnosis and treatment of CNS disorders, but the BBB limits their CNS access. Although direct cerebrospinal fluid (CSF) administration bypasses the BBB, this approach has shown poor tissue uptake of nanoparticles. As disclosed herein, this disclosure presents efficient CNS distribution of intrathecally administered nanoparticles, such as ¹¹¹In-radiolabeled small gold nanoparticles (AuNPs) in dynamic whole-body single-photon emission tomography (SPECT). As shown in the examples, small AuNPs (10-15 nm) were used in combination with systemic hypertonic saline, a clinically available intervention that accelerates CSF influx, to dramatically increase the uptake of AuNPs especially in deep brain regions. AuNPs entered the brain along the periarterial glymphatic route as visualized by magnetic resonance imaging of gadolinium-labelled AuNPs. AuNPs were largely cleared from the CNS within 24 hours and excreted through the kidneys. Thus, the glymphatic perivascular network combined with transient increases in plasma osmolarity is a novel route for highly efficient brain-wide distribution of small AuNPs.

Novel efficacious treatments for disorders affecting the CNS are urgently needed due to their steeply rising human and societal costs. Nanoparticles are a promising solution to improve the safety and efficacy of drugs targeting the CNS. The size of nanoparticles typically can range from 1 to 100 nanometres, and their structure and composition can be modified to influence their pharmacokinetics, and ultimately that of their drug cargo. Nanoparticles can improve drug stability, target accumulation and exposure duration, thus increasing the therapeutic effect. Indeed, nanoparticles can be used to deliver a variety of drugs, including biological macromolecules, genes, vaccines, proteins, hydrophobic and hydrophilic drugs.

While nanoparticles possess many intriguing features, their size and surface properties makes their passage across the BBB particularly challenging. Even the most intricate nanoparticles designed to cross the BBB have resulted in restricted brain uptake, generally in the range of 0.1%. Administering drugs directly to the cerebrospinal fluid by intrathecal injection circumvents the BBB, and it has been proposed to be particularly useful for nanoparticles, which are not as easily cleared from the CNS compared with free small-molecular drugs. However, the diffusive penetration of nanoparticles and other macromolecules from the subarachnoid space to deep CNS structures has been considered limited due to their large size.

Findings on the glymphatic system add an exciting dimension to improving the CNS delivery of intrathecally administered nanoparticles. The glymphatic pathway is a physiologically modulated CNS-wide fluid transport system. It facilitates the flow of CSF in the periarterial spaces of penetrating arteries into the deep regions of the brain and through the brain parenchyma to clear the brain interstitium of metabolic waste. Compared with the awake state, glymphatic CSF transport dramatically increases during natural slow-wave sleep and under certain anesthetic regimens. In addition, transient increase in plasma osmolarity by systemically administered mannitol or hypertonic saline was shown to transiently boost periarterial CSF influx. Hypertonic treatment enhanced binding of an intracisternally administered amyloid-β antibody to amyloid plaques in mice, suggesting that hypertonic solutions could be utilized as adjuvants to improve CNS distribution of large intrathecally delivered drugs.

Here, as shown in the examples below, small gold nanoparticles (AuNPs) labelled with indium-111 (¹¹¹In) or gadolinium (Gd) were administered to the intrathecal space of rats and imaged their distribution using SPECT or magnetic resonance imaging (MRI), respectively. It was demonstrated that hypertonic saline enhances the global CNS delivery of intrathecal AuNPs within periarterial spaces of penetrating arteries, leading to increased intracranial exposure and a several-fold increase in the distribution of AuNPs to deep brain structures. The small AuNPs were cleared from the brain by lymphatic structures and excreted through the kidneys, rendering their accumulation elsewhere in the body, in particular the liver, insignificant. Thus, glymphatic-assisted intrathecal delivery is a novel strategy for the widescale delivery of nanoparticles, such as AuNPs, to the brain and the spinal cord.

This disclosure provides a new brain-wide drug delivery strategy with nanoparticles, such as small nanoparticles. In some embodiments, small AuNPs are used for three reasons: first, they are expected to retain their size and shape in vivo (Alkilany et al., Acc Chem Res 46, 650-661 (2013), second, they are smaller than the assumed gap between astrocyte endfeet (Iliff, J. J. et al. A paravascular pathway facilitates CSF flow through the brain parenchyma and the clearance of interstitial solutes, including amyloid β. Sci Transl Med 4, 147ra111 (2012)), enabling them to penetrate from the perivascular spaces to the neuropil, and third, they can be renally excreted after their egress from the CNS (Longmire et al., Nanomedicine (Lond) 3, 703-717 (2008)).

As demonstrated in the examples shown herein, after intrathecal administration, small AuNPs can be distributed widely in the CSF space. Transient enhancement of periarterial glymphatic flow by systemic hypertonic saline enhanced the CNS exposure to AuNPs and markedly improved the penetration of AuNPs into the deep brain structures as demonstrated by the elevenfold increase in the thalamic availability of AuNPs during the first three hours. Although nearly all AuNPs had been cleared from the CNS, the difference between the hypertonic and isotonic groups still persisted at 24 hours. These findings suggest that facilitating perivascular glymphatic flow by hypertonic saline is an effective strategy for delivering small nanoparticles into deep brain regions using a relatively non-invasive and safe approach.

Distribution of intrathecally administered nanoparticles have previously been mainly studied using ex vivo techniques and large nanoparticles. The dynamic in vivo SPECT and MRI imaging approaches disclosed herein overcome the limits of ex vivo methods, such as perfusion fixation and euthanasia, that cause rapid marked perivascular CSF influx (Ma, Q. et al. Acta Neuropathol. 137, 151-165 (2019) and Du, T. et al. Cerebrospinal fluid is a significant fluid source for anoxic cerebral oedema. Brain (2021)), and thereby possibly overestimate the parenchymal distribution of tracers. The nanoparticles used in previous reports have been ten times larger compared with this study. Householder and colleagues administered 122 nm PEGylated polystyrene nanoparticles into the cisterna magna of mice and observed wide distribution in the subarachnoid space but no entry into deeper brain structures (Householder et al., Sci Rep 9, 12587-11 (2019)). Likewise, Dengler et al. reported no deep brain distribution for mesoporous silica nanoparticles of 230 nm (Dengler et al. J Control Release 168, 209-224 (2013)). Two studies report nanoparticle-mediated delivery of siRNA into the parenchyma without quantifying the accumulation of nanoparticles (Shyam, R. et al. Mol Ther Nucleic Acids 4, e242 (2015) and Hagihara et al. Gene Ther. 7,759-763 (2000)). Thus, the literature suggests that parenchymal entry of intrathecally administered large nanoparticles is not feasible.

A nanoparticle delivery strategy described herein takes advantage of the finding that simultaneous systemic hypertonic saline transiently boosts glymphatic delivery of nanoparticles. In one example, inventors administered 40 mOsm kg⁻¹ of HTS which is within the same order of magnitude to doses used in clinical trials and practice (Strandvik et al., Anaesthesia 64, 990-1003 (2009)). Surprisingly, the effect of HTS compared with the isotonic group persisted at 24 hours, suggesting HTS influences drug availability longer compared with its effect on intracranial pressure (Shi et al., Medicine (Baltimore) 99, e21655 (2020)). HTS has been used in various clinical contexts, most often to treat elevated intracranial pressure or to restore blood pressure in shock (Strandvik et al., Anaesthesia 64, 990-1003 (2009)). Despite marked elevations in plasma osmolality or sodium levels, these trials have not reported serious adverse effects related to the use of hypertonic saline (Shi et al., Medicine (Baltimore) 99, e21655 (2020)). The most severe potential adverse effect is central pontine demyelinolysis which threatens the malnourished or hyponatremic individuals whose sodium is corrected too rapidly (Shi et al., Medicine (Baltimore) 99, e21655 (2020)). In healthy volunteers, intravenous infusion of 10 mOsm kg⁻¹ of HTS over 30 minutes led to a rapid increase in plasma volume and elevated serum sodium levels, with no reported adverse effects (Järvelä, et al., Anaesthesia 58, 878-881 (2003)). Thus, the systemic hypertonic intervention is an available and safe intervention that should be further characterized as an adjunct to intrathecally administered therapeutics in CNS drug delivery.

Another advantage of the approach described herein is the optimized anesthetic regimen. In one example, one can use a anesthesia composition comprising ketamine and dexmedetomidine as their use improves glymphatic uptake of tracers into the brain in rodents. Further, the high α₂-receptor selectivity and slow-wave inducing properties of dexmedetomine should theoretically be superior to ketamine-xylazine anesthesia that is another frequently used regimen reported to increase glymphatic CSF influx compared with inhaled anesthetics or the awake state.

The weight ratio of ketamine to dexmedetomidine in the composition can range from 100:1 to 400: 1, such as 150:1, 200:1, and 300:1. Administration of the anesthesia composition to achieve anesthesia effect may be via any suitable parenteral route, e.g., intravenous, intrathecal, epidural, caudal transdermal, intradermal, transmucosal, subcutaneous, topical, interscalene, intradiscal, periodontal, intramuscular administration or via a respiratory pathway (e.g., suitably developed for inhalational, pulmonary, and intranasal). Certain clinical situations may require administration of the present composition as a single effective dose, or may be administered as multiple doses or multiple locations.

The anesthesia composition can be administered at a dose sufficient to achieve a desired anesthetic endpoint, for example, immobility, amnesia, analgesia, unconsciousness or autonomic quiescence. Administered dosages for anesthetic agents may be in accordance with dosages and scheduling regimens practiced by those of skill in the art. General guidance for appropriate dosages of pharmacological agents used in the present methods is provided in Goodman and Gilman's The Pharmacological Basis of Therapeutics, 12^{th} Edition, 2010, supra, and in a Physicians' Desk Reference (PDR), for example, in the 65^{th} (2011) or 66^{th} (2012) Eds., PDR Network, each of which is hereby incorporated herein by reference. The appropriate dosage of anesthetic agents will vary according to several factors, including the chosen route of administration, the formulation of the composition, subject/patient response, the severity of the condition, the subject's weight, and the judgment of the prescribing physician. The dosage can be increased or decreased over time, as required by an individual subject or patient. Usually, a subject or patient initially is given a low dose, which is then increased to an efficacious dosage tolerable to the subject or patient.

It will be understood by those having ordinary skill in the art that the specific dose level and frequency of dosage for any particular subject or patient may be varied and will depend upon many factors including the activity of the specific compound employed, the metabolic stability and length of action of that compound, the age, body weight, general health, gender, diet and the particular condition of a subject being administered.

In certain embodiments, nanoparticles, such as AuNPs, can be used to deliver a variety of drugs relevant for diseases affecting the CNS. In general, AuNPs may be surface functionalized through carboxylates or the formation of Au—S bonds that are known to exhibit sufficient stability in vivo (Spadavecchia et al. Int J Nanomedicine 11, 791-822 (2016)). Accordingly, desired compounds may be attached directly to the AuNP surface, or conjugated to other substances, such as PEG-thiol polymers or lipoic acid, that are in turn attached to the AuNP surface. Such modifications can be used to prepare AuNPs decorated with antibodies for receptor targeting, oligonucleotides for gene silencing, and release systems for small molecular drugs. These various modification possibilities make AuNPs highly relevant for therapies targeting the brain.

Due to the extensive network of perivascular spaces in the brain, the glymphatic-enhanced AuNP brain delivery strategy can be particularly valuable in the treatment of diffuse diseases affecting the brain, such as brain cancers involving both hemispheres. Indeed, nanoparticles may be particularly useful in cancer as they can be modified to function as theranostic agents with the dual function of imaging and therapy packed in one nanoparticle (Tang et al. Emerging blood-brain-barrier-crossing nanotechnology for brain cancer theranostics. Chem Soc Rev 48, 2967-3014 (2019)) and to overcome cancer drug resistance (Markman et al. Adv. Drug Deliv. Rev. 65, 1866-1879 (2013)). As a relatively non-selective transport route, glymphatic-enhanced transport can be harnessed for the delivery of both simple, non-targeted and more intricate functionalized nanoparticle designs. In addition to nanoparticles described herein, other types nanoparticles can also be used with the glymphatic delivery approach described herein.

This approach of circumventing the blood-brain barrier through intrathecal administration is superior to systemic chemotherapy targeting the whole brain, as the blood-brain barrier hampers the penetration of most compounds from the systemic circulation into the brain (Wolak et al. Mol. Pharmaceutics 10, 1492-1504 (2013), Banks, W. A. Nat Rev Drug Discov 15, 275-292 (2016), and Terstappen et al. Nat Rev Drug Discov 20, 362-383 (2021)). Accordingly, therapeutics can be administered with intrathecal administration, for instance in pain management, antimicrobial administration, and chemotherapy in the manner described in e.g., Bruel et al., Pain. Pain Med 17, 2404-2421 (2016), Ng et al. Neurocrit Care 20, 158-1712014), and Ruggiero et al. Paediatr Drugs 3, 237-246 (2001). The lumbar intrathecal route is the most frequently used technique, and safe according to a large survey from Sweden that reported a complications rate of 1 per 20-30,000 after intrathecal anesthesia (Moen et al. Anesthesiology 101, 950-959 (2004)).

This disclosure for the first time demonstrated that the brain-wide availability of nanoparticles, such as small gold nanoparticles, can be increased with systemic hypertonic saline up to 24 hours in vivo. Further, the nanoparticles were rapidly eliminated from the body after leaving the CNS. As the astrocytic gaps in the endfeet are most likely a significant limiting factor for drug delivery to the neuropil, the use of different sizes of nanoparticles can be a useful tool to indirectly assess the astrocytic gap width in vivo. Future studies can be carried out to characterize the optimal size and properties of nanoparticles for glymphatic intrathecal delivery. As systemic hypertonic saline has been studied in healthy volunteers and is already in clinical use, clinical trials should assess glymphatic-enhanced drug delivery on intrathecally administered therapeutics.

### Therapeutic Methods

The hyperosmolality-mediated CSF influx described above may be used in therapeutic methods. In some aspects, the invention provides a method of transporting a therapeutic composition or compound into CNS of a patient or subject. The patient or subject can be one having a neurological disorder, including, without limitation: Alzheimer's disease (AD), stroke, dementia, muscular dystrophy (MD), multiple sclerosis (MS), amyotrophic lateral sclerosis (ALS), cystic fibrosis, Angelman's syndrome, Liddle syndrome, Parkinson's disease, Pick's disease, Paget's disease, cancer, traumatic brain injury, etc.

In some embodiments, the neurological disorder is selected from: a neuropathy, an amyloidosis, cancer (e.g. involving the CNS or brain), an ocular disease or disorder, a viral or microbial infection, inflammation (e.g. of the CNS or brain), ischemia, neurodegenerative disease, seizure, behavioral disorder, lysosomal storage disease, etc. The methods of the invention are particularly suited to treatment of such neurological disorders due to their ability to transport one or more associated active ingredients or therapeutic compounds into the CNS/brain where such disorders find their molecular, cellular, or viral/microbial basis.

Neuropathy disorders are diseases or abnormalities of the nervous system characterized by inappropriate or uncontrolled nerve signaling or lack thereof, and include, but are not limited to, chronic pain (including nociceptive pain), pain caused by an injury to body tissues, including cancer-related pain, neuropathic pain (pain caused by abnormalities in the nerves, spinal cord, or brain), and psychogenic pain (entirely or mostly related to a psychological disorder), headache, migraine, neuropathy, and symptoms and syndromes often accompanying such neuropathy disorders such as vertigo or nausea.

For a neuropathy disorder, a neurological drug may be selected that is an analgesic including, but not limited to, a narcotic/opioid analgesic (i.e., morphine, fentanyl, hydrocodone, meperidine, methadone, oxymorphone, pentazocine, propoxyphene, tramadol, codeine and oxycodone), a nonsteroidal anti-inflammatory drug (NSAID) (i.e., ibuprofen, naproxen, diclofenac, diflunisal, etodolac, fenoprofen, flurbiprofen, indomethacin, ketorolac, mefenamic acid, meloxicam, nabumetone, oxaprozin, piroxicam, sulindac, and tolmetin), a corticosteroid (i.e., cortisone, prednisone, prednisolone, dexamethasone, methylprednisolone and triamcinolone), an anti-migraine agent (i.e., sumatriptin, almotriptan, frovatriptan, sumatriptan, rizatriptan, eletriptan, zolmitriptan, dihydroergotamine, eletriptan and ergotamine), acetaminophen, a salicylate (i.e., aspirin, choline salicylate, magnesium salicylate, diflunisal, and salsalate), an anti-convulsant (i.e., carbamazepine, clonazepam, gabapentin, lamotrigine, pregabalin, tiagabine, and topiramate), an anaesthetic (i.e., isoflurane, trichloroethylene, halothane, sevoflurane, benzocaine, chloroprocaine, cocaine, cyclomethycaine, dimethocaine, propoxycaine, procaine, novocaine, proparacaine, tetracaine, articaine, bupivacaine, carticaine, cinchocaine, etidocaine, levobupivacaine, lidocaine, mepivacaine, piperocaine, prilocaine, ropivacaine, trimecaine, saxitoxin and tetrodotoxin), and a cox-2-inhibitor (i.e., celecoxib, rofecoxib, and valdecoxib). For a neuropathy disorder with vertigo involvement, a neurological drug may be selected that is an anti-vertigo agent including, but not limited to, meclizine, diphenhydramine, promethazine and diazepam. For a neuropathy disorder with nausea involvement, a neurological drug may be selected that is an anti-nausea agent including, but not limited to, promethazine, chlorpromazine, prochlorperazine, trimethobenzamide, and metoclopramide.

Amyloidoses are a group of diseases and disorders associated with extracellular proteinaceous deposits in the CNS, including, but not limited to, secondary amyloidosis, age-related amyloidosis, Alzheimer's Disease (AD), mild cognitive impairment (MCI), Lewy body dementia, Down's syndrome, hereditary cerebral hemorrhage with amyloidosis (Dutch type); the Guam Parkinson-Dementia complex, cerebral amyloid angiopathy, Huntington's disease, progressive supranuclear palsy, multiple sclerosis; Creutzfeld Jacob disease, Parkinson's disease, transmissible spongiform encephalopathy, HIV-related dementia, amyotropic lateral sclerosis (ALS), inclusion-body myositis (IBM), and ocular diseases relating to beta-amyloid deposition (i.e., macular degeneration, drusen-related optic neuropathy, and cataract).

For amyloidosis, a neurological drug may be selected that includes, but is not limited to, an antibody or other binding molecule (including, but not limited to a small molecule, a peptide, an aptamer, or other protein binder) that specifically binds to a target selected from: beta secretase, tau, presenilin, amyloid precursor protein or portions thereof, amyloid beta peptide or oligomers or fibrils thereof, death receptor 6 (DR6), receptor for advanced glycation endproducts (RAGE), parkin, and huntingtin; a cholinesterase inhibitor (i.e., galantamine, donepezil, rivastigmine and tacrine); an NMDA receptor antagonist (i.e., memantine), a monoamine depletor (i.e., tetrabenazine); an ergoloid mesylate; an anticholinergic antiparkinsonism agent (i.e., procyclidine, diphenhydramine, trihexylphenidyl, benztropine, biperiden and trihexyphenidyl); a dopaminergic antiparkinsonism agent (i.e., entacapone, selegiline, pramipexole, bromocriptine, rotigotine, selegiline, ropinirole, rasagiline, apomorphine, carbidopa, levodopa, pergolide, tolcapone and amantadine); a tetrabenazine; an anti-inflammatory (including, but not limited to, a nonsteroidal anti-inflammatory drug (i.e., indomethicin and other compounds listed above); a hormone (i.e., estrogen, progesterone and leuprolide); a vitamin (i.e., folate and nicotinamide); a dimebolin; a homotaurine (i.e., 3-aminopropanesulfonic acid; 3APS); a serotonin receptor activity modulator (i.e., xaliproden); an, an interferon, and a glucocorticoid.

Cancers of the CNS are characterized by aberrant proliferation of one or more CNS cell (i.e., a neural cell) and include, but are not limited to, glioma, glioblastoma multiforme, meningioma, astrocytoma, acoustic neuroma, chondroma, oligodendroglioma, medulloblastomas, ganglioglioma, Schwannoma, neurofibroma, neuroblastoma, and extradural, intramedullary or intradural tumors. For cancer, a neurological drug may be selected that is a chemotherapeutic agent.

Examples of chemotherapeutic agents include alkylating agents such as thiotepa and CYTOXANO cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphor-amide and trimethylolomelamine; acetogenins (especially bullatacin and bullatacinone); delta-9-tetrahydrocannabinol (dronabinol, MARINOL); beta-lapachone; lapachol; colchicines; betulinic acid; a camptothecin (including the synthetic analogue topotecan (HYCAMTIN), CPT-11 (irinotecan, CAMPTOSAR), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e. g., calicheamicin, especially calicheamicin gammall and calicheamicin omegall (see, e.g., Agnew, Chem Intl. Ed. Engl., 33: 183-186 (1994)); dynemicin, including dynemicin A; an esperamicin; as well as neocarzinostatin chromophore and related chromoprotein enediyne antiobiotic chromophores), aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, ADRIAMYCIN. doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK. polysaccharide complex (JHS Natural Products, Eugene, Oreg.); razoxane; rhizoxin; sizofiran; spirogermanium; tenuazonic acid; triaziquone; 2,2′, 2″-trichlorotriethylamine; trichothecenes (especially T-2 toxin, verracurin A, roridin A and anguidine); urethan; vindesine (ELDISINEO, FILDESIN); dacarbazine; mannomustine; mitobronitol; mitolactol; pipobroman; gacytosine; arabinoside (“Ara-C”); thiotepa; taxoids, e.g., TAXOL paclitaxe), ABRAXANE Cremophor-free, albumin-engineered nanoparticle formulation of paclitaxel (American Pharmaceutical Partners, Schaumberg, Ill.), and TAXOTERE doxetaxel (Rhone-Poulenc Rorer, Antony, France); chloranbucil; gemcitabine (GEMZAR); 6-thioguanine; mercaptopurine; methotrexate; platinum analogs such as cisplatin and carboplatin; vinblastine (VELBAN); platinum; etoposide (VP-16); ifosfamide; mitoxantrone; vincristine (ONCOVIN); oxaliplatin; leucovovin; vinorelbine (NAVELBINE); novantrone; edatrexate; daunomycin; aminopterin; ibandronate; topoisomerase inhibitor RFS 2000; difluorometlhylornithine (DMFO); retinoids such as retinoic acid; capecitabine (XELODA); pharmaceutically acceptable salts, acids or derivatives of any of the above; as well as combinations of two or more of the above such as CHOP, an abbreviation for a combined therapy of cyclophosphamide, doxorubicin, vincristine, and prednisolone, and FOLFOX, an abbreviation for a treatment regimen with oxaliplatin (ELOXATIN) combined with 5-FU and leucovovin.

Also included in this definition of chemotherapeutic agents are anti-hormonal agents that act to regulate, reduce, block, or inhibit the effects of hormones that can promote the growth of cancer, and are often in the form of systemic or whole-body treatment. They may be hormones themselves.

Another group of compounds that may be selected as neurological drugs for cancer treatment or prevention are anti-cancer immunoglobulins (including, but not limited to, trastuzumab, pertuzumab, bevacizumab, alemtuxumab, cetuximab, gemtuzumab ozogamicin, ibritumomab tiuxetan, panitumumab and rituximab). In some instances, antibodies in conjunction with a toxic label or conjugate may be used to target and kill desired cells (i.e., cancer cells), including, but not limited to, tositumomab with a ¹³¹1 radiolabel, or trastuzumab emtansine.

Viral or microbial infections of the CNS include, but are not limited to, infections by viruses (i.e., influenza, HIV, poliovirus, rubella,), bacteria (i.e., Neisseria sp., Streptococcus sp., Pseudomonas sp., Proteus sp., E. coli, S. aureus, Pneumococcus sp., Meningococcus sp., Haemophilus sp., and Mycobacterium tuberculosis) and other microorganisms such as fungi (i.e., yeast, Cryptococcus neoformans), parasites (i.e., toxoplasma gondii) or amoebas resulting in CNS pathophysiologies including, but not limited to, meningitis, encephalitis, myelitis, vasculitis and abscess, which can be acute or chronic.

For a viral or microbial disease, a neurological drug may be selected that includes, but is not limited to, an antiviral compound (including, but not limited to, an adamantane antiviral (i.e., rimantadine and amantadine), an antiviral interferon (i.e., peginterferon alfa-2b), a chemokine receptor antagonist (i.e., maraviroc), an integrase strand transfer inhibitor (i.e., raltegravir), a neuraminidase inhibitor (i.e., oseltamivir and zanamivir), a non-nucleoside reverse transcriptase inhibitor (i.e., efavirenz, etravirine, delavirdine and nevirapine), a nucleoside reverse transcriptase inhibitors (tenofovir, abacavir, lamivudine, zidovudine, stavudine, entecavir, emtricitabine, adefovir, zalcitabine, telbivudine and didanosine), a protease inhibitor (i.e., darunavir, atazanavir, fosamprenavir, tipranavir, ritonavir, nelfinavir, amprenavir, indinavir and saquinavir), a purine nucleoside (i.e., valacyclovir, famciclovir, acyclovir, ribavirin, ganciclovir, valganciclovir and cidofovir), and a miscellaneous antiviral (i.e., enfuvirtide, foscarnet, palivizumab and fomivirsen)), an antibiotic (including, but not limited to, an aminopenicillin (i.e., amoxicillin, ampicillin, oxacillin, nafcillin, cloxacillin, dicloxacillin, flucoxacillin, temocillin, azlocillin, carbenicillin, ticarcillin, mezlocillin, piperacillin and bacampicillin), a cephalosporin (i.e., cefazolin, cephalexin, cephalothin, cefamandole, ceftriaxone, cefotaxime, cefpodoxime, ceftazidime, cefadroxil, cephradine, loracarbef, cefotetan, cefuroxime, cefprozil, cefaclor, and cefoxitin), a carbapenemipenem (i.e., imipenem, meropenem, ertapenem, faropenem and doripenem), a monobactam (i.e., aztreonam, tigemonam, norcardicin A and tabtoxinine-beta-lactam, a beta-lactamase inhibitor (i.e., clavulanic acid, tazobactam and sulbactam) in conjunction with another beta-lactam antibiotic, an aminoglycoside (i.e., amikacin, gentamicin, kanamycin, neomycin, netilmicin, streptomycin, tobramycin, and paromomycin), an ansamycin (i.e., geldanamycin and herbimycin), a carbacephem (i.e., loracarbef), a glycopeptides (i.e., teicoplanin and vancomycin), a macrolide (i.e., azithromycin, clarithromycin, dirithromycin, erythromycin, roxithromycin, troleandomycin, telithromycin and spectinomycin), a monobactam (i.e., aztreonam), a quinolone (i.e., ciprofloxacin, enoxacin, gatifloxacin, levofloxacin, lomefloxacin, moxifloxacin, norfloxacin, ofloxacin, trovafloxacin, grepafloxacin, sparfloxacin and temafloxacin), a sulfonamide (i.e., mafenide, sulfonamidochrysoidine, sulfacetamide, sulfadiazine, sulfamethizole, sulfanilamide, sulfasalazine, sulfisoxazole, trimethoprim, trimethoprim and sulfamethoxazole), a tetracycline (i.e., tetracycline, demeclocycline, doxycycline, minocycline and oxytetracycline), an antineoplastic or cytotoxic antibiotic (i.e., doxorubicin, mitoxantrone, bleomycin, daunorubicin, dactinomycin, epirubicin, idarubicin, plicamycin, mitomycin, pentostatin and valrubicin) and a miscellaneous antibacterial compound (i.e., bacitracin, colistin and polymyxin B)), an antifungal (i.e., metronidazole, nitazoxanide, tinidazole, chloroquine, iodoquinol and paromomycin), and an antiparasitic (including, but not limited to, quinine, chloroquine, amodiaquine, pyrimethamine, sulphadoxine, proguanil, mefloquine, atovaquone, primaquine, artemesinin, halofantrine, doxycycline, clindamycin, mebendazole, pyrantel pamoate, thiabendazole, diethylcarbamazine, ivermectin, rifampin, amphotericin B, melarsoprol, efornithine and albendazole).

Inflammation of the CNS includes, but is not limited to, inflammation that is caused by an injury to the CNS, which can be a physical injury (i.e., due to accident, surgery, brain trauma, spinal cord injury, concussion) and an injury due to or related to one or more other diseases or disorders of the CNS (i.e., abscess, cancer, viral or microbial infection). For CNS inflammation, a neurological drug may be selected that addresses the inflammation itself (i.e., a nonsteroidal anti-inflammatory agent such as ibuprofen or naproxen), or one which treats the underlying cause of the inflammation (i.e., an anti-viral or anti-cancer agent).

Ischemia of the CNS, as used herein, refers to a group of disorders relating to aberrant blood flow or vascular behavior in the brain or the causes therefor, and includes, but is not limited to: focal brain ischemia, global brain ischemia, stroke (i.e., subarachnoid hemorrhage and intracerebral hemorrhage), and aneurysm. For ischemia, a neurological drug may be selected that includes, but is not limited to, a thrombolytic (i.e., urokinase, alteplase, reteplase and tenecteplase), a platelet aggregation inhibitor (i.e., aspirin, cilostazol, clopidogrel, prasugrel and dipyridamole), a statin (i.e., lovastatin, pravastatin, fluvastatin, rosuvastatin, atorvastatin, simvastatin, cerivastatin and pitavastatin), and a compound to improve blood flow or vascular flexibility, including, e.g., blood pressure medications.

Neurodegenerative diseases are a group of diseases and disorders associated with neural cell loss of function or death in the CNS, and include, but are not limited to: adrenoleukodystrophy, Alexander's disease, Alper's disease, amyotrophic lateral sclerosis, ataxia telangiectasia, Batten disease, cockayne syndrome, corticobasal degeneration, degeneration caused by or associated with an amyloidosis, Friedreich's ataxia, frontotemporal lobar degeneration, Kennedy's disease, multiple system atrophy, multiple sclerosis, primary lateral sclerosis, progressive supranuclear palsy, spinal muscular atrophy, transverse myelitis, Refsum's disease, and spinocerebellar ataxia.

For a neurodegenerative disease, a neurological drug may be selected that is a growth hormone or neurotrophic factor; examples include but are not limited to brain-derived neurotrophic factor (BDNF), nerve growth factor (NGF), neurotrophin-4/5, fibroblast growth factor (FGF)-2 and other FGFs, neurotrophin (NT)-3, erythropoietin (EPO), hepatocyte growth factor (HGF), epidermal growth factor (EGF), transforming growth factor (TGF)-alpha, TGF-beta, vascular endothelial growth factor (VEGF), interleukin-1 receptor antagonist (IL-lra), ciliary neurotrophic factor (CNTF), glial-derived neurotrophic factor (GDNF), neurturin, platelet-derived growth factor (PDGF), heregulin, neuregulin, artemin, persephin, interleukins, glial cell line derived neurotrophic factor (GFR), granulocyte-colony stimulating factor (CSF), granulocyte-macrophage-CSF, netrins, cardiotrophin-1, hedgehogs, leukemia inhibitory factor (LIF), midkine, pleiotrophin, bone morphogenetic proteins (BMPs), netrins, saposins, semaphorins, and stem cell factor (SCF).

Seizure diseases and disorders of the CNS involve inappropriate and/or abnormal electrical conduction in the CNS, and include, but are not limited to epilepsy (i.e., absence seizures, atonic seizures, benign Rolandic epilepsy, childhood absence, clonic seizures, complex partial seizures, frontal lobe epilepsy, febrile seizures, infantile spasms, juvenile myoclonic epilepsy, juvenile absence epilepsy, Lennox-Gastaut syndrome, Landau-Kleffner Syndrome, Dravet's syndrome, Otahara syndrome, West syndrome, myoclonic seizures, mitochondrial disorders, progressive myoclonic epilepsies, psychogenic seizures, reflex epilepsy, Rasmussen's Syndrome, simple partial seizures, secondarily generalized seizures, temporal lobe epilepsy, toniclonic seizures, tonic seizures, psychomotor seizures, limbic epilepsy, partial-onset seizures, generalized-onset seizures, status epilepticus, abdominal epilepsy, akinetic seizures, autonomic seizures, massive bilateral myoclonus, catamenial epilepsy, drop seizures, emotional seizures, focal seizures, gelastic seizures, Jacksonian March, Lafora Disease, motor seizures, multifocal seizures, nocturnal seizures, photosensitive seizure, pseudo seizures, sensory seizures, subtle seizures, sylvan seizures, withdrawal seizures, and visual reflex seizures).

For a seizure disorder, a neurological drug may be selected that is an anticonvulsant or antiepileptic including, but not limited to, barbiturate anticonvulsants (i.e., primidone, metharbital, mephobarbital, allobarbital, amobarbital, aprobarbital, alphenal, barbital, brallobarbital and phenobarbital), benzodiazepine anticonvulsants (i.e., diazepam, clonazepam, and lorazepam), carbamate anticonvulsants (i.e. felbamate), carbonic anhydrase inhibitor anticonvulsants (i.e., acetazolamide, topiramate and zonisamide), dibenzazepine anticonvulsants (i.e., rufinamide, carbamazepine, and oxcarbazepine), fatty acid derivative anticonvulsants (i.e., divalproex and valproic acid), gamma-aminobutyric acid analogs (i.e., pregabalin, gabapentin and vigabatrin), gamma-aminobutyric acid reuptake inhibitors (i.e., tiagabine), gamma-aminobutyric acid transaminase inhibitors (i.e., vigabatrin), hydantoin anticonvulsants (i.e. phenytoin, ethotoin, fosphenytoin and mephenytoin), miscellaneous anticonvulsants (i.e., lacosamide and magnesium sulfate), progestins (i.e., progesterone), oxazolidinedione anticonvulsants (i.e., paramethadione and trimethadione), pyrrolidine anticonvulsants (i.e., levetiracetam), succinimide anticonvulsants (i.e., ethosuximide and methsuximide), triazine anticonvulsants (i.e., lamotrigine), and urea anticonvulsants (i.e., phenacemide and pheneturide).

Behavioral disorders are disorders of the CNS characterized by aberrant behavior on the part of the afflicted subject and include, but are not limited to: sleep disorders (i.e., insomnia, parasomnias, night terrors, circadian rhythm sleep disorders, and narcolepsy), mood disorders (i.e., depression, suicidal depression, anxiety, chronic affective disorders, phobias, panic attacks, obsessive-compulsive disorder, attention deficit hyperactivity disorder (ADHD), attention deficit disorder (ADD), chronic fatigue syndrome, agoraphobia, post-traumatic stress disorder, bipolar disorder), eating disorders (i.e., anorexia or bulimia), psychoses, developmental behavioral disorders (i.e., autism, Rett's syndrome, Aspberger's syndrome), personality disorders and psychotic disorders (i.e., schizophrenia, delusional disorder, and the like).

For a behavioral disorder, a neurological drug may be selected from a behavior-modifying compound including, but not limited to, an atypical antipsychotic (i.e., risperidone, olanzapine, apripiprazole, quetiapine, paliperidone, asenapine, clozapine, iloperidone and ziprasidone), a phenothiazine antipsychotic (i.e., prochlorperazine, chlorpromazine, fluphenazine, perphenazine, trifluoperazine, thioridazine and mesoridazine), a thioxanthene (i.e., thiothixene), a miscellaneous antipsychotic (i.e., pimozide, lithium, molindone, haloperidol and loxapine), a selective serotonin reuptake inhibitor (i.e., citalopram, escitalopram, paroxetine, fluoxetine and sertraline), a serotonin-norepinephrine reuptake inhibitor (i.e., duloxetine, venlafaxine, desvenlafaxine, a tricyclic antidepressant (i.e., doxepin, clomipramine, amoxapine, nortriptyline, amitriptyline, trimipramine, imipramine, protriptyline and desipramine), a tetracyclic antidepressant (i.e., mirtazapine and maprotiline), a phenylpiperazine antidepressant (i.e., trazodone and nefazodone), a monoamine oxidase inhibitor (i.e., isocarboxazid, phenelzine, selegiline and tranylcypromine), a benzodiazepine (i.e., alprazolam, estazolam, flurazeptam, clonazepam, lorazepam and diazepam), a norepinephrine-dopamine reuptake inhibitor (i.e., bupropion), a CNS stimulant (i.e., phentermine, diethylpropion, methamphetamine, dextroamphetamine, amphetamine, methylphenidate, dexmethylphenidate, lisdexamfetamine, modafinil, pemoline, phendimetrazine, benzphetamine, phendimetrazine, armodafinil, diethylpropion, caffeine, atomoxetine, doxapram, and mazindol), an anxiolytic/sedative/hypnotic (including, but not limited to, a barbiturate (i.e., secobarbital, phenobarbital and mephobarbital), a benzodiazepine (as described above), and a miscellaneous anxiolytic/sedative/hypnotic (i.e. diphenhydramine, sodium oxybate, zaleplon, hydroxyzine, chloral hydrate, aolpidem, buspirone, doxepin, eszopiclone, ramelteon, meprobamate and ethclorvynol)), a secretin (see, e.g., Ratliff-Schaub et al. Autism 9: 256-265 (2005)), an opioid peptide (see, e.g., Cowen et al., J. Neurochem. 89:273-285 (2004)), and a neuropeptide (see, e.g., Hethwa et al. Am. J. Physiol. 289: E301-305 (2005)).

Lysosomal storage disorders are metabolic disorders which are in some cases associated with the CNS or have CNS-specific symptoms; such disorders include, but are not limited to: Tay-Sachs disease, Gaucher's disease, Fabry disease, mucopolysaccharidosis (types I, II, III, IV, V, VI and VII), glycogen storage disease, GM1-gangliosidosis, metachromatic leukodystrophy, Farber's disease, Canavan's leukodystrophy, and neuronal ceroid lipofuscinoses types 1 and 2, Niemann-Pick disease, Pompe disease, and Krabbe's disease.

For a lysosomal storage disease, a neurological drug may be selected that is itself or otherwise mimics the activity of the enzyme that is impaired in the disease. Exemplary recombinant enzymes for the treatment of lysosomal storage disorders include, but are not limited to those set forth in e.g., U.S. Patent Application publication no. 2005/0142141 (i.e., alpha-L-iduronidase, iduronate-2-sulphatase, N-sulfatase, alpha-N-acetylglucosaminidase, N-acetyl-galactosamine-6-sulfatase, beta-galactosidase, arylsulphatase B, beta-glucuronidase, acid alpha-glucosidase, glucocerebrosidase, alpha-galactosidase A, hexosaminidase A, acid sphingomyelinase, beta-galactocerebrosidase, beta-galactosidase, arylsulfatase A, acid ceramidase, aspartoacylase, palmitoyl-protein thioesterase 1 and tripeptidyl amino peptidase 1).

The above-described therapeutic composition agent can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein.

Nucleic acid molecules can be administered using techniques known in the art, including via vector, plasmid, liposome, DNA injection, electroporation, gene gun, intravenously injection or hepatic artery infusion. Vectors (including viral vectors) for use in gene therapy embodiments are known in the art.

According to a specific embodiment, the method of the invention allows for water flux from the brain to the blood in a highly controlled manner. The therapeutic composition may be used in combination with a hypertonic solution. Advantageously, the therapeutic composition and hypertonic solution may be administered concurrently at the same time.

### Macroscopic Imaging

In another aspect, this invention provides a novel non-invasive transcranial macroscopic imaging approach that allows one to track cortical CSF flow in real time in the intact brain of a living subject. The method comprises introducing an effective amount of an imaging agent to the central nervous system of a subject, and imaging the brain of the subject. The imaging agent can be delivered intracisternally or intrathecally. In a preferred embodiment, the imaging agent comprises a fluorophore and the step of imaging comprises fluorescence macroscopy. In example, the fluorophore re-emit light in the infrared region (e.g., the near infrared region, the mid infrared region, or the far-infrared region) upon excitation.

As disclosed herein, this new imaging approach exploiting the brain-wide system of perivascular spaces to quickly and effectively enhance delivery of therapeutics. To this end, this invention also provides a novel transcranial optical imaging approach enabling non-invasive and dynamic measurements of CSF transport. With that, one can obtain brain-wide imaging of CSF tracers, in contrast to the narrow field visualized by 2-photon microscopy, while obtaining spatial and temporal resolution that is not attainable with MRI. The high frame rate acquisition is compatible with the use of front-tracking software to quantify CSF transport in the rodent brain by measuring progress of the tracer front at pixel level resolution (56). The macroscope has a large gantry to image small animals in immobilized or behaving configurations (e.g., running wheels or cognitive tests). The placement of non-invasive chronic cyanoacrylate cranial windows enables repeat imaging (57). Accordingly, transcranial optical imaging can be applied to intracerebroventricular or intraparenchymal tracer studies in order to evaluate clearance.

### Kit and Articles of Manufacture

In another aspect of the invention, this invention provides a kit or an article of manufacture containing materials useful for the methods described above. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective (1) for improving delivery of a composition to a central nervous system interstitium, brain interstitium and/or a spinal cord interstitium of a subject or (2) for treating, preventing and/or diagnosing one or more of the conditions mentioned above. The container may have a sterile access port (for example, the container may be an intravenous solution bag or a vial having a stopper pierceable by a hypodermic injection needle). At least one active agent in the composition can be a macromolecule therapeutic, e.g., an antibody. The label or package insert indicates that the composition is used for treating the condition of choice.

Moreover, the article of manufacture may comprise (a) a first container with a composition contained therein, wherein the composition comprises an agent that enhances glymphatic system influx and (b) a second container with a composition contained therein, wherein the composition comprises a therapeutic agent or imaging agent. The article of manufacture in this embodiment of the invention may further comprise a package insert indicating that the compositions can be used to treat a particular condition. Alternatively, or additionally, the article of manufacture may further comprise a third container comprising a pharmaceutically acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

In some embodiments, the kit or article of manufacture further comprises instructional materials containing directions (i.e., protocols) for the practice of the methods described herein (e.g., instructions for using the kit for administering a composition). While the instructional materials typically comprise written or printed materials, they are not limited to such. Any medium capable of storing such instructions and communicating them to an end user is contemplated by this invention. Such media include, but are not limited to, electronic storage media (e.g., magnetic discs, tapes, cartridges, chips), optical media (e.g., CD-ROM), and the like. Such media may include addresses to internet sites that provide such instructional materials.

### Definitions

As used herein, the “osmolality” of a solution is the number of osmoles of solute per kilogram of solvent. Osmolality is a measure of the number of particles present in solution and is independent of the size or weight of the particles. It can be measured only by use of a property of the solution that is dependent only on the particle concentration. These properties are vapour pressure depression, freezing point depression, boiling point elevation, and osmotic pressure, and are collectively referred to as colligative properties. The “osmolarity” of a solution is the number of osmoles of solute per liter of solution.

The terms “polypeptide” and “peptide” are used interchangeably herein to refer to a polymer of amino acid residues in a single chain. The terms apply to amino acid polymers in which one or more amino acid residue is an artificial chemical mimetic of a corresponding naturally occurring amino acid, as well as to naturally occurring amino acid polymers and non-naturally occurring amino acid polymers. Amino acid polymers may comprise entirely L-amino acids, entirely D-amino acids, or a mixture of L and D amino acids. The term “protein” as used herein refers to either a polypeptide or a dimer (i.e., two) or multimer (i.e., three or more) of single chain polypeptides. The single chain polypeptides of a protein may be joined by a covalent bond, e.g., a disulfide bond, or non-covalent interactions.

The term “nucleic acid” refers to deoxyribonucleotides or ribonucleotides and polymers thereof in either single- or double-stranded form, composed of monomers (nucleotides) containing a sugar, phosphate and a base that is either a purine or pyrimidine. Unless specifically limited, the term encompasses nucleic acids containing known analogs of natural nucleotides that have similar binding properties as the reference nucleic acid and are metabolized in a manner similar to naturally occurring nucleotides. A “nucleic acid fragment” is a fraction of a given nucleic acid molecule. The term “nucleotide sequence” refers to a polymer of DNA or RNA that can be single- or double-stranded, optionally containing synthetic, non-natural or altered nucleotide bases capable of incorporation into DNA or RNA polymers. The terms “nucleic acid”, “nucleic acid molecule”, “nucleic acid fragment”, “nucleic acid sequence or segment”, or “polynucleotide” may also be used interchangeably with gene, cDNA, DNA and RNA encoded by a gene.

The term “antibody” herein is used in the broadest sense and encompasses various antibody structures, including but not limited to monoclonal antibodies, polyclonal antibodies, multispecific antibodies (e.g. bispecific antibodies), and antibody fragments so long as they exhibit the desired antigen-binding activity. An “antibody fragment” refers to a molecule other than an intact antibody that comprises a portion of an intact antibody that binds the antigen to which the intact antibody binds. Examples of antibody fragments are well known in the art (see, e.g., Nelson, MAbs (2010) 2(1): 77-83) and include but are not limited to Fab, Fab′, Fab′-SH, F(ab′)₂, and Fv; diabodies; linear antibodies; single-chain antibody molecules including but not limited to single-chain variable fragments (scFv), fusions of light and/or heavy-chain antigen-binding domains with or without a linker (and optionally in tandem); and monospecific or multispecific antigen-binding molecules formed from antibody fragments (including, but not limited to multispecific antibodies constructed from multiple variable domains which lack Fc regions).

“Anti-Aβ antibody” refers to an antibody that specifically binds to human Aβ. A nonlimiting example of an anti-Aβ antibody is crenezumab. Other non-limiting examples of anti-Aβ antibodies are solanezumab, bapineuzumab, gantenerumab, aducanumab, ponezumab and any anti-Abeta antibodies disclosed in the following publications: WO2000162801, WO2002046237, WO2002003911, WO2003016466, WO2003016467, WO2003077858, WO2004029629, WO2004032868, WO2004032868, WO2004108895, WO2005028511, WO2006039470, WO2006036291, WO2006066089, WO2006066171,

WO2006066049, WO2006095041, and WO2009027105.

A “neurological disorder” refers to a disease or disorder which affects the CNS and/or which has an etiology in the CNS. Exemplary CNS diseases or disorders include, but are not limited to, neuropathy, amyloidosis, cancer, an ocular disease or disorder, viral or microbial infection, inflammation, ischemia, neurodegenerative disease, seizure, behavioral disorders, and a lysosomal storage disease. Specific examples of neurological disorders include, but are not limited to, neurodegenerative diseases (including, but not limited to, Lewy body disease, postpoliomyelitis syndrome, Shy-Draeger syndrome, olivopontocerebellar atrophy, Parkinson's disease, multiple system atrophy, striatonigral degeneration, tauopathies (including, but not limited to, Alzheimer disease and supranuclear palsy), prion diseases (including, but not limited to, bovine spongiform encephalopathy, scrapie, Creutzfeldt-Jakob syndrome, kuru, Gerstmann-Straussler-Scheinker disease, chronic wasting disease, and fatal familial insomnia), bulbar palsy, motor neuron disease, and nervous system heterodegenerative disorders (including, but not limited to, Canavan disease, Huntington's disease, neuronal ceroid-lipofuscinosis, Alexander's disease, Tourette's syndrome, Menkes kinky hair syndrome, Cockayne syndrome, Halervorden-Spatz syndrome, lafora disease, Rett syndrome, hepatolenticular degeneration, Lesch-Nyhan syndrome, and Unverricht-Lundborg syndrome), dementia (including, but not limited to, Pick's disease, and spinocerebellar ataxia), cancer (e.g. of the CNS, including brain metastases resulting from cancer elsewhere in the body).

A “neurological disorder drug” is a drug or therapeutic agent that treats one or more neurological disorder(s). Neurological disorder drugs of the invention include, but are not limited to, antibodies, peptides, proteins, natural ligands of one or more CNS target(s), modified versions of natural ligands of one or more CNS target(s), aptamers, inhibitory nucleic acids (i.e., small inhibitory RNAs (siRNA) and short hairpin RNAs (shRNA)), ribozymes, and small molecules, or active fragments of any of the foregoing. Non-limiting examples of neurological disorder drugs and the disorders they may be used to treat are provided herein.

The term “cytotoxic agent” refers to a substance that inhibits or prevents a cellular function and/or causes cell death or destruction. Cytotoxic agents include, but are not limited to, radioactive isotopes (e.g., At²¹¹, I¹³¹, I¹²⁵, Y⁹⁰, Rc¹⁸⁶, Rc¹⁸⁸, Sm¹⁵³, Bi²¹², P³², Pb²¹² and radioactive isotopes of Lu); chemotherapeutic agents or drugs (e.g., methotrexate, adriamicin, vinca alkaloids (vincristine, vinblastine, etoposide), doxorubicin, melphalan, mitomycin C, chlorambucil, daunorubicin or other intercalating agents); growth inhibitory agents; enzymes and fragments thereof such as nucleolytic enzymes; antibiotics; toxins such as small molecule toxins or enzymatically active toxins of bacterial, fungal, plant or animal origin, including fragments and/or variants thereof; and the various antitumor or anticancer agents disclosed herein.

As used herein, an “inhibitory nucleic acid” is a double-stranded RNA, RNA interference, miRNA, siRNA, shRNA, or antisense RNA, or a portion thereof, or a mimetic thereof, that when administered to a mammalian cell results in a decrease in the expression of a target gene. Typically, a nucleic acid inhibitor comprises at least a portion of a target nucleic acid molecule, or an ortholog thereof, or comprises at least a portion of the complementary strand of a target nucleic acid molecule. Typically, expression of a target gene is reduced by 10%, 25%, 50%, 75%, or even 90-100%.

A “therapeutic RNA molecule” or “functional RNA molecule” as used herein can be an antisense nucleic acid, a ribozyme (e.g., as described in U.S. Pat. No. 5,877,022), an RNA that effects spliceosome-mediated trans-splicing (see, Puttaraju et al. (1999) Nature Biotech. 17:246; U.S. Pat. No. 6,013,487; U.S. Pat. No. 6,083,702), an interfering RNA (RNAi) including siRNA, shRNA or miRNA, which mediate gene silencing (see, Sharp et al., (2000) Science 287:2431), and any other non-translated RNA, such as a “guide” RNA and CRISPR RNA (Gorman et al. (1998) Proc. Nat. Acad. Sci. USA 95:4929; U.S. Pat. No. 5,869,248) and the like as are known in the art.

“Anti-sense” refers to a nucleic acid sequence, regardless of length, that is complementary to the coding strand or mRNA of a nucleic acid sequence. Antisense RNA can be introduced to an individual cell, tissue or organanoid. An anti-sense nucleic acid can contain a modified backbone, for example, phosphorothioate, phosphorodithioate, or other modified backbones known in the art, or may contain non-natural internucleoside linkages.

As referred to herein, a “complementary nucleic acid sequence” is a nucleic acid sequence capable of hybridizing with another nucleic acid sequence comprised of complementary nucleotide base pairs. By “hybridize” is meant pair to form a double-stranded molecule between complementary nucleotide bases (e.g., adenine (A) forms a base pair with thymine (T), as does guanine (G) with cytosine (C) in DNA) under suitable conditions of stringency. (See, e.g., Wahl, G. M. and S. L. Berger (1987) Methods Enzymol. 152:399; Kimmel, A. R. (1987) Methods Enzymol. 152:507).

As used herein, the term “siRNA” intends a double-stranded RNA molecule that interferes with the expression of a specific gene or genes post-transcription. In some embodiments, the siRNA functions to interfere with or inhibit gene expression using the RNA interference pathway. Similar interfering or inhibiting effects may be achieved with one or more of short hairpin RNA (shRNA), microRNA (mRNA) and/or nucleic acids (such as siRNA, shRNA, or miRNA) comprising one or more modified nucleic acid residue-e.g. peptide nucleic acids (PNA), locked nucleic acids (LNA), unlocked nucleic acids (UNA), or triazole-linked DNA. Optimally, a siRNA is 18, 19, 20, 21, 22, 23 or 24 nucleotides in length and has a 2-base overhang at its 3′ end. These dsRNAs can be introduced to an individual cell or culture system. Such siRNAs are used to downregulate mRNA levels or promoter activity.

An “imaging agent” is a compound that has one or more properties that permit its presence and/or location to be detected directly or indirectly. Examples of such imaging agents include proteins and small molecule compounds incorporating a labeled moiety that permits detection. An imaging agent can be any chemical or substance that is used to provide the signal or contrast in imaging. Examples include an organic molecule, metal ion, salt or chelate, particle, labeled peptide, protein, polymer or liposome.

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human.

The term “administer” refers to a method of delivering agents, compounds, or compositions to the desired site of biological action. These methods include, but are not limited to, topical delivery, parenteral delivery, intravenous delivery, intradermal delivery, intramuscular delivery, intrathecal delivery, colonic delivery, rectal delivery, or intraperitoneal delivery. In one embodiment, the polypeptides described herein are administered intravenously.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, antibodies of the invention are used to delay development of a disease or to slow the progression of a disease.

An “effective amount” of an agent, e.g., a pharmaceutical formulation, refers to an amount effective, at dosages and for periods of time necessary, to achieve the desired therapeutic or prophylactic result.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. As used herein, the term “pharmaceutical composition” refers to the active agent in combination with a pharmaceutically acceptable carrier commonly used in the pharmaceutical industry.

As used herein, the terms “virus vector,” “vector” or “gene delivery vector” refer to a virus (e.g., AAV) particle that functions as a nucleic acid delivery vehicle, and which comprises the vector genome (e.g., viral DNA [vDNA]) packaged within a virion. Alternatively, in some contexts, the term “vector” may be used to refer to the vector genome/vDNA alone.

As disclosed herein, a number of ranges of values are provided. It is understood that each intervening value, to the tenth of the unit of the lower limit, unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither, or both limits are included in the smaller ranges is also encompassed within the invention, subject to any specifically excluded limit in the stated range. Where the stated range includes one or both of the limits, ranges excluding either or both of those included limits are also included in the invention.

The terms “anesthetic,” “anesthesia,” “anesthesiology” and the like refer herein to substances, compounds, processes or procedures that induce insensitivity to pain such as a temporary loss of sensation.

The term “about” or “approximately” means within an acceptable range for the particular value as determined by one of ordinary skill in the art, which will depend in part on how the value is measured or determined, e.g., the limitations of the measurement system. For example, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Unless otherwise stated, the term “about” means within an acceptable error range for the particular value.

### EXAMPLES

### Example 1 Material and Methods

This example descibes material and methods used in Examples 2-5 bellow.

Animals. For all experiments, male C57BL/6 mice, 8-12 weeks of age (Charles River) were used. Male global aquaporin-4 knockout (Aqp4^{-/-}) mice on a C57BL/6 background, between 8-12 weeks old, were used where indicated (21). Male 6-month-old APP/PS1^{+/-} mice (Jackson Laboratory) were used for the Aβ antibody experiments.

Intracisternal injections. Mice in the KX groups were weighed and anesthetized with a mixture of ketamine (100 mg/kg, i.p.) and xylazine (10 mg/kg, i.p.). Afterwards, animals were fixed in a stereotaxic frame, and the cisterna magna was surgically exposed with the help of a stereomicroscope. The cisternal space was cannulated using a 30 G needle attached via polyethylene tubing to a Hamilton syringe. The needle was secured with cyanoacrylate glue and tracers were infused with a syringe pump (Harvard Apparatus) depending on the experimental paradigm (see below). Mice randomized to the awake group were anesthetized with 2% isoflurane, their skull cemented to a customized head plate, placed in a restraint tube, and then underwent the same surgical procedure as described above. Tracers were allowed to circulate for 30 min after the injection start time and the needle left in place for the duration of the experiment to prevent the CSF compartment from depressurizing. Core temperature (37° C.) and anesthetic depth were maintained throughout the experiment. At the end of 30 min, animals were decapitated, and the brain processed for either fluorescent or radioisotope tracer analysis.

CSF Tracers. AlexaFluor647-conjugated bovine serum albumin (BSA-647, 66 kDa, Invitrogen) was constituted in artificial CSF (0.5% m/v) and used as a fluorescent CSF tracer. Radio-labeled ¹⁴C-inulin (6 kDa, Perkin Elmer) and ³H-dextran (40 kDa, American Radiolabeled Chemicals) were dissolved in artificial CSF at a concentration of 0.1 and 10 μCi/μL, respectively. For APP/PS1 experiments, an AlexaFluor488-conjugated anti-β-amyloid antibody (clone 6E10, 1 mg/mL; BioLegend, Cat. No. 803013) was infused and allowed to circulate for 120 min. Fluorescent tracers and antibodies were infused in a total volume of 10 μl at a rate of 2 μl/min or 1 μl/min into the cisterna magna. Radioisotope tracers were infused in a total volume of 5 μl at 1 μl/min. A direct comparison of 2 μl/min and 1 μl/min infusion rate showed no difference in BSA-647 influx on in vivo imaging (P=0.971, two-way repeated measures ANOVA) or ex vivo coronal sections (P=0.939, unpaired t test).

Transcranial In vivo Macroscopic Imaging. For in vivo imaging, the skin covering the dorsal calvarium was incised and reflected laterally prior to cannulating the cisterna magna. The entry of CSF tracers into the brain was imaged by fluorescence macroscopy (MVX10, Olympus) using a PRIOR Lumen 1600-LED light source and Flash 4.0 digital camera (Hamamatsu). The mouse fixed on the stereotaxic frame was placed on the microscope stage and images at 20× magnification were acquired in the far-red emission channel (647 nm). Images (2048×2048 pixel; 5.7120 μm/pixel) were collected at 1 min intervals for 0-30 min following injection commencement using the MetaMorph Basic imaging software (Molecular Devices). Exposure time was held constant throughout the duration of the imaging sequence and across experimental groups.

Front tracking. To quantify the area and speed of fluorescent CSF tracer influx in the brain, inventors employed an algorithm recently developed in the context of advection-reaction-diffusion (56). Given a time series of a two-dimensional concentration field, this algorithm tracked the location of the “front” which separates low-concentration and high-concentration regions. The algorithm outputs spatially- and temporally-resolved velocity measurements quantifying the front propagation. Influx speed was calculated by averaging over the entire group data to obtain mean front speed measurements for each group. The propagation front was identified using a threshold of 175 (on an 8-bit scale of 0 to 255); however, it was noted that the results were fairly robust to different threshold choices. This same threshold was used for all-time series of images, which was justified since care was taken to maintain similar imaging conditions across all experiments. A fixed threshold preserved the physical meaning of the front and allowed for quantitative comparison between populations from different experiments. More details and a copy of the code, written for MATLAB (MathWorks), are available online (56).

In vivo two-photon imaging. A 3 mm cranial window was placed over the right parietal bone of anesthetized mice. The window was covered with agarose (0.8% at 37° C.) and sealed with a glass coverslip. Imaging was done using a resonant scanner B scope (Thorlabs) with a Chameleon Ultra II laser (Coherent) and a water-immersion 20× objective (1.0 NA, Olympus). Intravascular fluorescein isothiocyanate-dextran (FITC-dextran, 2,000 kDa) was given prior to a CM tracer infusion of bovine serum albumin conjugated to Texas Red (BSA-TxRd, 66 kDa). Z-stacks were taken over the MCA every minute from the start of the infusion for 30 minutes. To measure CSF influx, three circular regions of interest (ROI) were outlined on the perivascular space and measurements for each ROI were taken at every timepoint (ThorImageLS). Fluorescence intensity for all three ROIs were averaged and normalized to the peak fluorescence (ΔF/Fₘₐₓ) and expressed as a percent. Time to tracer appearance was calculated as the first timepoint where fluorescence was above background signal. Orthogonal reconstructions were done using Imaris (Bitplane).

Solutions. Control mice received isosmotic saline (0.34M NaCl in ddH₂O; 20 μL/g, i.p.). Hyperosmolality was induced either with mannitol (1M in 0.34M NaCl; 30 μL/g, i.p.) or hypertonic saline (1M NaCl in ddH₂O; 20 μL/g, i.p.). Thirty minutes after intraperitoneal injection, a plasma sample was taken, and the mouse decapitated. Plasma osmolality was measured in triplicate using a micro-osmometer (Advanced Instruments).

Brain Water Content Measurement. Brains were dissected and immediately weighed (w_{wet}; g). The tissue was dried at 65° C. until they reached a constant weight (˜48 hours). Brains were re-weighed (w_{dry}) and the tissue water content (ml/g dry weight) calculated

$$\frac{{w}_{wet} - {w}_{dry}}{( {w}_{dry} )} .$$

Intracranial and Arterial Blood Pressure Measurements. A separate group of animals were anesthetized with a mixture of ketamine/xylazine. Afterwards, a 30 G needle connected to rigid polyethylene tubing filled with aCSF was inserted into the cisterna magna as described above and an arterial catheter was placed in the femoral artery. The lines were connected to a pressure transducer and monitor (World Precision Instruments). Recordings were allowed to stabilize, and then recorded continuously for 35 min (5 min baseline and 30 min after i.p. injection). The signals were digitized and recorded with a DigiData 1440A digitizer and AxoScope software (Axon Instruments). The intracranial and mean arterial pressure recordings were processed and analyzed using MATLAB (MathWorks).

Laser Doppler Flowmetry. Relative changes in cerebral blood flow (rCBF) were measured using laser Doppler flowmetry (PF5010 Laser Doppler Perfusion Module with microtips, PR 418-1, Perimed). The tip of the fiber optic probe was fixed directly onto the exposed skull with cyanoacrylate glue. Signals were collected using a 1440A digitizer and AxoScope software (Axon Instruments). For each mouse, rCBF was recorded both 5 minutes before and 30 minutes after the administration of i.p. solutions. The rCBF recordings were processed and analyzed using MATLAB (MathWorks).

Assessment of BBB Permeability. For quantification of BBB disruption, a 1% solution of FITC-conjugated dextran (70 kDa; Sigma-Aldrich) in normal saline (4 mL/kg of body weight) was injected via the femoral vein. The dextran was allowed to circulate for 30 min following plasma tonicity manipulations, at which point a plasma sample was taken and the mice were perfusion-fixed as described below. Positive controls received a 2M mannitol infusion via a catheter in the right external carotid artery (0.64 mL/min for 30 seconds) 5 minutes after the dextran. The brains were harvested, sectioned, and FITC extravasation was imaged and quantified (see below). Plasma concentration of the FITC dextran was calculated by diluting the plasma samples 1:4 with PBS and analyzing them in triplicate on a fluorescence microplate reader (SpectraMax M2, Molecular Devices) at 458 nm excitation, 538 nm emission, with a cutoff above 530 nm at room temperature (24° C.). Dextran concentration was estimated by comparing the relative fluorescence of the samples against a standard curve (0.0-1.0 mg/mL, 0.1 mg/mL steps; FITC-dextran 70 kDa in PBS) fitted with a linear regression. Blood samples with hemolysis were excluded from analysis due to interference with spectrophotometric absorbance readings.

Imaging Depth Analysis. The depth of fluorescence detection using macroscopic imaging is highly dependent on the power of the illumination source, excitation/emission wavelength of the fluorophore, and the exposure time. However, inventors attempted to estimate the depth of detection for transcranial optical imaging. For this, anesthetized mice were imaged using the macroscope while receiving an intracisternal infusion of BSA-647 as before. Images were acquired every 60 seconds for a duration of either 5, 10, 15, 20, 25, or 30 minutes. Mice were sacrificed immediately following the corresponding stop time; brains were harvested and drop fixed in 4% PFA overnight and then sectioned (see below). Tracer fluorescence was quantified in coronal sections to determine the depth of fluorescence detection (see Image Analysis). In a separate set of experiments, penetration depth of imaging was calculated for a 635 nm wavelength. To determine the optimal tracer concentration for imaging, BSA-647 was serially diluted (10 to 1×10⁻⁴ mg/ml by increments of 10) and 10 μl were aliquoted into a black 96-well plate. Each well was imaged on the macroscope using the same magnification and exposure time as the in vivo experiments (100 ms). Afterwards, a capillary was loaded with 0.1 mg/ml BSA-647, sealed on both sides, and embedded in a petri dish with 4% agarose, level with the surface. Acute coronal slices of increasing thickness (200-4000 μm) were obtained from control mice and sections were placed over the dye-filled capillary for imaging. The slices were maintained in aCSF during imaging so as to preserve the optical properties of the tissue.

Amyloid-β Plaque Labeling. APP/PS1 mice were injected intraperitoneally with methoxy-X04 (MeX04, Tocris, 10 mg/kg, i.p.) dissolved in DMSO (10%), propylene glycol (45%), and PBS (45%) 24 hours prior to cisterna magna injections, as previously described (59).

Tissue Collection and Processing. For fluorescent CSF tracer influx analysis, mice were decapitated after 30 min of the injection start and the brains drop-fixed in 4% paraformaldehyde (PFA; Sigma-Aldrich) overnight at 4° C. Brains were harvested and fixed within 30 seconds of the completion of image acquisition. For assessment of BBB permeability, mice were transcardially perfused with ice-cold 0.1M PBS (pH 7.4, Sigma-Aldrich) followed by 4% PFA. Brain tissue was carefully dissected away from the skull and dura then post-fixed overnight in 4% PFA at 4° C. For immunohistochemistry, mice were perfusion-fixed as before but FITC-conjugated lectin from Triticum vulgaris (25 μg/mL; Sigma-Aldrich) was added into the ice-cold PBS solution prior to the PFA perfusion step. For the APP/PS1 experiments, mice were lectin-perfused as above but with an AlexaFluor647-conjugated wheat germ agglutinin (WGA) lectin (15 μg/mL; Invitrogen).

Immunohistochemistry. To confirm if CSF tracers entered the brain via para-arterial spaces, coronal slices were stained for AQP4 using a free-float method. Slices were blocked for 1 h at room temperature (7% normal donkey serum, NDS, in 0.5% Triton in PBS) and then incubated with primary rabbit anti-AQP4 (1:500; 1% NDS in 0.1% Triton/PBS, Millipore, Cat. No. AB3594) antibody overnight at 4° C. The sections were then incubated with a secondary Cy3-conjugated donkey anti-rabbit (1:250; Jackson ImmunoResearch Cat. No. 711-165-152) antibody for 2 hours at room temperature and washed. Brain sections were mounted with ProLong Gold Antifade with DAPI (Invitrogen) and allowed to dry for 24 h before imaging.

Ex vivo Fluorescence Imaging. Prior to sectioning, dorsal whole brain images were acquired for CSF tracer (BSA-647) on a stereomicroscope (MVX10, Olympus) at 16× magnification. Afterwards, coronal slices (100 μm thickness) were obtained using a calibrated vibratome (VT1200S, Leica). Beginning at the anterior aspect of the corpus callosum, one section was collected every 5 sections until a total of 6 sections had been acquired for each animal. Brain sections were mounted with ProLong Gold Antifade with DAPI (Invitrogen) and with Fluoromount-G (SouthernBiotech) for MeX04 experiments. The entry of CSF tracer into the brain was evaluated by epifluorescence macroscopy (MVX10, Olympus). Single channel images were acquired with the MetaMorph Basic software (Molecular Devices) at low magnification (20×). Exposures were determined based on control brains and held constant for all groups. To better visualize tracer movement into the brain, inventors imaged coronal slices with a CSF tracer (BSA-647), intravital lectin (FITC), and stained for AQP4 (Cy3) on a laser scanning confocal microscope (IX81, Olympus) using FluoView (FV500, Olympus) software. Multi-channel images from both left and right dorsal cortex were acquired (40 μm z-stacks with 2 μm step size at 40× magnification). In APP/PS1 mice, coronal sections were imaged at 4× magnification using a montage epifluorescence microscope (BX51 Olympus and CellSens Software) and high magnification 20× and 100× images on confocal (Leica SP8 and LASX software).

Image Analysis. All images were analyzed using ImageJ software (National Institutes of Health, imagej.nih.gov/ij/) (60). To measure glymphatic influx in vivo, a customized macro was developed. A region of interest (ROI) was defined based upon the exposed skull perimeter and overlaid on a 31-image (8-bit; 2048×2048 pixels) stack collected over the imaging session. The macro quantified mean pixel intensity for each time point (0-30 min). Images were pseudo-colored using an ImageJ lookup table (Jet) to better display pixel intensity (0-255). Tracer and antibody penetration were also quantified ex vivo in coronal sections, as described previously¹. Each slice was analyzed for mean pixel intensity and the average was computed for all 6 sections taken from one brain. For Aβ plaque quantification, 4× montage coronal images were analyzed using Fiji (61). Images were automatically thresholded (Yen method) on the MeX04 channel and ROIs were generated for each plaque. Plaque burden was calculated from the mean number of plaques per cm² from 3 coronal sections in each mouse. The thresholded MeX04 image was converted into a mask and used to calculate percent MeX04-positive area. A mask was generated for the Aβ antibody fluorescence following the same process and the percent area that was both MeX04- and Aβ antibody-positive over the total MeX04-positive area was considered target engagement. The same coronal sections were used to perform a nearest neighbor analysis of the co-labeled plaques and the closest perivascular space using Fiji and Amira (FEI). The three-dimensional reconstruction was done using Amira (FEI) from 100× confocal z-stacks (0.5 μm step sizes, Leica SP8). To estimate the depth of fluorescence detection, mean pixel intensity for seven regions of interest, each 1-mm deep, from the dorsal convexity to the base of the brain (total 7 mm) were drawn for six coronal sections from an individual mouse using Fiji. Background fluorescence was calculated from all the coronal slices of the 5 min time point and the threshold for signal was placed 2 standard deviations above background. An average for all the 1mm ROIs from each coronal section was calculated for individual mice. Tracer was considered present when MPI from all mice in the group were higher than the threshold. BBB permeability to FITC-dextran was quantified as percent area in six coronal sections for each mouse using a thresholding approach. The threshold was established using Fiji (Otsu) on all coronal sections from the positive control group and computing an average for all slices. The average threshold level was then applied to all the sections from the experimental groups.

Radioisotope Influx. To evaluate solute influx into the brain, radiolabeled tracers ³H-dextran (50 μCi) and ¹⁴C-inulin (0.5 μCi) were injected into the cisterna magna as described above. After 30 min, animals were rapidly decapitated, the skull and dura removed, and the brain harvested. Brain radioactivity was normalized to the total radioactivity detected in a 5 μL aliquot put directly into a scintillation vial immediately before intracisternal injection. All brain tissue was weighed and solubilized in 0.5 mL tissue solubilizer (Solvable, PerkinElmer) overnight. Upon solubilization, 5 mL of scintillation cocktail was added (Ultima Gold, PerkinElmer). The injectate controls were treated in the same way as the tissue samples. All samples were analyzed by liquid scintillation spectrometry using a scintillation counter (LS 6500 Multipurpose Scintillation Counter, Beckman Coulter). The radioactivity (disintegrations per minute: dpm) remaining in the brain after injection (percent of injected dose: %ID) was determined as

$$\frac{{R}_{b}}{{R}_{i}} \times 100 ,$$

where R_{b} is the radioactivity remaining in the brain at the end of the experiment and Rᵢ is the radioactivity in the injectate controls for each experiment. Influx percentage was deduced as % ID. Dextran and inulin are inert, polar molecules that are not actively transported within the CNS, and due to their difference in molecular weight, they are ideal tracers for evaluating the presence of bulk flow.

Statistical Analysis. All statistical testing was performed on GraphPad Prism 7 (GraphPad Software). Tests were chosen based on the data set being analyzed and are reported in the figure legends. All statistical testing was two-tailed and exact P values were calculated at a 0.05 level of significance. All values are expressed as the mean±SEM, unless otherwise stated.

Study approval. All experiments adhered to the laws of the United States, regulations of the Department of Agriculture, and performed according to guidelines from the National

Institutes of Health. Experiments were approved by the University Committee on Animal Resources of the University of Rochester (Protocol No. 2011-023) and an effort was made to minimize the number of animals used.

### Example 2 In Vivo Transcranial Fluorescence Macroscopy Allowed Non-Invasive Brain-Wide Imaging of Glymphatic Flow and Confirms 2-Photon and Ex Vivo Microscopic Findings of Reduced CSF Influx in Awake and Aqp4^{-/-} Mice

Prior studies of CSF-interstitial fluid (ISF) exchange within the glymphatic system have utilized ex vivo conventional fluorescence microscopy and in vivo 2-photon microscopy to image CSF- or ISF-based tracer fluxes (21, 22, 28, 29, 35, 36). While superior for evaluation of brain-wide glymphatic function, including its detailed cellular and molecular organization, ex vivo imaging of brain sections lacks dynamic temporal information, requiring that indirect inferences be made about temporal patterns and rates of glymphatic flow. Conversely, in vivo 2-photon imaging permits real-time determination of rates of CSF tracer appearance in cerebral tissues in individual mice, albeit with a relatively narrow field of view and shallow imaging depth (21, 28, 29).

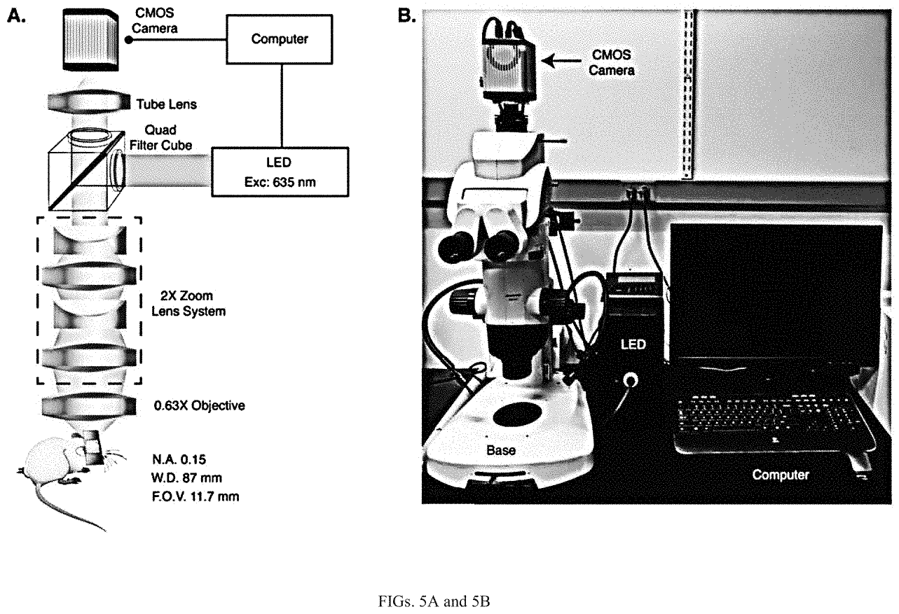

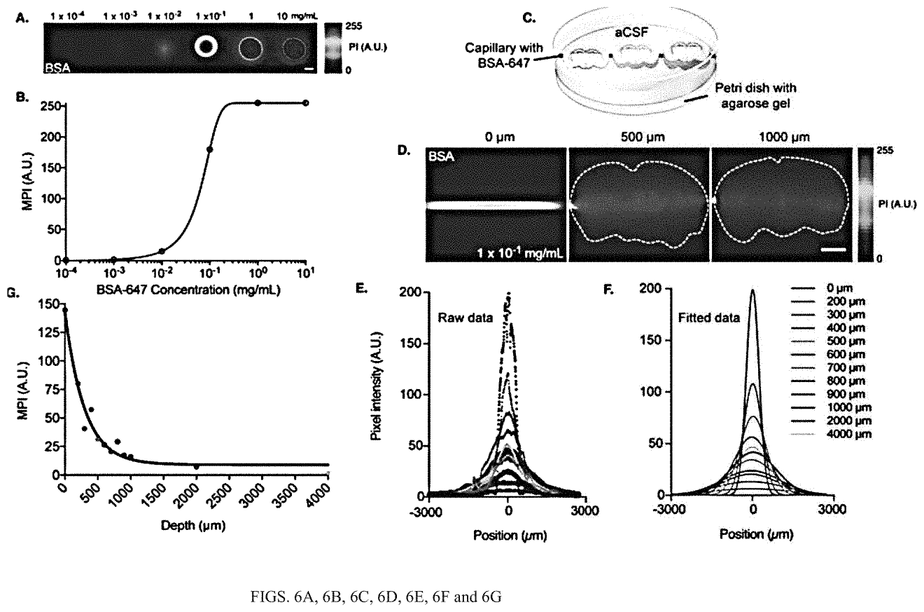

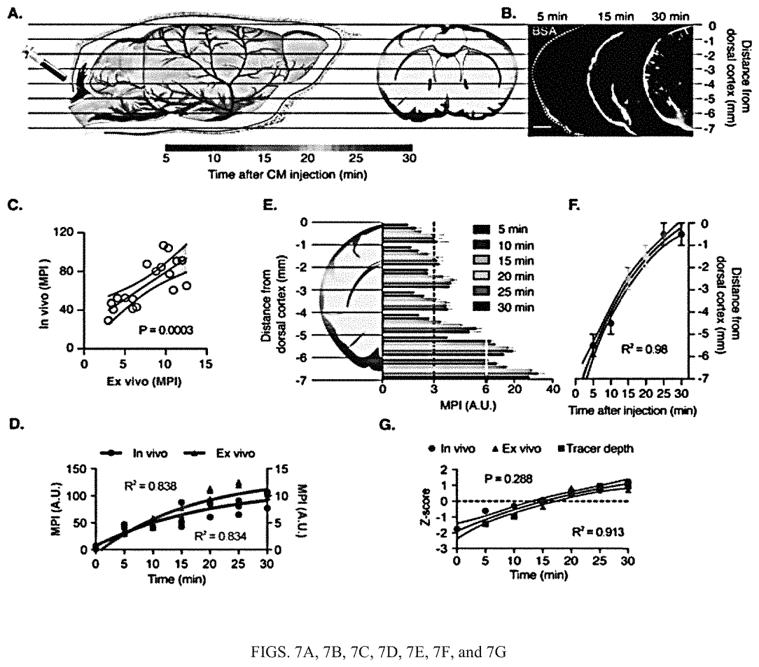

Consequently, inventors developed a new technique for non-invasive in vivo time-lapse imaging of transcranial glymphatic flows using fluorescence macroscopy. (FIG. 5). The technique consists of imaging fluorescent tracers delivered into the cisterna magna of live rodents through an intact skull using an LED illumination source for fluorophore excitation and a macro zoom microscope with high-efficiency CMOS camera for fluorescence detection. The tunable LED can achieve fast-switching between wavelengths and quad filter cubes allow for high-speed, multi-channel image acquisition. The macroscope has a wide field of view enabling mesoscopic imaging of the entire cortical surface and a penetration depth of up to 1-2 mm (FIG. 6).

In validating this technique, inventors first replicated prior in vivo and ex vivo findings of reduced glymphatic CSF influx in awake mice and mice lacking AQP4 (Aqp4^{-/-}) (21, 28). Anesthetized mice in all groups had reflection of the scalp overlying the dorsal calvarium, and cannula placement within the cisterna magna. Groups of wild type and Aqp4^{-/-} mice were subsequently maintained under ketamine/xylazine (KX) anesthesia (KX and KX-Aqp4^{-/-}), while a separate group of wild type mice had a metallic plate secured to the skull for head immobilization, and were then placed in a plastic restraint tube prior to waking up (Awake).

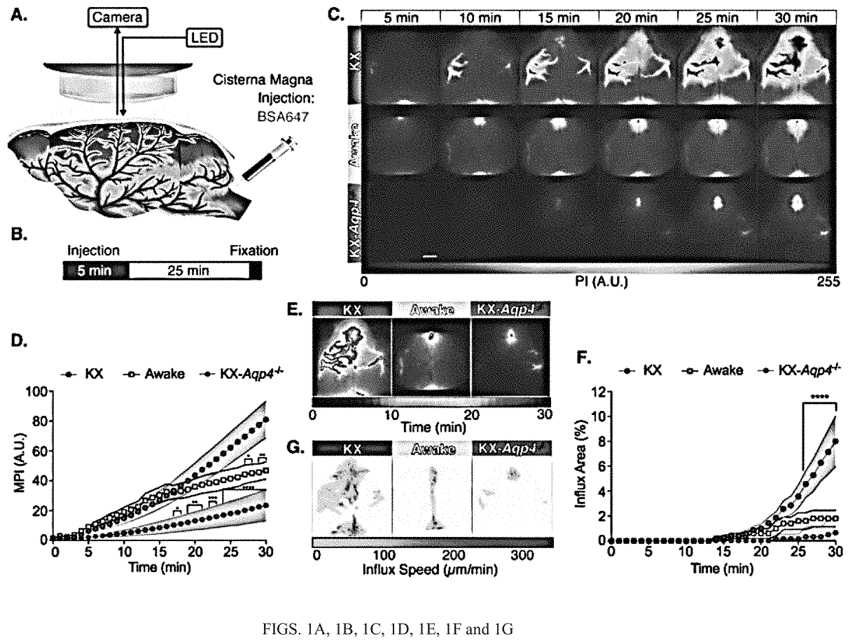

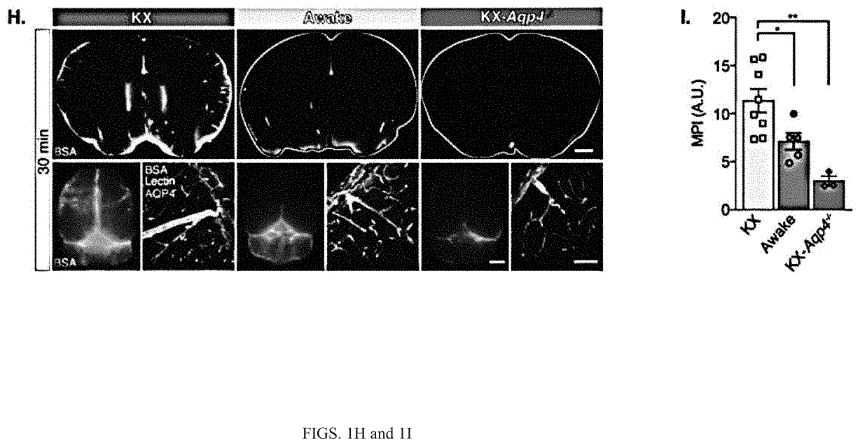

Subsequently, mice were moved to the stage of a fluorescence macroscope for dynamic image acquisition, AlexaFluor647-conjugated bovine serum albumin (BSA-647, 66 kDa) was injected into the cisterna magna (FIGS. 1A and B). Intracisternal infusion caused a mild, transient increase in ICP that normalized before the appearance of tracer (30). Fluorescence was detectable as soon as the tracers arrived in the basal cistern approximately 5-6 mm below the cortical surface (FIG. 7). The influx of BSA-647 into the brain was imaged over 30 minutes before brain harvesting and fixation in 4% paraformaldehyde (PFA) and washed in buffer (FIG. 1B).

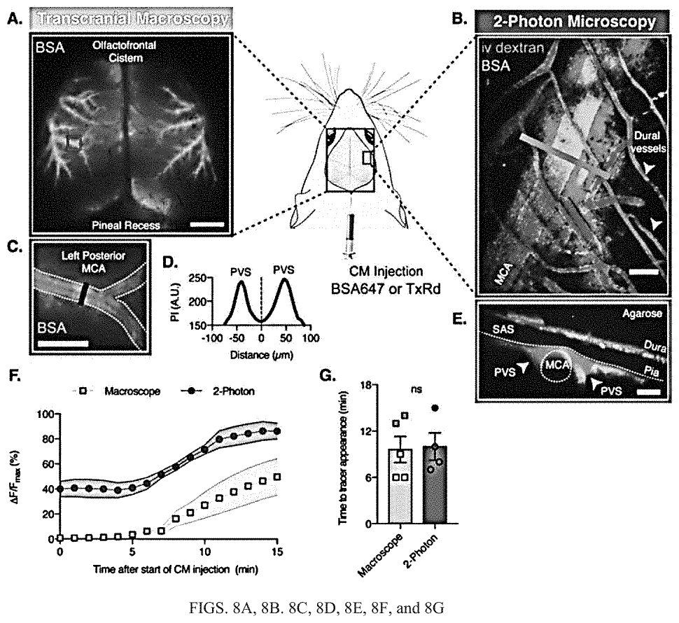

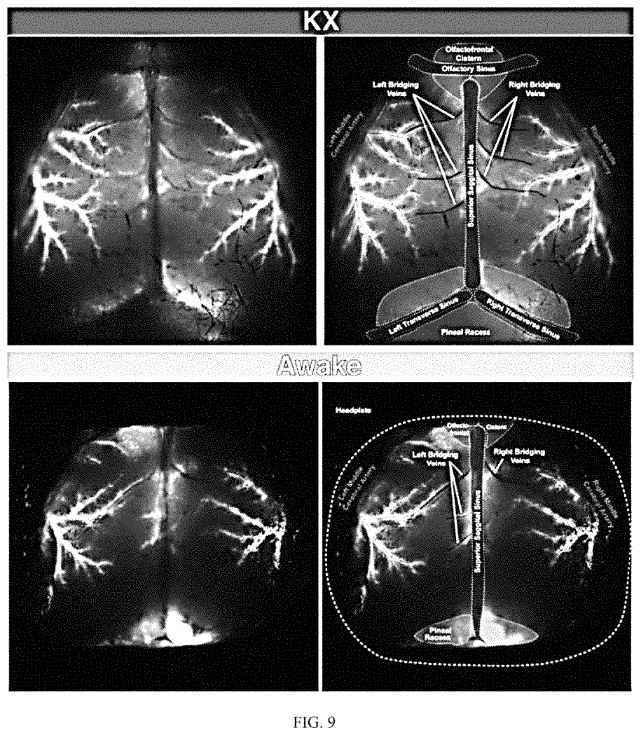

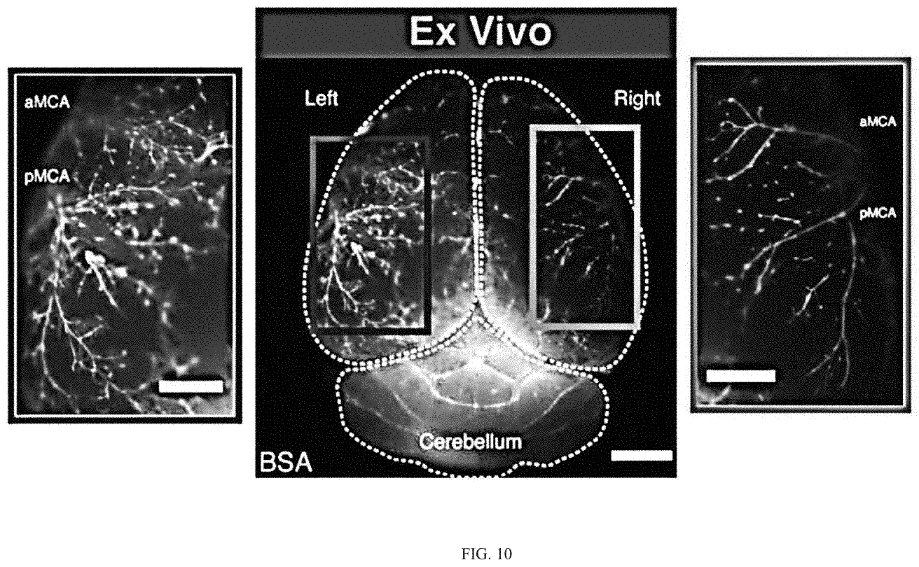

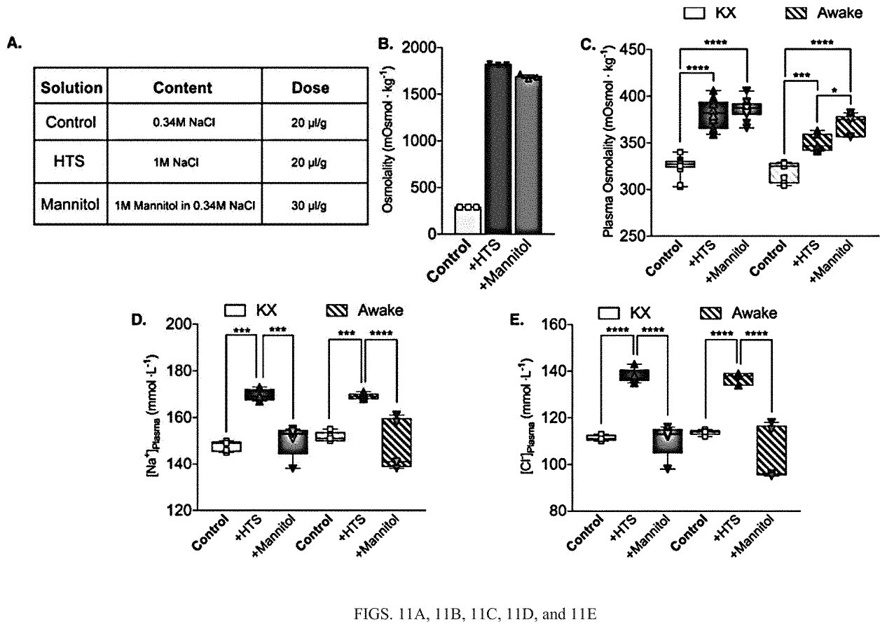

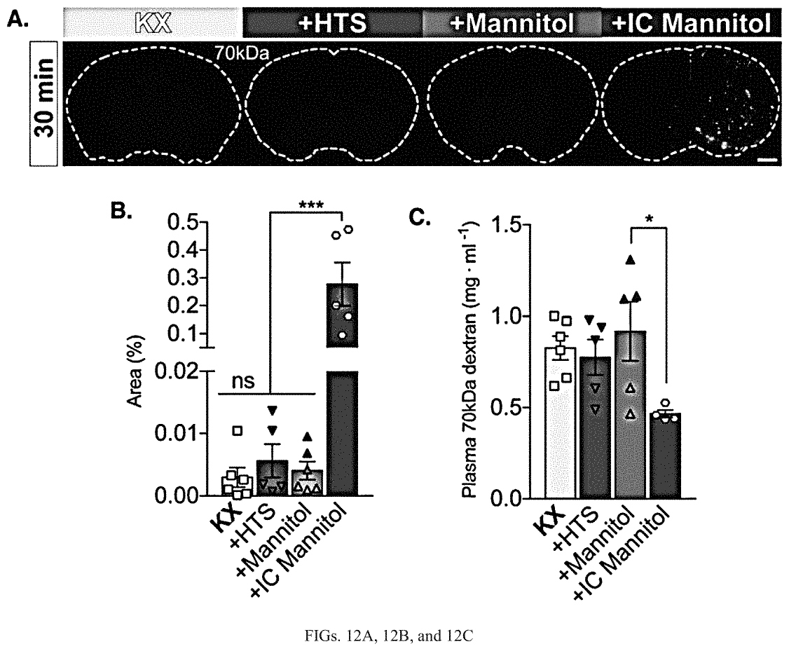

In both anesthetized and awake mice, this transcranial macroscopic approach revealed a pattern of glymphatic CSF influx identical to that previously characterized using in vivo 2-photon (FIG. 8) and ex vivo imaging techniques. The fluorescent protein tracer first appeared within the large rostral and caudal subarachnoid spaces, such as the olfactofrontal cistern and pineal recess, and was carried within minutes over the dorsal cerebral convexity within pial periarterial spaces (FIG. 8); this topography followed the territories of the anterior and posterior cortical segments of the middle cerebral artery (MCA; FIGS. 8 and 9).

It was noted that the infusion pump was stopped at five minutes, or when faint fluorescent signal was first noted at the base of the MCA, indicating that all subsequent tracer appearance could be attributed to physiologic bulk flow. Towards the end of the 30-minute imaging experiment, tracer started to accumulate within the PVS of cortical bridging veins adjacent to the dural sinuses. Confirming previously reported findings, there was significantly less glymphatic influx in both the awake and KX-Aqp4^{-/-} groups compared to the KX mice (FIGS. 1C and D). Interestingly, at the 30-minute time point, glymphatic influx was significantly lower in anesthetized Aqp4^{-/-} mice than in the awake group (FIG. 1D), suggesting that effect on glymphatic function due to deletion of water channels exceeded effects of state of consciousness.

As described above, prior studies of CSF-based delivery of intrathecal solutes to brain could not simultaneously quantify the surface area covered by the tracer and influx kinetics. Even MRI, despite a unique ability to characterize spatial distribution and temporal dynamics, lacks the spatial resolution required to evaluate CSF flows at the level of the PVS (22). Using front-tracking analysis in combination with a macroscopic imaging paradigm to overcome this limitation, we could demonstrate that CSF flow within pial periarterial spaces was higher in KX anesthetized mice and occupied approximately 10% of the dorsal cortical surface compared with under 2% in the awake and KX-Aqp4^{-/-} groups (FIGS. 1E-G). Further, in the awake and knockout groups, perivascular spaces were essentially devoid of tracer, with most of the fluorescent area confined to the olfactofrontal cistern (FIG. 1E).

To exclude the possibility that the imaged glymphatic fluxes were occurring exclusively within the subarachnoid space, the harvested brains underwent conventional fluorescence imaging ex vivo. This analysis showed that the distribution of fluorescent signal throughout the dorsal cortex matched that seen at the terminal 30-minute time point of the sequence in vivo (FIG. 10), thus supporting that the CSF flows observed with the macroscope were occurring at the tissue level and not within the subarachnoid space.

Following coronal sectioning, brain slices were imaged to determine the degree of tracer penetrance into deeper cerebral structures. Again, in parallel to observations during the in vivo imaging series, significantly less tracer was present in brain tissue of the awake and KX-Aqp4^{-/-} mice than in the KX group (FIG. 1H, top panels; 1I). Finally, high resolution confocal images showed that perivascular space tracer distribution was higher in the KX cohort than in the awake and KX-Aqp4^{-/-} mice (FIG. 1H, bottom right panels), confirming the in vivo observation of absent perivascular influx within these groups, and confirming that tracer influx does not occur via diffusion along the pial surface (27).

Collectively, these findings validate the use of fluorescence macroscopy for brain-wide, transcranial imaging of in vivo glymphatic CSF influx, and recapitulate prior work demonstrating the dependence of these flows on perivascular AQP4 expression and the enlargement of interstitial space volume that accompanies sleep (21, 28). Additionally, these new data suggest that increased resistance to fluid flow between the perivascular and interstitial spaces due to AQP4 deletion has a much more profound suppressive effect on glymphatic flow than does wakefulness-related contraction of the interstitial space. The recent critique of the key role of AQP4 in glymphatic function (37) has been challenged by a recent study from four independent labs demonstrating an essential role of AQP4 in solute dispersion in the mouse brain (38).

### Example 3 Plasma Hyperosmolality Significantly Increased CSF Influx in Anesthetized Mice

In this example, assays were carried out to study whether plasma hyperosmolality could enhance the delivery of CSF-based tracers to a greater volume of brain and whether such enhancement occurs via the network of perivascular spaces comprising the glymphatic pathway.

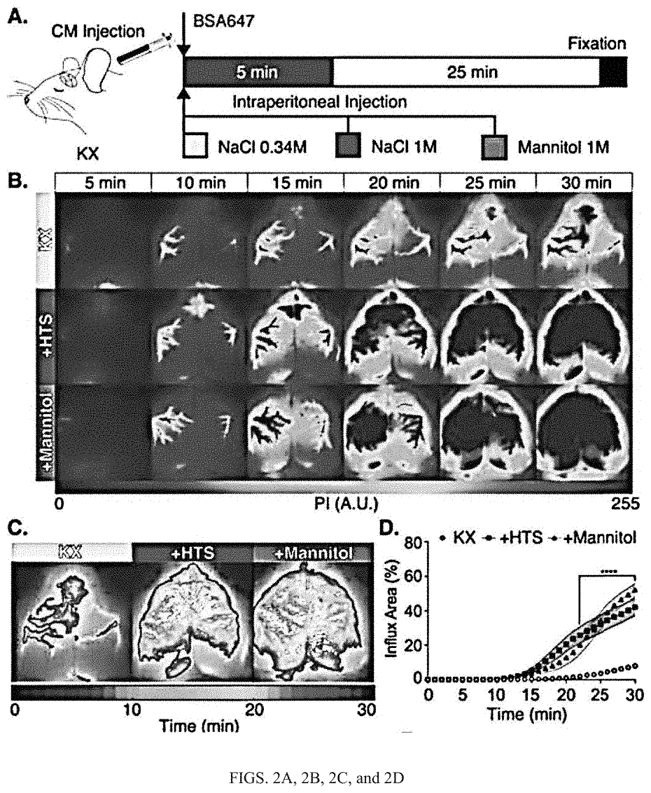

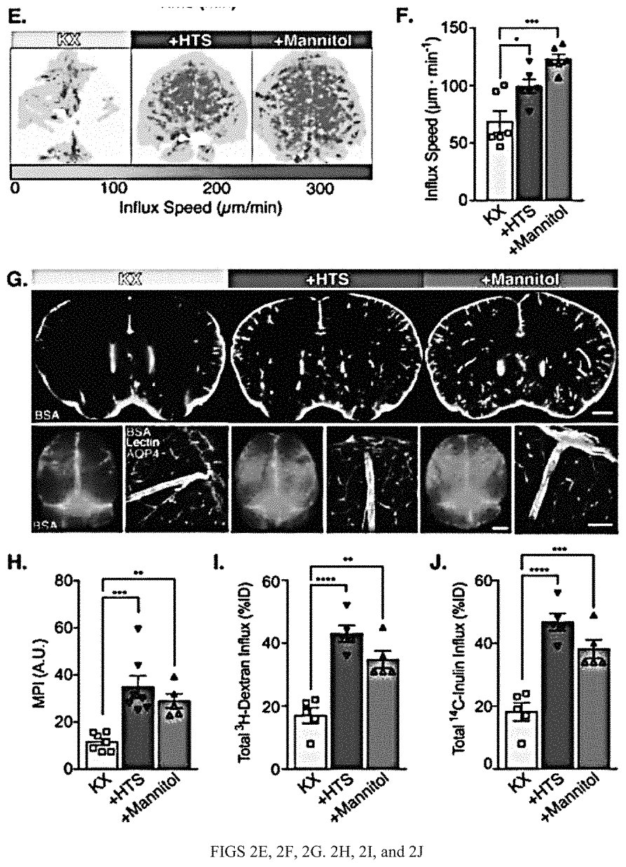

Plasma hypertonicity is often induced clinically for the treatment of elevated ICP using either hypertonic saline (HTS) or mannitol infusions (49). For evaluating effects on glymphatic function, both hypertonicity methods were used to exclude specificity for HTS or mannitol. Briefly, KX-anesthetized wild type mice prepared as above received an intraperitoneal (i.p.) injection of either isotonic saline (KX), hypertonic saline (+HTS), or hypertonic mannitol (+Mannitol) (FIGS. 11A and 11B), consistently resulting in significantly elevated plasma osmolality lasting 30 minutes (FIGS. 11C-E). Notably, at the plasma tonicities achieved in the HTS and mannitol-injected mice, there was no significant increase in BBB permeability (FIGS. 12A-C). In both treatment groups, immediately following intraperitoneal injection of isotonic or hypertonic solutions, BSA-647 was injected to the cisterna magna, and the tracer's area of distribution and kinetics were imaged for 30 minutes using the transcranial microscopic approach described above (FIG. 2A). In agreement with prior studies (27, 50, 51), the main route of CSF tracer entry into brain was via the perivascular spaces surrounding the MCA (FIG. 2B).

These data show that in response to a volume regulatory challenge, the perivascular spaces indeed act as fast conduits to deliver subarachnoid CSF into the volume-depleted brain. However, even more striking was the finding that plasma hypertonicity, due either to

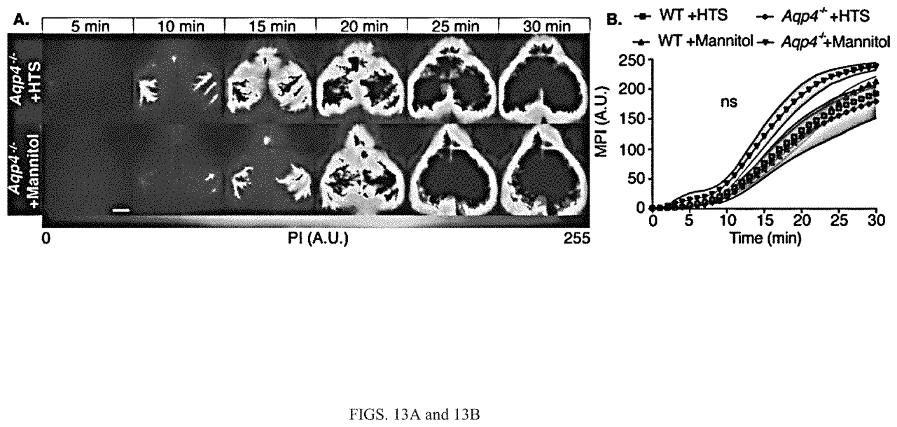

HTS or mannitol challenge, led to a nearly five-fold increase in the influx area, with CSF tracer covering approximately 60% of the dorsal cortical surface, while reducing the time to delivery by roughly half (FIGS. 2C and D). We saw significantly increased influx speeds over the entire cortical surface in the HTS and mannitol groups relative to the isotonic controls (FIGS. 2E and F). This effect was independent of AQP4 expression as plasma hypertonicity was able to override the inhibition of CSF influx seen in Aqp4^{-/-} mice, to levels comparable with wildtypes (FIG. 13).

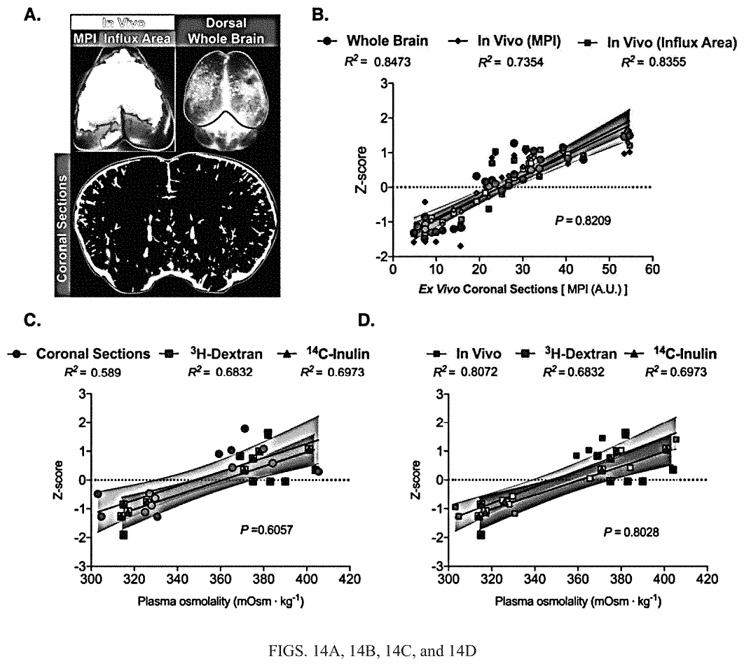

To rule out the possibility that this enhanced tracer influx was limited to the subarachnoid space, the brain and leptomeninges were removed from the calvarium, and cerebral tissues washed prior to conventional ex vivo fluorescence microscopy. Here, the pattern of tracer distribution mirrored that seen at the 30-minute time point of the in vivo imaging experiment, with most of the tracer occupying the subpial or pial perivascular spaces (FIG. 2G, bottom left panels vs 2B, panels under 30 min). Further, imaging of coronal brain sections showed significantly greater tracer signal at locations deep within the brain parenchyma of the HTS and mannitol groups, evidently having been carried into brain via the perivascular spaces of cortical penetrating arteries (FIG. 2G, top and bottom right panels; 2H). Correlation analysis revealed a strong positive relationship between fluorescence signal captured using the newly described in vivo transcranial macroscopic approach, as well as ex vivo whole brain fluorescence, and the traditionally acquired signal from ex vivo coronal sections (FIGS. 14A and B), again suggesting that in vivo tracer dynamics are reflective of tissue level CSF and solute fluxes.

Finally, as the relationship between the amount of fluorescent tracer and relative fluorescence units is linear only at sub-saturated signal levels, we used two radioisotope tracers, ³H-dextran (40 kDa) and ¹⁴C-inulin (6 kDa), to quantify hypertonicity-induced enhancement of glymphatic CSF influx to cerebral tissues. To confirm that entry of tracer within the brain parenchyma was not an artefact of the infusion paradigm, but truly represented physiologic bulk flow, we injected the radioisotope tracers to the cisternal CSF at a rate and volume half that of the fluorescent tracers (1 vs 2 μL/min, respectively over 5 min). We found a roughly 125% increase in the fractional tracer uptake to brain, with approximately 40% of the total injected tracer being delivered to the brain parenchyma in both conditions of plasma hyperosmolality (FIGS. 2I and J). Notably, using plasma osmolality as the predictor, linear regression analysis of tissue radioisotope uptake versus in vivo transcranial or ex vivo coronal section fluorescence area showed significant positive relationships, with similar regression slopes (FIGS. 14C and D). These two independent lines of evidence support the concept that bulk flow mediates tracer influx irrespective of the more than 10-fold difference in molecular weight between fluorescent and radioisotope tracers, and further suggests that non-invasively acquired in vivo fluorescence data and terminal radioisotope studies both offer similarly quantitative assessment of glymphatic dynamics.

### Example 4 Plasma Hyperosmolality Overrides Arousal State-Dependent Inhibition of Glymphatic Function

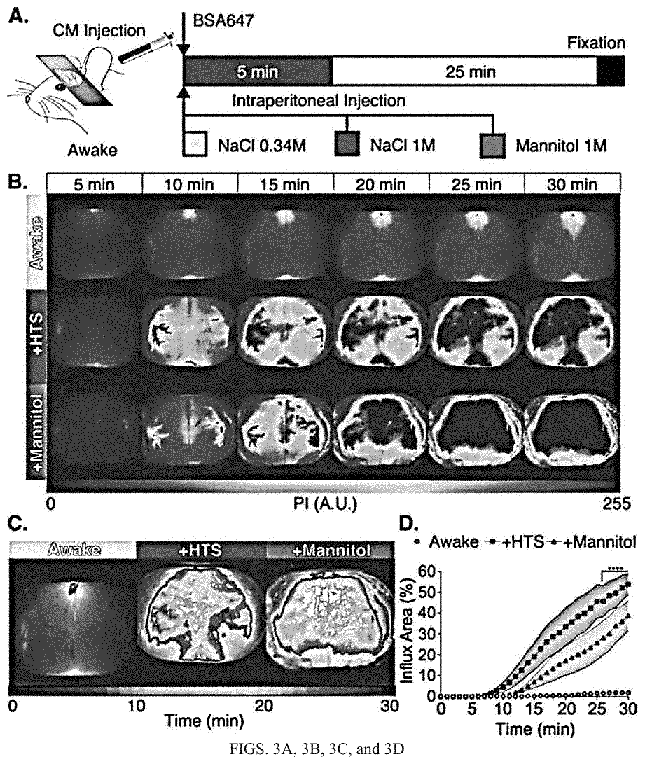

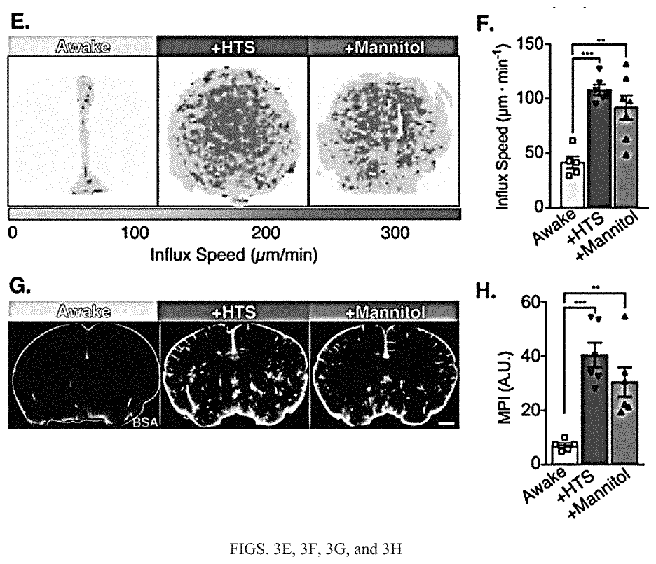

As prior studies have demonstrated profound suppression of glymphatic function in conditions of arousal (28), assays were carried out to examiner whether plasma hypertonicity could overcome wakefulness-related inhibition of CSF influx. Using a similar approach as above, after intraperitoneal injection of either isotonic saline, HTS, or hypertonic mannitol, awake mice received an intracisternal injection of BSA-647 and underwent transcranial macroscopic imaging for a 30-min period prior to brain removal and fixation (FIG. 3A).

Surprisingly, it was found that front-tracking analysis of the in vivo transcranial imaging sequence showed that plasma hyperosmolality evoked a circa 20-fold increase in tracer influx area at 30 min (FIG. 3B-D). Further, tracer influx rates were roughly 1.5-fold faster across the entire dorsal cortex in the awake hyperosmolar challenge groups (FIG. 3E and F). Finally, inspection of coronal sections showed significantly increased tracer influx to deep cerebral structures in both the HTS and mannitol-injected groups, matching observations in the KX-anesthetized cohort. Again, this enhanced tracer influx tended to occur via the perivascular spaces of penetrating arteries (FIGS. 3G and H).

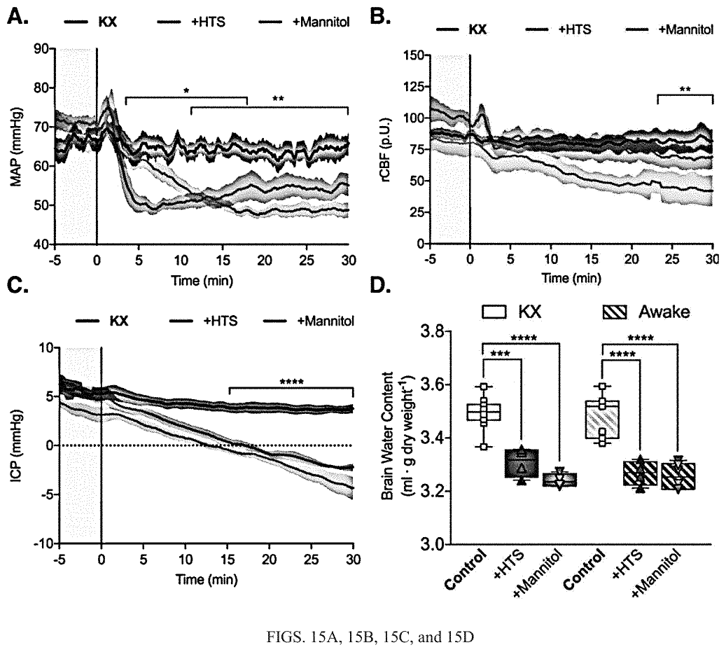

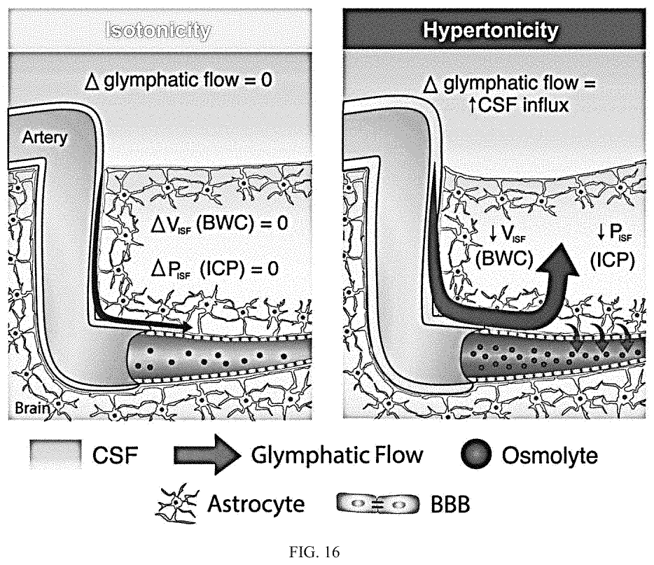

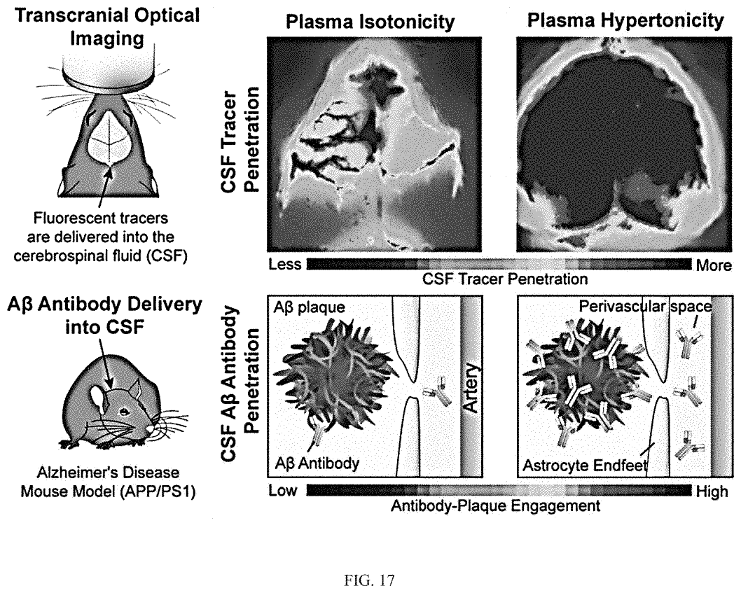

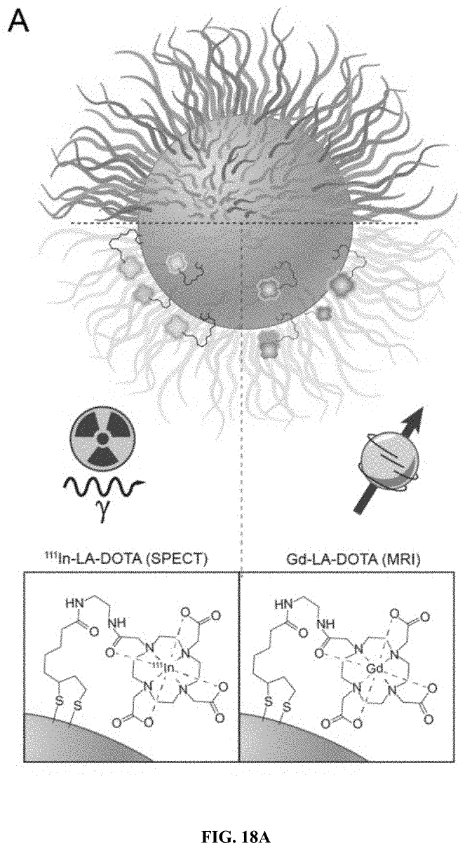

Hyperosmolar agents such as HTS and mannitol have previously been shown to influence mean arterial blood pressure (MAP), cerebral blood flow, and ICP (52, 53). Consequently, assays were carried out to determine if tonicity-induced changes in one of these parameters might be responsible for the observed enhancement of glymphatic CSF influx. Here, it was found that both hyperosmolar challenges reduced MAP, but the effect of HTS was transient, resolving within 15-20 min of intraperitoneal injection (FIG. 15A). Similarly, while there was a slight reduction in relative cerebral blood flow (rCBF) following mannitol administration, rCBF was preserved throughout the duration of the 30-min recording in the HTS group (FIG. 15B). On the contrary, ICP significantly and consistently declined in both hyperosmolar groups relative to the isotonic controls (FIG. 15C). This net negative ICP resulted from the outflow of ISF across the BBB (FIG. 15D), and likely provided the necessary driving force to increase CSF influx to cerebral tissues. Importantly. this transfer of brain water to the vascular column occurred across an intact BBB (FIGS. 12A-C).

### Example 5 Plasma Hyperosmolality Rescues Glymphatic Transport in 6-month-old APP/PS1 Mice and Enhances Delivery and Target Engagement of an Anti-Aβ Antibody

Having demonstrated in conditions of both anesthesia and wakefulness that plasma hyperosmolality increases CSF influx to brain, inventors next sought to determine if this paradigm could be used as a tool to improve brain-wide distribution of experimental therapeutics via the glymphatic pathway. Consequently, it was asked whether plasma hypertonicity could rescue impairment of glymphatic CSF influx in a murine transgenic model of AD (APP/PS1^{+/-}), and further whether an enhancement in glymphatic function could improve brain-wide delivery of an anti-Aβ antibody and its interaction with both perivascular and parenchymal Aβ plaques.

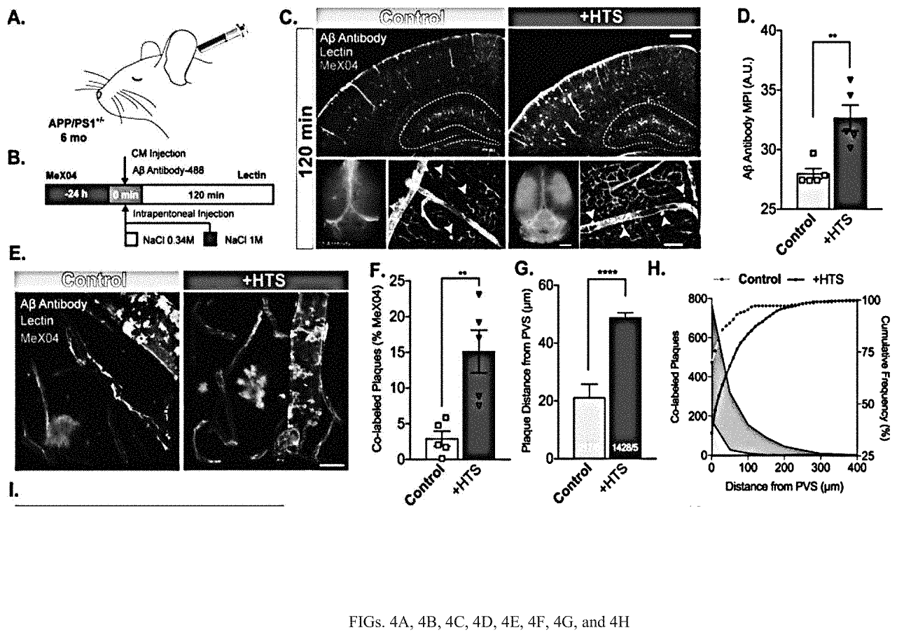

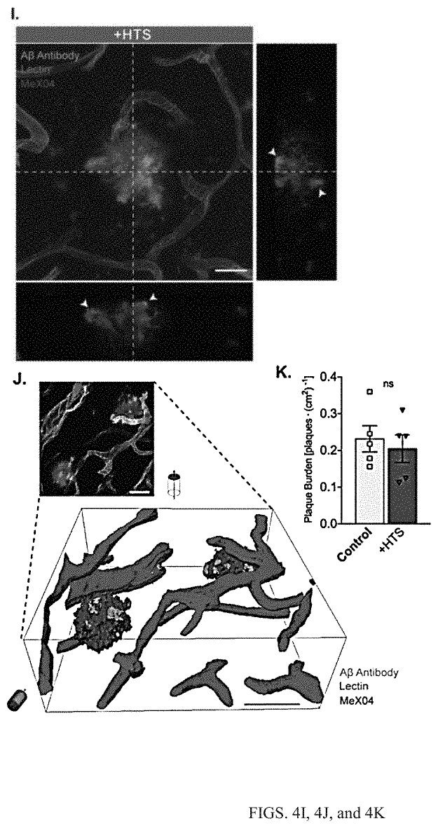

Using an approach similar to that described above, KX anesthetized 6-month old APP/PS1^{+/-} mice received an intraperitoneal injection of either isotonic saline (Control) or hypertonic saline (+HTS) immediately prior to intracisternal delivery of an AlexaFluor488-conjugated anti-Aβ antibody (clone 6E10), which circulated for 120 min prior to brain removal and fixation (FIGS. 4A and B). One day prior to intracisternal antibody injection, intravital labelling of Aβ plaques was obtained with intraperitoneal MeX04 (FIG. 4B). HTS was used to enhance CSF influx due to its lack of effect on rCBF and only transient influence on MAP (FIGS. 15A and B).

It was found that the HTS reversed the glymphatic impairment previously documented in the APP/PS1^{+/-} mice (35) and provoked significantly increased anti-Aβ antibody delivery throughout the cerebrum relative to the isotonic controls. Further, the antibody appeared to gain access to the brain parenchyma via the perivascular spaces (FIGS. 4C and D). While the anti-Aβ antibody was restricted to penetrating arterial perivascular spaces in the control group, there was significant antibody engagement with MeX04⁺ plaques in the HTS-treated mice, suggesting that plasma hypertonicity brings about re-distribution of perivascular solutes to deeper interstitial sites (FIG. 4C, bottom right panels; 4E and F). This is supported by the nearest neighbor analysis showing greater co-labeled plaque distance from the nearest perivascular space in the +HTS group relative to isotonic controls (FIG. 4G).

The greatest abundance of co-labeled plaques occurred in the area immediately surrounding penetrating arteries, with declining frequency at greater distance from the perivascular space. However, nearly all co-labeled plaques occurred within 100 μm of the nearest periarterial space in the control group, whereas this mean separation increased to over 300 μm in the HTS group (FIG. 4H). Three-dimensional reconstructions of confocal z-stacks demonstrated increased antibody binding to plaque surfaces in the +HTS group, although, interestingly, there were no significant differences in plaque burden between groups (FIG. 4I-K). This is likely due to the acute setting of the experiment, being terminated after 120 min of antibody engagement. It is expected that more extended periods of enhanced plaque engagement in the setting of plasma hypertonicity ultimately reduces plaque burden and rescues cognitive performance in AD mice. Present observations also extend the prior finding that genetic deletion of AQP4 in APP/PS1 transgenic mice accelerated cognitive decline and amyloid burden (54).

### Example 6 Material and Methods for Nanoparticle Studies

This example descibes material and methods used in Examples 7-13 bellow.

### Animals

All procedures were approved by the local authorities. A total of 51 male Sprague-Dawley rats (200-300 g, Charles River, Salzburg, Germany) were used. They were housed in groups of four in individually ventilated plastic cages in light- and temperature-controlled rooms. Water and standard laboratory pellets were available ad libitum.

### Materials

All chemicals were used without further purification. All chemicals were purchased from Sigma-Aldrich, except LA-DOTA (2,2′,2″-(10-(2-((2-(5-(1,2-dithiolan-3-yl)pentanamido)ethyl)amino)-2-oxoethyl)-1,4,7,10-tetraazacyclododecane-1,4,7-triyl)triacetic acid) which was purchased from CheMatech (F) and [¹¹¹In]InCl₃ which was bought from Curium Netherlands B.V. (NL). For the synthesis of the complex, ultra trace-select water (Sigma-Aldrich) was used as a solvent, Milli-Q water (Millipore, USA) was used in all other chemical procedures. UV-VIS data were collected on a Shimadzu UV-1800 UV/VIS Scanning Spectrophotometer. AuNPs were characterized by dynamic light scattering (DLS), providing hydrodynamic diameters. The size was measured five times on the same sample and reported as mean square displacement (MSD) calculated number-weighted averages. DLS measurements were carried out in the reaction mixture medium without dilution (sodium citrate and Milli-Q water) at 25° C. DLS measurements were done using a Brookhaven Instruments ZetaPALS apparatus. The size distribution of the nanoparticles was also estimated by analyzing a collection of transmission electron micrographs acquired with an FEI S/TEM 80-300 kV Analytical Titan operating at 300 kV. The nanoparticles suspended in citrate solution (6.5 mM) were drop casted onto a regular copper grid with a holy carbon membrane by using a microliter pipette. Radio-TLC plates (normal phase, eluent=methanol:water 50:50 with 4% ammonium acetate) were analyzed using a Ray test MiniGita apparatus equipped with a Beta detector GMC, or a Perkin Elmer Cyclone® Plus Storage Phosphor System. Radioactivity was quantified with either a CRC®-55tR dose calibrator (activities higher than about 1 MBq) or Hidex 300 SL Automatic TDCR Liquid Scintillation Counter (for activities below about 1 MBq). For the liquid scintillation counting (LSC) measurements a standard curve based on ¹¹¹In was prepared and all samples were within the linear range.

### Synthesis of AuNPs

All glassware was thoroughly rinsed with aqua regia (1:3, 65% HNO₃: 32% HCl) and dried before use. Small AuNPs were prepared according to a modified literature procedure (Piella et al. Chemistry of Materials 28, 1066-1075 (2016)). Freshly prepared aq. trisodium citrate (7.5 mL, 33 mM, 0.25 mmol) was mixed with water (30 mL), aq. K₂CO₃ (250 μL, 150 mM, 38 μmol) and aq. tannic acid (30 μL, 2.5 mM, 75 nmol). The resulting solution was stirred while heated to 70° C. Aq. HAuCl₄ (250 μL, 25 mM, 6.25 μmol) was added, leading to the mixture obtaining a grey color, followed by a gradual change to orange-red. The mixture was stirred at 70° C. for 15 minutes and then allowed to cool to room temperature, furnishing small, citrate-stabilized AuNPs (Au concentration=0.032 mg/mL). The size of the AuNPs was assessed by UV-Vis, showing a maximum absorption at 507 nm, corresponding to a diameter of 4 nm and DLS, giving a number-weighted mean diameter of 4.3±0.4 nm.

For conducting further analyses as well as further use in stability studies (see section 2.5), a part of the citrate-stabilized AuNPs (14.0 mL) was mixed with a freshly prepared aq. solution of methoxy PEG₂₀₀₀ thiol (mPEG₂₀₀₀-SH, 4.0 mg/mL, 200 μL, 800 μg) and stirred at RT for 15 minutes. DLS analysis showed an increase in size to 10.6±2.5 nm indicating successful coating. The PEG₂₀₀₀ coated AuNPs were also analyzed by TEM, giving an average size of 3.6±0.5 nm. For stability studies, 7.0 mL of PEG₂₀₀₀ coated AuNPs were filtered on a 30 kDa cutoff Amicon centrifugation cartridge and resuspended in isotonic HEPES buffer (1000 μL, Au concentration=0.224 mg/mL).

### Synthesis of ¹¹¹In-LA-DOTA

Aq. LA-DOTA (20 μL, 0.5 mM, 10 nmol) was mixed with [¹¹¹In]InCl₃ (in 20 mM aq. HCl, 280 μL, 250 MBq) and aq. sodium acetate (500 μL, 30 mM). The pH of the resulting solution was 6. The solution was heated to 90° C. for 20 minutes to provide the ¹¹¹In-LA-DOTA stock solution. After cooling, 250 μL of the ¹¹¹In-LA-DOTA stock solution was removed and mixed with aq. NaCl (50 μL, 0.53 mM) and an isotonic HEPES buffer (300 μL, 10 mM HEPES, 150 mM NaCl, pH 7.5) to provide the ¹¹¹In-LA-DOTA free complex solution. The activity of this solution was 0.122 MBq/μL.

### ¹¹¹In-Radiolabeling of AuNPs

¹¹¹In-LA-DOTA stock solution (550 μL) was mixed with a dispersion of citrate-stabilized AuNPs (7.0 mL, Au concentration=0.032 mg/mL). The mixture was stirred for 20 minutes at room temperature. The attachment to the AuNPs was monitored by radio-TLC. A freshly prepared aq. solution of methoxy PEG₂₀₀₀ thiol (mPEG₂₀₀₀-SH, 4.0 mg/mL, 100 μL, 400 μg) was then added, followed by stirring at room temperature for 15 minutes. Analysis by UV-VIS spectrophotometry showed a red shift of the maximum absorption to 516 nm, corresponding to a diameter of 9 nm and DLS analysis gave a mean, number-weighted diameter of 12.26±2.30 nm, both indicating successful coating. Analysis by radio-TLC showed that 80% of the radioactivity was associated with the AuNPs. The radiolabeled AuNPs were purified on a 30 kDa cutoff Amicon centrifugation cartridge. The resulting activity associated with the AuNPs was 131.8 MBq while 47.8 MBq were found in the filtrate, corresponding to a radiochemical yield of 73%. Isotonic HEPES buffer (7.0 mL) was added to wash the AuNP-containing filtrand, followed by centrifugation. After this washing step, 128 MBq remained associated with the AuNPs. The radiolabeled AuNPs were resuspended in isotonic HEPES buffer (600 μL) yielding a radioactivity concentration of 0.128 MBq/μL and an Au concentration of 0.373 mg/mL).

### Synthesis of Gd-LA-DOTA

LA-DOTA (100 μL, solution 4.0 mM, 400 nmol) was added to an acid-washed HPLC vial. GdCl₃ (40 μL, solution 10 mM, 400 nmol) was added, followed by ammonium acetate (100 μL, solution 100 mM) and water (760 μL). The pH of the solution was 6.5. The solution was heated to 85° C. for 45 minutes and left for cooling down to room temperature, giving the Gd-LA-DOTA complex solution.

### Gd-Labelling of AuNPs

A dispersion of citrate-stabilized AuNPs (380 mL) with composition and concentration similar to the batch used in the synthesis of the AuNPs (gold concentration=0.032 mg/mL) was prepared. (DLS analysis showed a number weighted NP diameter of 2.7±1.4 nm and UV-visible spectrum, a maximum of the absorption band at 506 nm, corresponding to a size of 3.5 nm). The Gd-LA-DOTA complex solution (whole batch, 1000 μL) was added to the citrate coated AuNPs dispersion. The mixture was stirred at room temperature for 30 minutes. A freshly prepared aq. solution of methoxy PEG₂₀₀₀ thiol (mPEG₂₀₀₀-SH, 4.0 mg/mL, 6.0 mL, 24.0 mg) was then added, followed by stirring at room temperature for 20 minutes (DLS analysis showed a number weighted mean diameter of 10.93±2.70 nm). The Gd-labelled AuNPs were then filtered on a 30 kDa cutoff Amicon centrifugation cartridge, washed with 10 mL of isotonic HEPES buffer, and resuspended in a final total volume of 500 μL (volume adjustment done with addition of isotonic HEPES buffer). The Au and Gd concentrations in the final sample were measured by ICP: Au, 24.65 mg/mL; Gd, 0.058 mg/mL. This corresponds to a Gd-labelling yield of 46.8%.

### Stability of Labelled AuNPs in Brain Homogenate and CSF

To obtain CSF and brain homogenate for assessing AuNP stability, rats were anesthetized with subcutaneous ketamine (100 mg/kg) and dexmedetomidine (0.5 mg/kg) and fixed to a stereotaxic frame after verification of loss of response to painful stimuli. The cisterna magna was exposed and CSF was carefully drawn using a syringe and a 30 G needle. The rats were euthanized, the brains were quickly dissected and cut in four quarters. Each quarter was mixed in 5 mL phosphate-buffered saline (PBS) using a homogenizer. Both the CSF and brain homogenates were stored at —80° C. To allow UV-VIS characterization at low radioactivity level, radiolabeled AuNPs were mixed with non-radiolabeled, PEG-coated AuNPs from the same batch (see section 2.2). The gold concentration was the same in both AuNPs samples (0.373 mg/mL). Plastic size exclusion chromatography (SEC) cartridges (4 cm) were packed with Sephacryl® S300HR, which has been previously used for tissue stability studies on nanoparticles (Frellsen, A. F. et al. Mouse Positron Emission Tomography Study of the Biodistribution of Gold Nanoparticles with Different Surface Coatings Using Embedded Copper-64. ACS Nano 10, 9887-9898 (2016) and Seo, J. W. et al. Liposomal Cu-64 labeling method using bifunctional chelators: poly(ethylene glycol) spacer and chelator effects. Bioconjug Chem 21, 1206-1215 (2010)). PBS was used as eluent. Onto such cartridges, samples of radiolabeled AuNPs or ¹¹¹In-LA-DOTA free complex (both: 100 μL) were applied and separated.

Radioactivity levels in the eluted fractions were monitored, and UV-VIS spectra (200-800 nm) were recorded. Samples were also analyzed by radio-TLC. T=0 samples were analyzed before mixing with tissues.

Free ¹¹¹In-LA-DOTA: The following mixtures were prepared and analyzed: 1) CSF (30 μL)+¹¹¹In-LA-DOTA complex solution (30 μL), 2) Brain homogenate (15 μL)+PBS (15 μL)+¹¹¹In-LA-DOTA complex solution (30 μL). 1% antibiotic (antibiotic antimycotic solution (100×), stabilized with 10,000 units penicillin, 10 mg streptomycin and 25 μg amphotericin B per mL) was added to each vial. The mixtures were incubated at 37° C. and samples were analyzed by radio-TLC at 0, 4, 24, and 48 hours. The experiment was performed in triplicate.

Radiolabeled AuNPs: The following mixtures were prepared and analyzed: 1) Cerebrospinal fluid (175 μL)+AuNP dispersion (175 μL) and 2) Brain homogenate (87 μL)+PBS (88 μL)+AuNP dispersion (175 μL). 1% antibiotic was added to each vial. The mixtures were incubated at 37° C. and samples were analyzed at 4, 24, and 48 hours. The experiment was performed in triplicate.

Biological controls: Neat CSF (50 μL) and brain homogenate (25 μL+25 μL PBS) were analyzed and the UV-VIS spectrum recorded for each fraction, as above. ¹¹¹In-LA-DOTA tissue association control: The following mixtures were prepared: 1) free ¹¹¹In-LA-DOTA complex solution (75 μL)+cerebrospinal fluid (75 μL) and 2) free ¹¹¹In-LA-DOTA complex solution (75 μL)+brain homogenate (37 μL)+PBS (38 μL). After 1.5 hour, 100 μL of each mixture was withdrawn and analyzed by SEC as above. For each fraction collected, the radioactivity as well as the UV-visible spectrum between 200 and 800 nm was measured. For raw data, see Example 13.

### Intracisternal Cannulations and AuNP Infusions

Cisterna magna cannulation was performed as previously described with minor modifications (Xavier et al. J Vis Exp e57378-e57378 (2018)). Rats were anesthetized with a mixture of ketamine (100 mg/kg) and dexmedetomidine (0.5 mg/kg), administered subcutaneously in a volume of 2 ml/kg. After verification of loss of response to toe pinch, animals were placed in a stereotaxic frame and the neck slightly flexed (30°). The atlanto-occipital membrane overlying the cisterna magna was surgically exposed and a 30 G short-bevelled dental needle connected to PE10 tubing was carefully inserted into the intrathecal space. The catheter was fixed to the dura with cyanoacrylate glue and dental cement.

For post-operative analgesia, rats that were imaged for 24 hours received 5 mg/kg carprofen (Rimadyl® vet, 50 mg/mL, Orion Pharma, Espoo, Finland) s.c. at the beginning of surgery. Temperature was monitored and normothermia was maintained with a heating pad. Nanoparticles or the ¹¹¹In-LA-DOTA linker were infused into the cisterna magna with a rate of 1.6 μL/min using a KD Scientific Legato® 130 pump (Holliston, Mass., USA) attached to a Hamilton Gastight 1700 microsyringe (Bonaduz, Switzerland). The total volume of 32 μL was infused over 20 minutes. Five minutes before the AuNP infusion started, rats received either hypertonic saline (1 M, 20 mL/kg i.p.) or isotonic saline (0.154 M, 20 mL/kg i.p.). In the MRI experiments, all rats received a hypertonic saline injection, and the infusion rate of Gd-labelled nanoparticles was the same, but the total infusion volume was 80 μL.

### Single-Photon Emission Tomography (SPECT) and Computerized Tomography (CT)

Radioactivity of the infused dose was measured with VIK-202 dose calibrator (Comecer, Joure, The Netherlands). The Vector4CT (MILabs, Utrecht, Netherlands) system was used for SPECT/CT imaging. SPECT images were acquired with a high energy ultra high resolution rat 1.8 mm pinhole collimator (HE-UHR-RM 1.8 mm ph). Acquired images were reconstructed using Similarity-Regulated Ordered Subsets Estimation Maximization (SROSEM) with a voxel size of 0.6 mm and 5 iterations. Both decays at the 111-In photopeaks (±20%) and background at 20% outside each photopeak window) were individually used for the reconstruction. Two highly accurate energy dependent system matrices for iterative image reconstruction (SROSEM' Vaissier, P. E. B., Beekman, F. J. & Goorden, M. C. Similarity-regulation of OS-EM for accelerated SPECT reconstruction. Physics in Medicine and Biology 61, 4300-4315 (2016)) were used to minimize artifacts in ¹¹¹In imaging with high local uptake and no background activity in surrounding structures. In each matrix the effects of energy dependent pinhole penetration (calculated using a ray tracer, Goorden et al. Physics in Medicine and Biology 61, 3712-3733 (2016)) and intrinsic detector resolution with depth of interaction (DOI) effects in the crystal were modelled. The DOI effect was calculated using GATE Monte Carlo simulations (Jan, S. et al. Physics in Medicine and Biology 49, 4543-4561 (2004)) that was stored in tables and used in the raytracer. Matrices were generated for 171 keV and 245 keV photons and were used during the reconstruction from projections of the corresponding energy peaks. The resulting SPECT images were then added and averaged to obtain the final ¹¹¹In image. Head and full-body CT images were acquired directly after the SPECT scans in the same imaging session. SPECT data were attenuation corrected, decay corrected to the half-life of ¹¹¹In, and corrected for injected activity, with each voxel representing the percentage of the injected dose per cm³ (%ID/cm³).

### SPECT Analysis

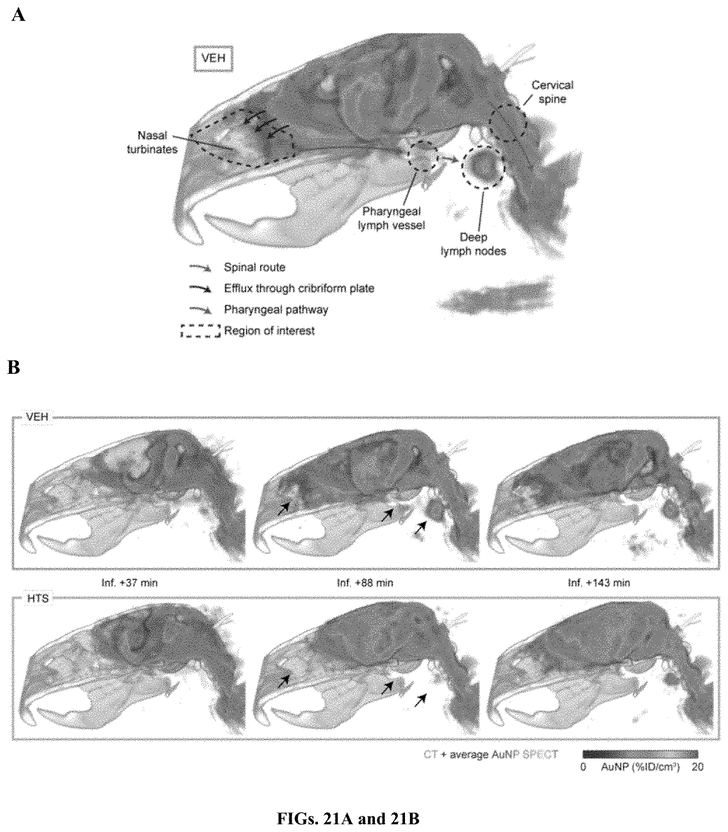

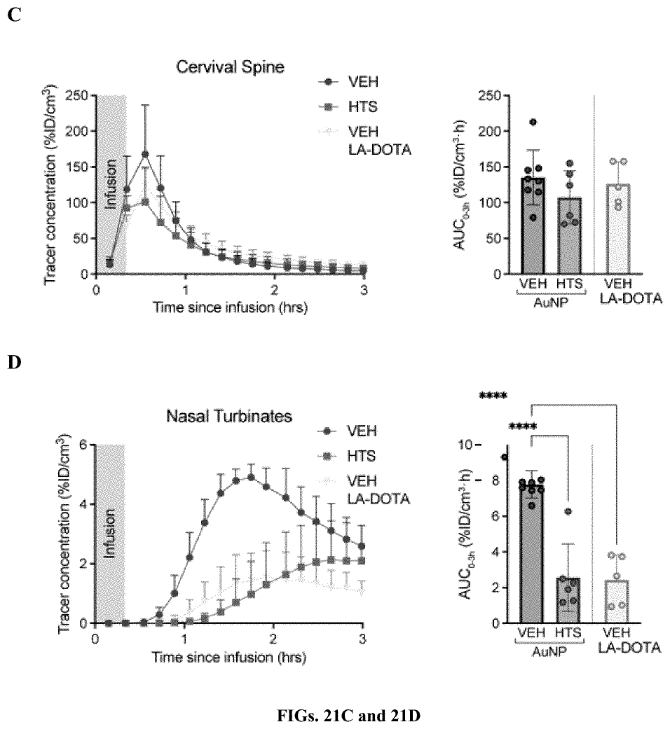

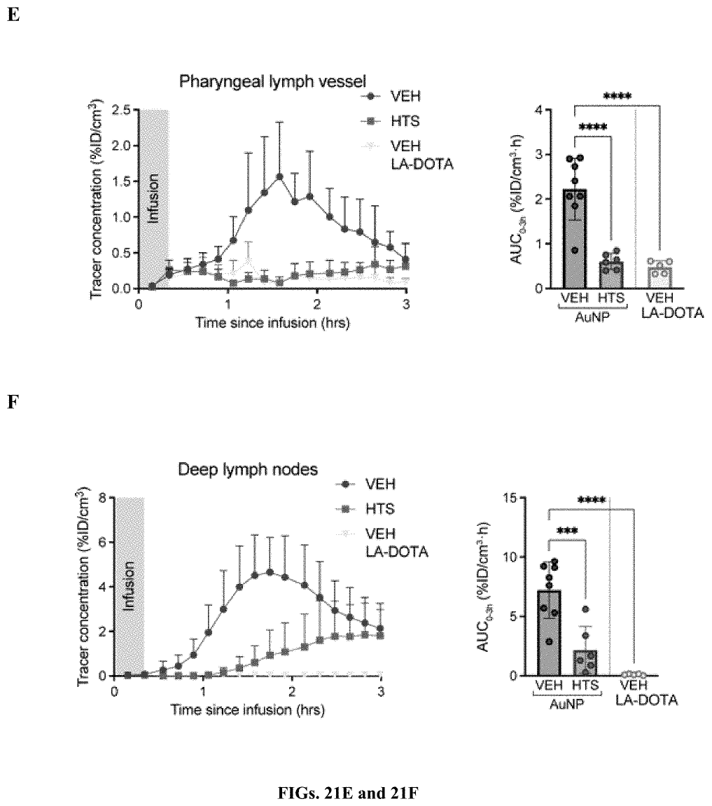

Head and full-body CT images from each imaging session were nonlinearly registered using Advanced Normalization Tools (ANTs) to the appropriate population-based CT template (described below). Regions of interest (ROIs) were then either drawn manually in on individual CT images, manually in the template space, or were computed automatically in the case of the delineation between CSF and brain described below. Manual ROIs were drawn using ITK-SNAP software. Spherical ROIs (Ø 1.4 mm) for striatum and thalamus were placed using the in-house MRI template as reference. Spherical ROIs for nasal turbinates, pharyngeal lymph vessels, and deep cervical lymph nodes were drawn using head CT template as reference and the gross average of all head SPECT images as reference for the deep cervical lymph nodes (FIG. 21A shows an illustration of the placement). Full-body CTs were segmented into the intracranial compartment, spine, kidneys, bladder and lungs, and a spherical ROI (Ø12 mm) was placed in the liver. Time-activity-curves and descriptive statistics were calculated for each ROI using MATLAB 2019B. Soft tissue regions that could not be delineated using in CT images (spherical ROIs) were quantified as percent of the injected dose per cm³ (%ID/cm³), others were quantified in %ID. Group-wise SPECT image time-series were averaged from the SPECT images after registration to template spaces to allow visualization of tracer distribution on average.

### Magnetic Resonance Imaging (MRI)

MRI was carried out on a Bruker BioSpec 94/30 USR magnet interfaced with a Bruker Advance III console controlled by Bruker ParaVision v. 6.0.1 (Bruker BioSpin, Germany). A volume RF-coil (86 mm) was used for transmission along with a 4-channel phased array surface RF receiver coil (Bruker BioSpin, Germany). Dynamic contrast-enhanced MRI (DCE-MRI) consisted of sequential frames of a 3D spoiled gradient echo sequence (FLASH3D, TE: 3.13 ms, TR: 15.8 ms, matrix: 280×173×380, voxel size: 0.1×0.15×0.1 mm, FA: 20). Two frames were acquired prior to contrast agent infusion (20 min), and 18 frames post-infusion (180 min). Rectal temperature and respiratory rate were monitored continuously for the duration of experiment using an MRI-safe monitoring system (SA Instruments, New York, USA).

### MRI Analysis

DCE-MRI sequences were rigidly motion-corrected using ANTs and the brain was extracted by registration to the T2-weighted template. ROIs were manually drawn in the perivascular spaces around the middle cerebral artery and the contrast-enhancement signal was calculated as the percentage change from the pre-contrast baseline (ΔS/S₀).

### Population-Based MRI and CT Templates

To aid and standardize analysis and visualization of SPECT data, inventors used population-based average templates of full-body and head-focused CT as well as of T2-weighted brain MRI. CT templates were created from CT data acquired for this study, while the MRI template was created from MRI from a group of separate rats (12 Sprague-Dawley rats, 7 males, 225-400 g, ketamine/dexmedetomidine anesthesia (100/0.5 mg/kg)) used as control subjects in other studies. Population-based average head CT, body CT and brain MRI templates were created separately using a modified version of the pipeline described by Avants et al. (Avants, B. B. et al. A reproducible evaluation of ANTs similarity metric performance in brain image registration. Neuroimage 54, 2033-2044 (2011)) using ANTs. A representative scan from each included animal was used (full-body CT: n=42, head CT: n=21, brain MRI: n=12). First, a voxel-wise average was calculated of all scans, resulting in the initial template; then, all scans were registered to the initial template and a new voxel-wise average was calculated to produce the next iteration of the template. This process was repeated with increasingly complex registration steps (rigid, affine, and nonlinear) repeating each step until there was little change from between iterations. Full body CT images were thresholded at 500 Hounsfield units (HU) before the template building process. T2-weighted MRI was acquired using the same setup as the dynamic contrast-enhanced MRI; imaging consisted of T2-weighted TurboRARE (TE: 24.1 ms, TR: 16 s, Echo spacing: 8.033, RARE factor: 8, matrix: 375×250, FOV 30×20 mm, in-plane resolution: 0.08×0.08 mm, 128 slices, 220 μm slice thickness, 110 μm overlap, 8 repetitions). Eight serial images from each animal were rigidly motion corrected, averaged, and bias-field corrected using the N4 software (Tustison, N.J. et al. N4ITK: improved N3 bias correction. IEEE Trans Med Imaging 29, 1310-1320 (2010)). Affine registrations between the three templates were calculated semi-automatically using ITK-SNAP software v. 3.8.0. The intracranial space was manually segmented using ITK-SNAP and tissue segmentation of the intracranial space into brain tissue and CSF was computed from the MRI template using ANTs Atropos (Avants, B. B., Tustison, N.J., Wu, J., Cook, P. A. & Gee, J. C. An open source multivariate framework for n-tissue segmentation with evaluation on public data. Neuroinformatics 9, 381-400 (2011)) based on an initial manual threshold.

### Statistics and Software

Image preprocessing was carried out using Python 3.8.5 and image registration, bias field-correction, and automated image segmentation were performed with Advanced Normalization tools (ANTs). Extraction of time-activity-curves and derived statistics was carried out using MATLB 2019B. Unless otherwise noted, statistical comparison consisted of unpaired t-test in the case of two groups and ordinary one-way ANOVA with Dunnett's multiple comparisons test in the case of several groups. Statistical tests were carried out using GraphPad Prism 9.2.0.

### Example 7 Synthesis and Characterization of Small Gold Nanoparticles

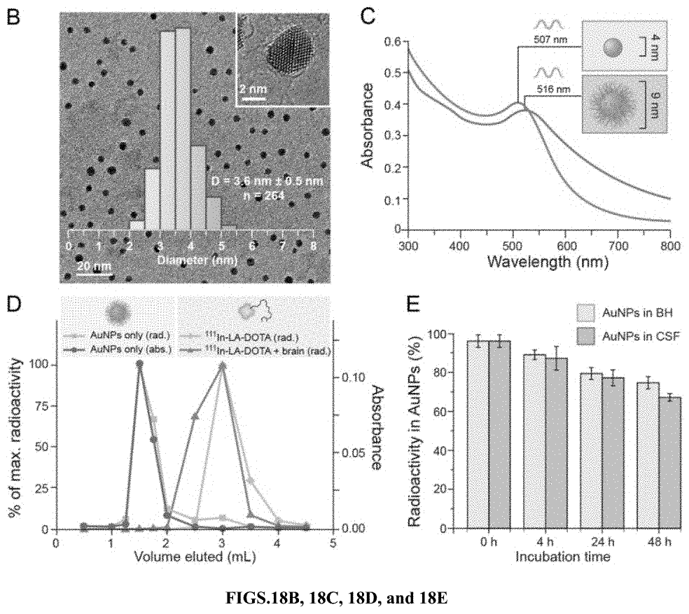

Small AuNPs coated with polyethylene glycol (PEG₂₀₀₀) and labelled with either ¹¹¹In or Gd were prepared for imaging by SPECT or MRI, respectively (FIG. 18A). Citrate-stabilized, uncoated AuNPs were first formed by reduction of HAuCl₃ solutions, which was followed by labelling and polyethylene glycol (PEG) coating (FIG. 18A, top). Labelling was achieved via complexation of ¹¹¹In³⁺ or Gd³⁺ with a conjugate of lipoic acid (LA) and the macrocyclic chelator 1,4,7,10-tetraazacyclododecane-1,4,7,10-tetraacetic acid (DOTA). DOTA is a well-established chelator, providing stable complexes for both In³⁺ and Gd³⁺ (Sartori, A. et al. Synthesis and preclinical evaluation of a novel, selective In-111-labelled aminoproline-RGD-peptide for non-invasive melanoma tumor imaging. Medchemcomm 6, 2175-2183 (2015) and Sherry, A. D., Caravan, P. & Lenkinski, R. E. Primer on Gadolinium Chemistry. Journal of Magnetic Resonance Imaging 30, 1240-1248 (2009)). The chelates were synthesized first, after which they were attached to the AuNPs via interaction of the disulphide moiety with the gold surface (FIG. 18A, bottom). This was followed by saturation of the AuNP surface with methoxy-PEG₂₀₀₀ thiol, to provide the final ¹¹¹In—AuNPs or Gd—AuNPs for in vivo use.

The prepared AuNPs were characterized by dynamic light scattering (DLS), showing hydrodynamic diameters of 4.3±0.9 nm for the citrate-stabilized, uncoated AuNPs before PEG coating and 12.3±2.3 nm (¹¹¹In) or 10.9±2.7 nm (Gd) after labelling and PEG coating. Transmission electron microscopy showed gold core diameters of 3.6±0.5 nm (n=264) (FIG. 18B). As a further measure of size, both uncoated and PEG-coated AuNPs were analysed by UV-VIS spectroscopy, with absorption at 507 nm for uncoated AuNPs and 516 nm for PEG coated AuNPs, corresponding to 4 nm and 9 nm, respectively (Piella et al. Chemistry of Materials 28, 1066-1075 (2016)) (FIG. 18C).

The stability of the labelled AuNPs was first studied in vitro in CSF and rat brain homogenate (BH), both media being highly relevant for the investigated delivery route. To enable facile quantification by radioactivity measurements, the ¹¹¹In-labeled AuNPs were used. Incubated mixtures were monitored for detachment of the label from the AuNP surface for 48 hours, via separation of AuNPs and the detached small-molecular label by size exclusion chromatography (SEC) on a Sephacryl S300HR resin.

For each eluted fraction, inventors measured radioactivity and absorption at 515 nm (AuNP local maximum) (FIG. 18D, Example 13). Approximately 95% of the radioactivity was associated with the AuNPs at t=0. After 4 hours, 13% (CSF) and 11% (BH) of ¹¹¹In-LA-DOTA had been detached from the AuNPs, dropping slightly to 23% (CSF) and 20% (BH) after 24 hours, and 33% (CSF) and 25% (BH) after 48 hours (FIG. 18E, Example 13). As controls, free ¹¹¹In-LA-DOTA was incubated with both CSF and BH at 37° C., analysed in the same way, and additionally monitored by radio thin layer chromatography (radio-TLC, Example 12). This demonstrated that free ¹¹¹In-LA-DOTA does not bind to any biological material eluting in the large molecular fraction together with the AuNPs, which could have provided false positive results (FIG. 18D). Inventors observed no indications of release of ¹¹¹In from the chelator over 48 hours, suggesting that the ¹¹¹In-LA-DOTA complex is stable in the employed biological media, as is also widely reported (Sartori, A. et al. Medchemcomm 6, 2175-2183 (2015); Laznickova et al. Journal of Radioanalytical and Nuclear Chemistry 273, 583-586 (2007), and Carlucci, G. et al. Mol. Pharmaceutics 10, 1716-1724 (2013)). This supported that the observed release stemmed from detachment of the LA-DOTA complex from the AuNP surface, making the data applicable to both the ¹¹¹In- and Gd-labelled variants.

### Example 8 Systemic Hypertonic Saline Enhances Brain-Wide Distribution of Small Gold Nanoparticles

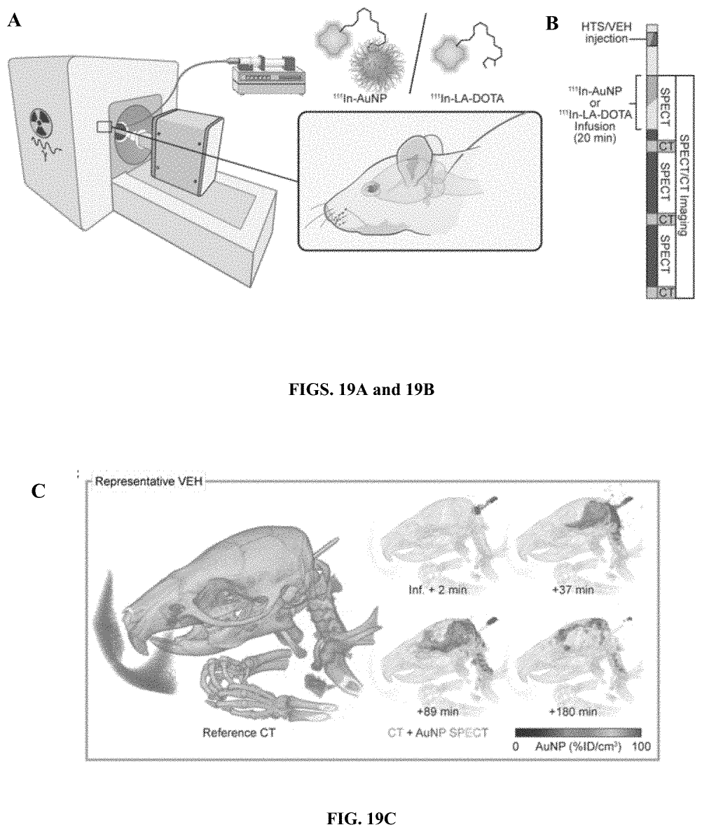

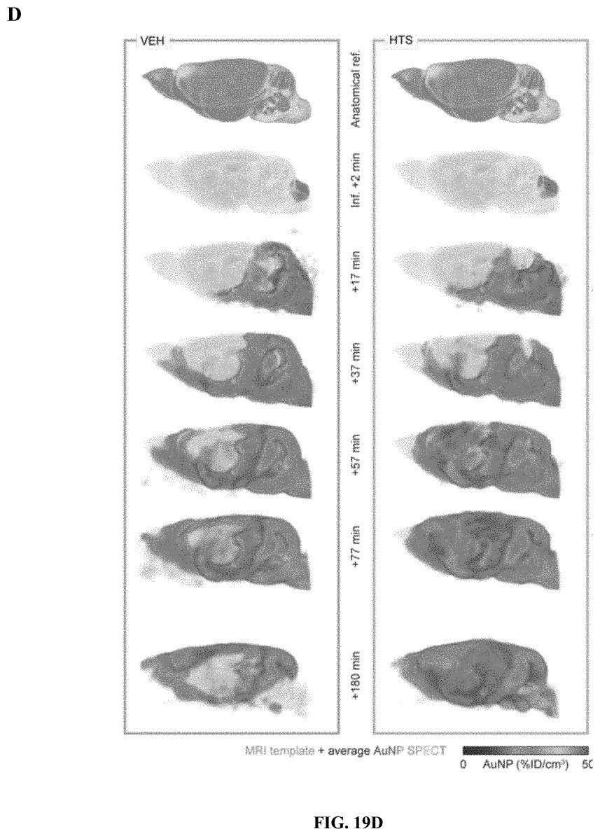

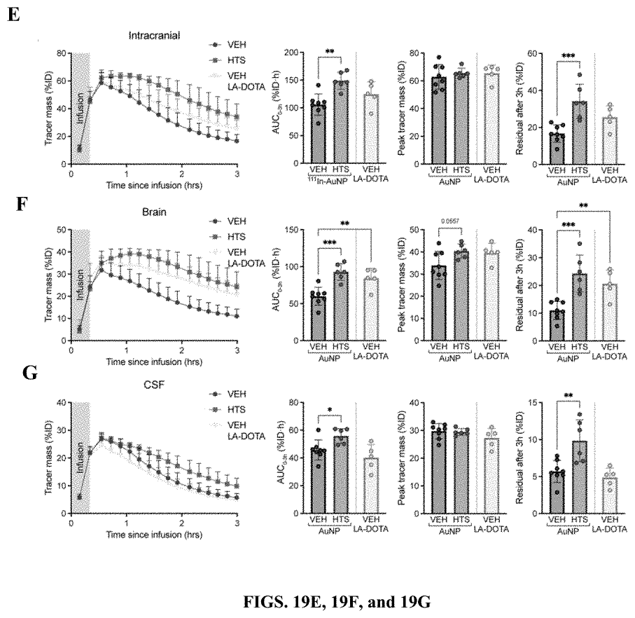

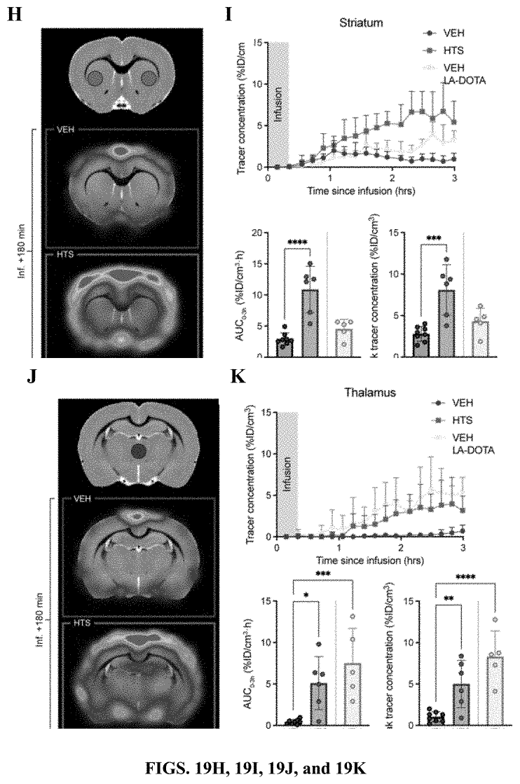

SPECT imaging was used to dynamically follow the distribution of ¹¹¹In-LA-DOTA labelled AuNPs (¹¹¹In—AuNPs) infused to the CSF-filled cisterna magna (FIG. 19A). Five minutes prior to AuNP infusion (32 μl, 2.2±0.7 MBq, 1.6 μl/min), anaesthetized Sprague-Dawley rats received a slow intraperitoneal injection of either hypertonic (HTS, 1 M, 20 ml/kg, 40 mOsm/kg) or isotonic saline (vehicle; VEH, 0.154 M, 20 ml/kg) (FIG. 19B). To enable visualization and quantification at the group level, head-focused SPECT and computed tomography (CT) images were registered into a common coordinate system using an MRI-derived template as reference.

In both groups, the ¹¹¹In—AuNP dispersion flowed from cisterna magna through the subarachnoid space along surface arteries to the circle of Willis and further along the posterior, middle and anterior cerebral arteries as previously reported (Iliff, J. J. et al. Brain-wide pathway for waste clearance captured by contrast-enhanced MRI. J. Clin. Invest. 123, 1299-1309 (2013)) (FIG. 19C). While ¹¹¹In—AuNP availability as a percentage of injected dose (%ID) peaked at comparable amounts (VEH: 62.8±8.9, HTS: 65.6±3.4%ID, p=0.696), the residual ¹¹¹In—AuNP mass 180 minutes after infusion start was doubled with HTS treatment (VEH: 16.7±4.7, HTS: 34.1±9.3%ID, p=0.0004) and HTS significantly increased the overall intracranial ¹¹¹In—AuNP exposure during the first three hours (AUC₀₋₃ₕ) by 40% (VEH: 105.7±19.2, HTS: 148.7±15.3%IDh, p=0.0014) ((FIG. 19D).

In addition to validating the stability of the ¹¹¹In-LA-DOTA-AuNP complex in vitro (FIG. 18E), inventors compared the in vivo distribution of the ¹¹¹In-LA-DOTA linker and the full ¹¹¹In-LA-DOTA-AuNP complex in rats that received intraperitoneal vehicle. While the overall intracranial AUC₀₋₃ₕ of the ¹¹¹In—AuNPs and the small-molecular linker did not differ (FIG. 19E), the linker showed higher penetration to the brain (FIG. 19F) and to the thalamus (FIG. 19K), suggesting size-dependent brain penetration of tracers as has been previously demonstrated (Iliff, J. J. et al. A paravascular pathway facilitates CSF flow through the brain parenchyma and the clearance of interstitial solutes, including amyloid β. Sci Transl Med 4, 147ra111 (2012)).

Using an MRI-derived template derived from a separate group of rats of the same strain and age, the intracranial compartments were roughly divided into brain parenchyma and CSF to approximate which compartment caused the increase in intracranial ¹¹¹In—AuNP exposure. Due to the small size of the subarachnoid and perivascular spaces and relatively low imaging resolution of SPECT, there was significant signal spill-over between the brain parenchyma and CSF in the intracranial compartment. From the time-activity-curves for CSF and brain (FIG. 19E-F), the increased ¹¹¹In—AuNP exposure appeared largely in the brain parenchyma. To test this, inventors compared the ratios of brain exposure to the intracranial exposure (AUC_{0-3h brain}: AUC_{0-3h intracranial}) between groups and found a higher ratio in the HTS compared with the isotonic vehicle group (VEH: 0.565±0.018, HTS: 0.624±0.021, p=0.0001, t-test), implying enhanced delivery of ¹¹¹In—AuNPs to the brain by HTS.

To further examine the penetration of ¹¹¹In—AuNPs to the brain parenchyma, inventors measured activity in deep brain regions, unaffected by spill-over activity from CSF. Using the MRI template as an anatomical guide, inventors placed small spherical regions of interest (Ø1.4 mm) centrally in thalamus and in the bilateral caudate putamen (FIG. 19H-K) to measure ¹¹¹In—AuNP concentrations. HTS increased the exposure (AUC₀₋₃ₕ) to ¹¹¹In—AuNPs 3.7- and 12-fold in the striatum and thalamus, respectively, demonstrating dramatically enhanced deep brain penetration with HTS.

### Example 9 Brain-Wide Periarterial Distribution of Small Gold Nanoparticles Imaged with Dynamic Contrast-Enhanced Magnetic Resonance Imaging

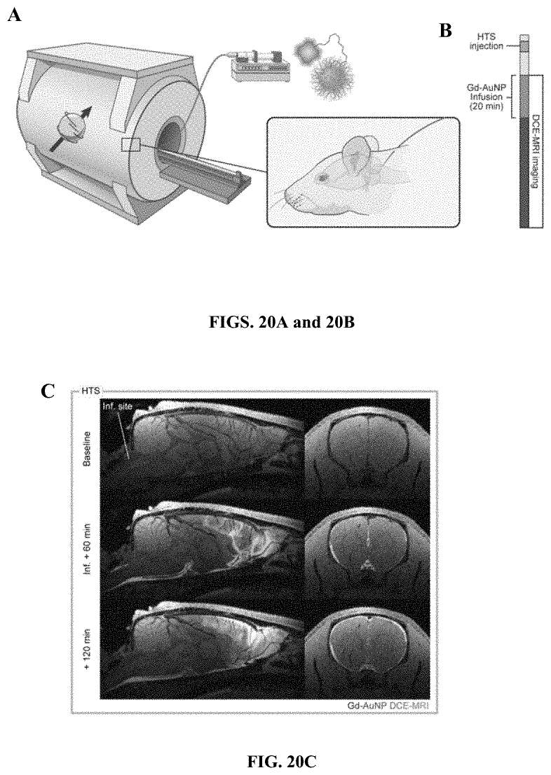

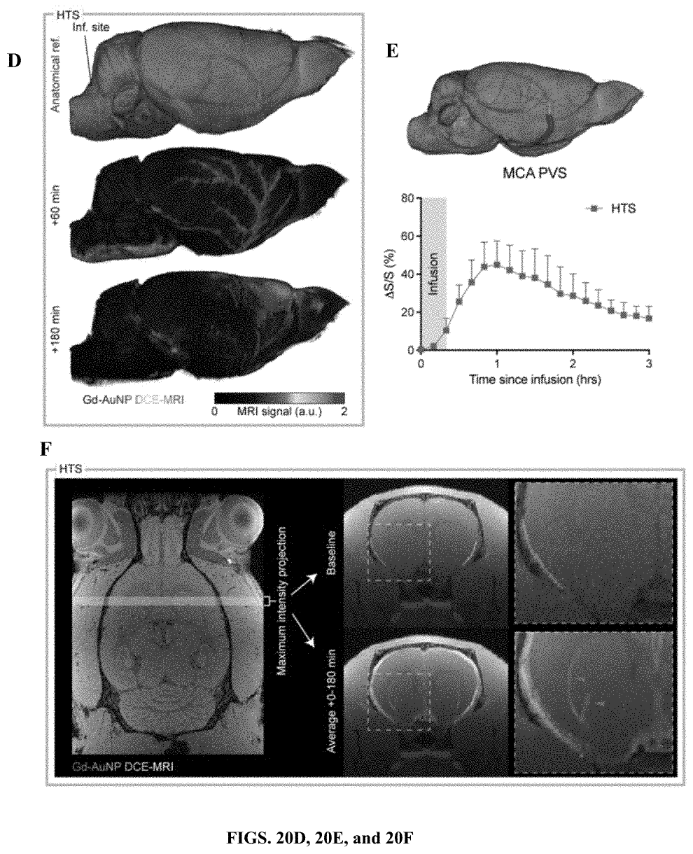

Since SPECT imaging has a limited resolution to visualize the exact delivery routes to the brain, dynamic contrast-enhanced MRI (DCE-MRI) was then performed using gadolinium-labelled AuNPs (Gd—AuNPs) as contrast agent (FIG. 20A). To compensate for the decreased sensitivity of MRI as compared with SPECT, the infused volume of Gd—AuNPs was increased to 80 μL while maintaining the same infusion rate that has been shown not to influence intracranial pressure even during long infusion times (Iliff, J. J. et al. Brain-wide pathway for waste clearance captured by contrast-enhanced MRI. J. Clin. Invest. 123, 1299-1309 (2013) and Yang, L. et al. Evaluating glymphatic pathway function utilizing clinically relevant intrathecal infusion of CSF tracer. J Transl Med 11, 107 (2013). The infusion was initiated 5 minutes after the administration of systemic HTS (FIG. 20B). Dynamic MRI imaging affirmed the brain-wide distribution of Gd—AuNPs in the perivascular spaces of arteries in pial arteries (FIG. 20C-D). Periarterial Gd—AuNP concentration measured in the perivascular space of the left middle cerebral artery showed a marked peak followed by gradual decrease (FIG. 20E), concentration peaked at 78±32 minutes after infusion and decreased to 39±17% of the peak after 180 minutes. Importantly, Gd—AuNPs were clearly detected in the periarterial spaces of penetrating arteries (FIG. 20F). Inventors noticed a decrease in background MRI signal following the HTS injection, which was ascribed to a general decrease in brain water content (Plog, B. A. et al. JCI Insight 3, 1188 (2018)). As a result, inventors were not able to quantify the MRI signal increase in the brain parenchyma caused by the presence of Gd—AuNPs.

### Example 10 Hypertonic Saline Decreases the Egress of Intrathecally Administered Small Gold Nanoparticles to Lymphatic Structures

Next, inventors measured ¹¹¹In—AuNP transport along CSF egress routes (FIG. 21A-B). In line with enhanced deep brain uptake, the egress of ¹¹¹In—AuNPs through the nasal turbinates (Proulx Cell Mol Life Sci 78, 2429-2457 (2021)) (FIG. 21D), the pharyngeal lymphatic pathway (Stanton et al. Magn Reson Med 85, 3326-3342 (2021) and Bradbury et al. J Physiol. (Lond.) 339, 519-534 (1983)) (FIG. 21E) and deep cervical lymph nodes (FIG. 21F) were significantly reduced by HTS. The HTS treatment did not significantly change peak concentration or exposure in the cervical spine (FIG. 21C), indicating that HTS mainly reduces shunting of ¹¹¹In—AuNPs from the CSF to lymphatic structures without significant effects on their caudal-directed flow.

The ¹¹¹In—AuNP concentration in both the VEH and HTS group peaked approximately simultaneously in the nasal turbinates and cervical lymph nodes. While the nasal route is an important route of CSF egress (Proulx. Cell Mol Life Sci 78, 2429-2457 (2021)), the fact that ¹¹¹In—AuNPs arrived simultaneously at the nasal turbinates and cervical lymph nodes may indicate that there are fast and more direct egress routes from the intracranial compartment to the cervical lymph nodes than through nasal turbinates. One potential direct route to deep cervical lymph nodes could include egress through the meningeal lymphatic vessels on the ventral side of the brain (Ahn et al. Nature 572, 62-66 (2019)) or via cranial nerve sheaths, as visualized in vivo by Stanton et al. (Magn Reson Med 85, 3326-3342 (2021)).

The kinetics of the free ¹¹¹In-LA-DOTA linker in the efflux pathways were also assessed. Although the intracranial AUC₀₋₃ₕ between the linker and ¹¹¹In—AuNPs did not differ (FIG. 19E), exposure to ¹¹¹In—AuNPs in the nasal turbinates was significantly higher (FIG. 21D), possibly suggesting a more important role of the nasal outflow routes for large molecules. Importantly, the ¹¹¹In-LA-DOTA linker was virtually undetectable in deep lymph nodes (FIG. 21F), suggesting that exposure of the lymphatic structures of the head and neck after intrathecal administration of small-molecular agents is minor and that targeting these lymphatic structures could be facilitated using nanoparticles as drug carriers. The dramatically different kinetics of ¹¹¹In-LA-DOTA-AuNPs and free ¹¹¹In-LA-DOTA linker in the efflux pathways support our in vitro findings that the ¹¹¹In-LA-DOTA-AuNP complex is stable also in vivo.

### Example 11 Small Gold Nanoparticles Show Rapid Overall Elimination from the Body

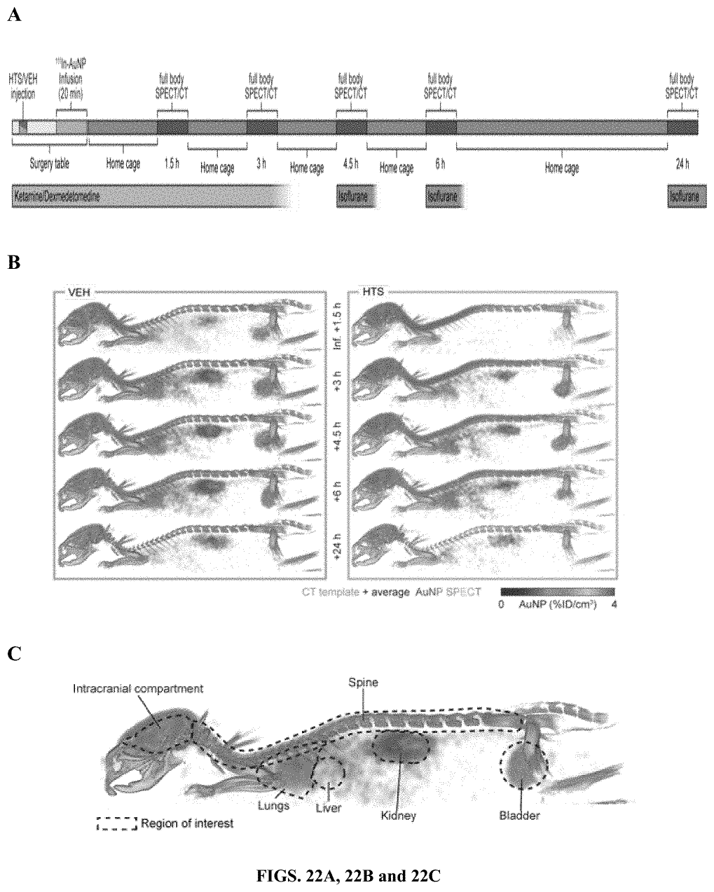

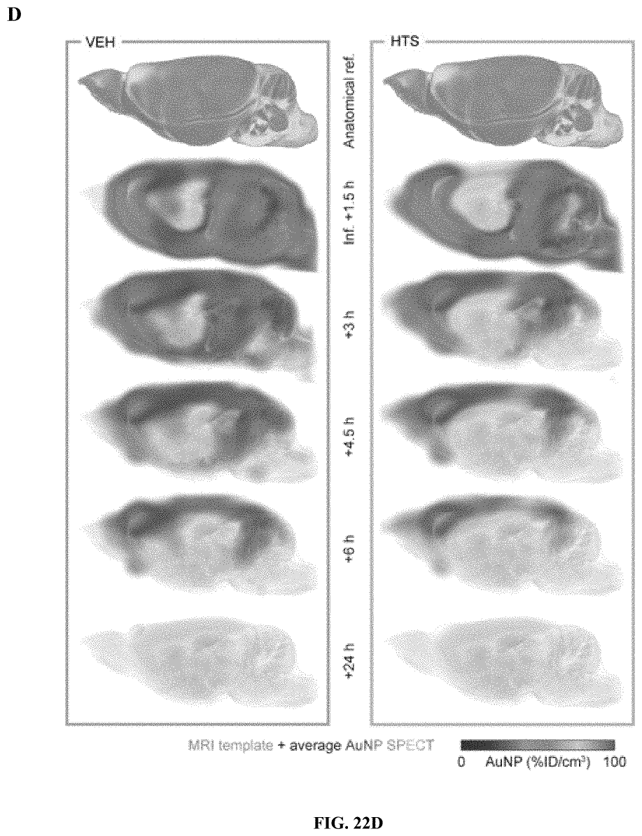

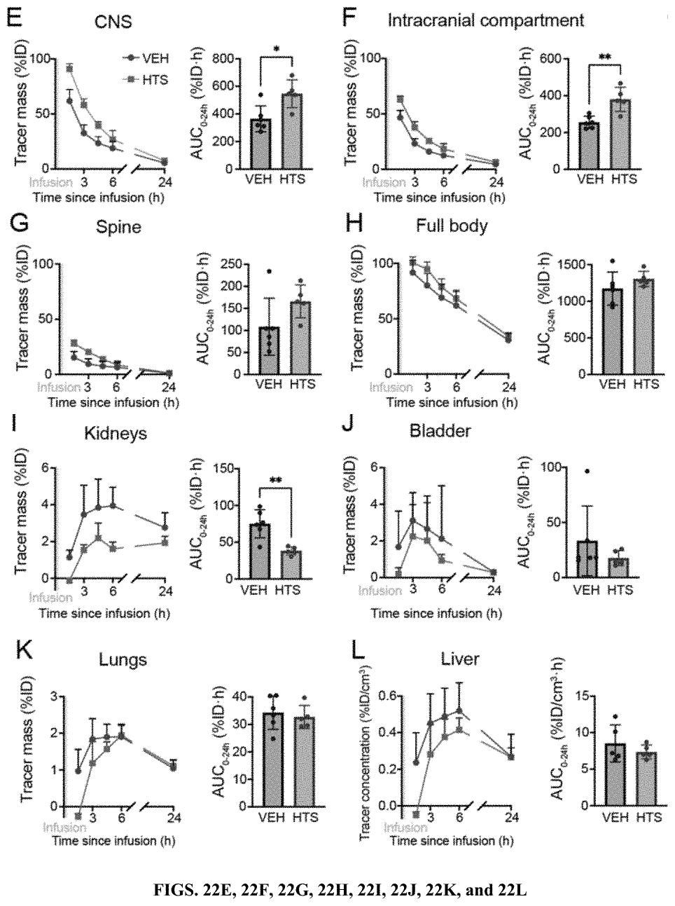

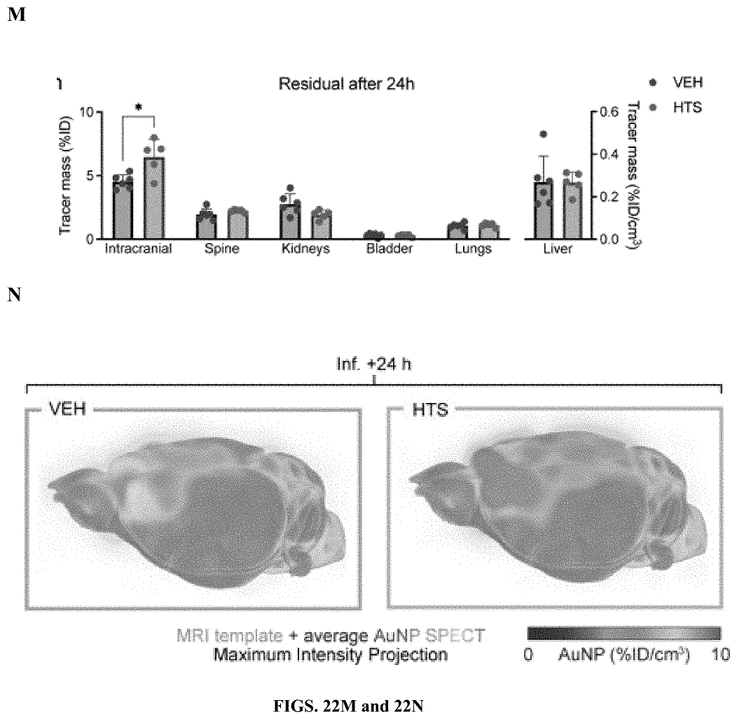

Last, the whole-body pharmacokinetics of intrathecal ¹¹¹In—AuNPs were studied over 24 hours (FIG. 22A). Rats stayed in their home cages and after recovery from initial ketamine-dexmedetomidine anesthesia they were briefly anesthetized with isoflurane for scans at 4.5, 6, and 24 hours. Gross distribution within the brain (FIG. 22B) was similar as the experiments focusing on the head and neck, with an increase in whole-CNS (FIG. 22E) and intracranial (FIG. 22C) ¹¹¹In—AuNP exposure over 24 hour (AUC₀₋₂₄ₕ) in the HTS group compared with VEH. The spinal canal (FIG. 22) or whole-body AUC₀₋₂₄ₕ did not differ between groups (FIG. 22H). Although the majority (approximately 95%) of ¹¹¹In—AuNPs had been cleared from the CNS at 24 hours, the difference in the intracranial ¹¹¹In—AuNP distribution between HTS and VEH groups remained significant (FIG. 22M). To show the distribution of residual ¹¹¹In—AuNP, inventors re-rendered the 24-hour time point from FIG. 22D with 10-fold increased contrast (FIG. 22N).

Majority of the remaining radioactivity in the brain was in close vicinity to the superior sagittal sinus and the transverse sinuses, in agreement with a previous study where accumulation of large-molecular particles near dural venous sinuses after intrathecal administration was reported (Louveau, A. et al. Nature Neuroscience 21, 1380-1391 (2018). The decay-corrected sagittal (FIG. 22H) distribution profiles over 24 hours showed relatively fast ¹¹¹In—AuNP clearance from the whole body (FIG. 22G-H). To assess the biodistribution and elimination of ¹¹¹In—AuNPs, inventors analysed their time-activity in the kidney (FIG. 22J), bladder (FIG. 22K), lungs (FIG. 22I), and liver (FIG. 22M). Relatively high activity in the kidney and bladder regions revealed renal excretion as a fast elimination route for ¹¹¹In—AuNPs. HTS significantly reduced the 24-hour kidney exposure (AUC₀₋₂₄ₕ), suggesting that clearance of ¹¹¹In—AuNPs from the CNS to general circulation was reduced by HTS. The lungs (FIG. 22L) and liver (FIG. 22M) showed only minor ¹¹¹In—AuNP exposure. In conclusion, clearance of intrathecal small ¹¹¹In—AuNPs from the whole body was rapid, with 69% and 66% radioactivity cleared from the full body at 24 hours in the VEH and HTS groups, respectively (p=0.28).

### Example 12 Stability of ¹¹¹In-LA-DOTA in Biological Media

To assess the stability of the ¹¹¹In-LA-DOTA complex when exposed to biological medium, the following mixtures were prepared: (1) CSF (30 μL)+¹¹¹In-LA-DOTA complex solution in ISO-HEPES buffer (30 μL), (2) Brain homogenate (15 μL)+PBS (15 μL)+¹¹¹In-LA-DOTA complex solution in ISO-HEPES buffer (30 μL). 1% antibiotic antimycotic solution (100×), stabilized with 10,000 units penicillin, 10 mg streptomycin and 25 μg amphotericin B per mL was added to each vial. The mixtures were incubated at 37° C. and samples were analyzed by radio-TLC at 0, 4, 24, and 48 hours. The experiment was performed in triplicate.

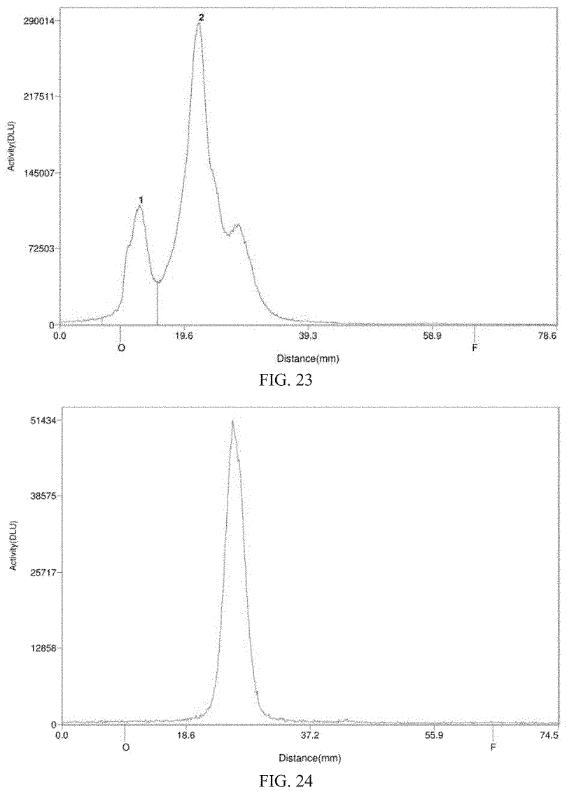

Analysis of ¹¹¹In-LA-DOTA by radio-TLC showed several peaks (FIG. 23), illustrated by a comparison to the ¹¹¹In-DOTA complex (FIG. 24). This was attributed to the lipoic acid moiety, in which the disulphide group can be expected to form various species in solution. The peak present close to the starting line was demonstrated to not correspond to any non-chelated indium, as addition of additional LA-DOTA did not change the chromatogram pattern. The same general pattern was observed for all chromatograms involving the ¹¹¹In-LA-DOTA complexes, with or without the presence of biological components.

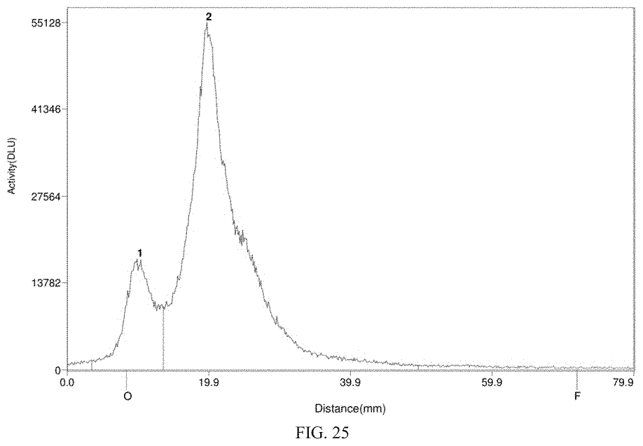

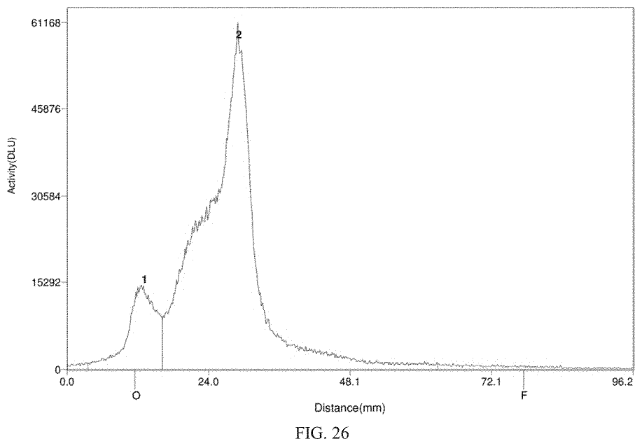

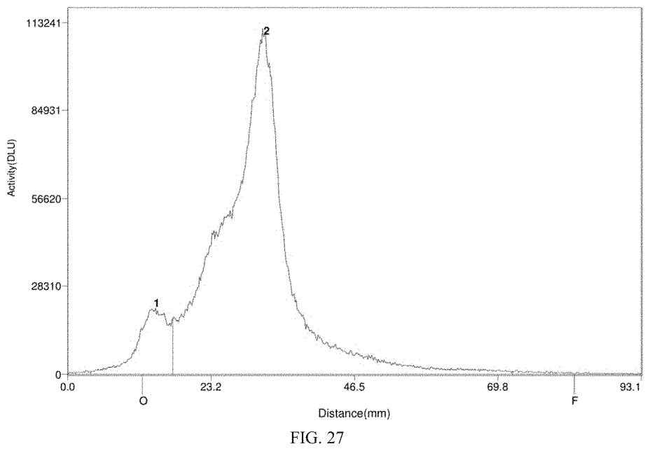

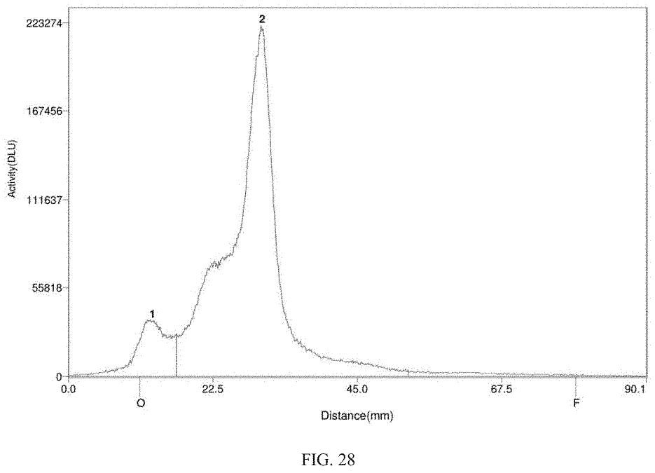

Incubation of ¹¹¹In-LA-DOTA with biological media did not result in any significant change in this pattern. In the case of dechelation of ¹¹¹In, the peak at the starting line would be expected to grow, as neither free ¹¹¹In³⁺ nor ¹¹¹In associated with biological molecules would run under the employed conditions. On this basis it was concluded that incubation of ¹¹¹In-LA-DOTA with CSF and BH did not result in appreciable destabilization of the chelate, which also corresponds with reported studies. Exemplary chromatograms for ¹¹¹In-LA-DOTA in CSF are shown in FIGS. 23-28.

Table 1 below shows area (% of total) of the first peak (closest to the starting line) observed on radio-TLC of the ¹¹¹In-LA-DOTA complex incubated in CSF or BH. The t=0 values were obtained by spotting the mixtures on the plate right after mixing. In the case of the pure ¹¹¹In-LA-DOTA complex (see FIG. 23), the first peak corresponded to 18.5% of the total.

TABLE 1

| % | CSF1 | CSF2 | CSF3 | BH1 | BH2 | BH3 |
|---|---|---|---|---|---|---|
| T = 0 | 17.2 | 16.6 | 16.1 | 16.0 | 16.4 | 17.3 |
| 4 h | 13.3 | 12.8 | 13.1 | 14.8 | 14.8 | 15.2 |
| 24 h | 11.9 | 9.4 | 9.0 | 11.4 | 11.4 | 12.2 |
| 48 h | 10.7 | 10.9 | 10.3 | 10.5 | 10.5 | 12.9 |

### Example 13 Stability of ¹¹¹In—AuNPs in Biological Media

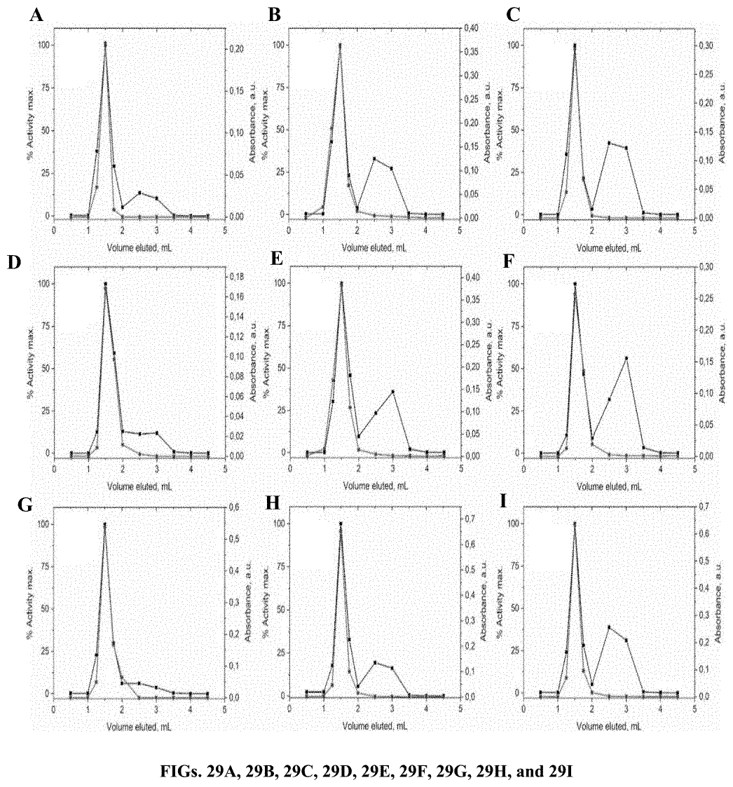

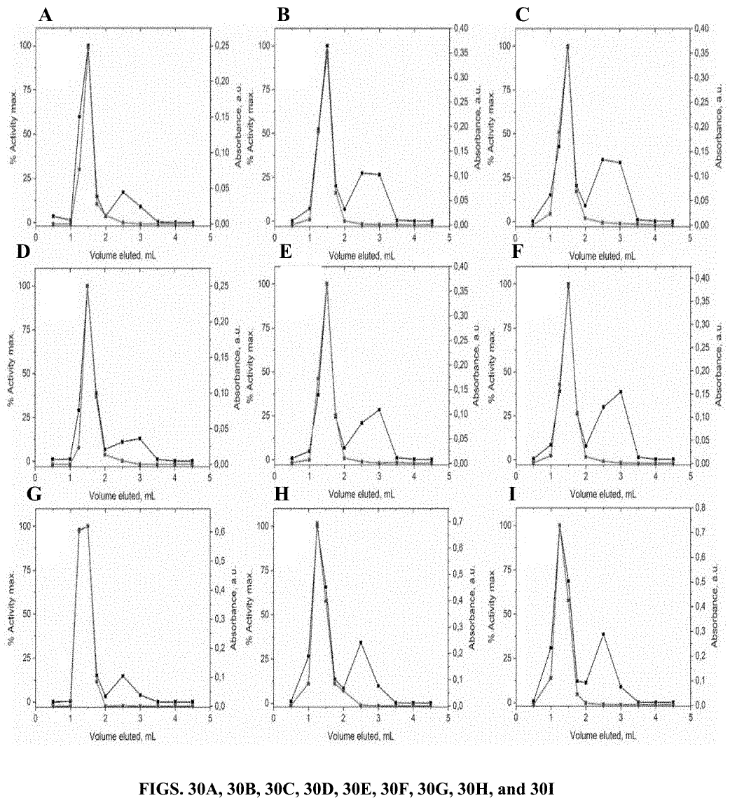

Stability of ¹¹¹In—AuNPs in biological media was examined in the same manner as described above in Example 12. Briefly, In-LA-DOTA labelled AuNPs were incubated in cerebrospinal fluid (CSF) or rat brain homogenate (BH) and analysed by size-exclusion chromatography in Sephacryl S300 HR containing cartridges. The results are shown in Table 2 below and FIGS. 29 and 30. Table 2 shows ¹¹¹In-LA-DOTA complex released from the AuNPs at various time-points (percentage of radioactivity dissociated from the AuNPs and eluting in the small-molecular fraction). Filtration results for nanoparticles incubated at 37° C. in CSF, graphs of the filtrations are displayed in FIG. 30 (CSF) and FIG. 30 (BH).

TABLE 2

| % | CSF1 | CSF2 | CSF3 | Average |
|---|---|---|---|---|
| 4 h | 14.71 | 17.31 | 5.73 | 12.59 ± 6.08 |
| 24 h | 25.90 | 24.00 | 18.02 | 22.64 ± 4.11 |
| 48 h | 33.50 | 34.10 | 30.47 | 32.69 ± 1.95 |
| | BH1 | BH2 | BH3 | Average |
| 4 h | 12.46 | 11.83 | 7.94 | 10.74 ± 2.44 |
| 24 h | 22.30 | 22.00 | 16.94 | 20.41 ± 3.01 |
| 48 h | 26.70 | 27.20 | 21.60 | 12.17 ± 3.10 |

TABLE 3

| | | | AUC_{0-3 h} (% ID · h) | | | Peak tracer mass (% ID) | | |
|---|---|---|---|---|---|---|---|---|
| Injection | Tracer | n | Intracranial | Brain | CSF | Intracranial | Brain | CSF |
| VEH | AuNP | 8 | 105.7 ± 19.2 | 60 ± 12.1 | 45.73 ± 7.2 | 62.8 ± 8.9 | 33.9 ± 6.6 | 29.7 ± 2.7 |
| HTS | AuNP | 6 | 148.7 ± 15.3^{a} | 92.9 ± 11.2^{b} | 55.79 ± 5.3^{d} | 65.6 ± 3.5 | 40.4 ± 3.2 | 29.5 ± 1.2 |
| VEH | LA-DOTA | 5 | 124.4 ± 22.5 | 84.2 ± 13.6^{c} | 40.2 ± 9.4 | 65.4 ± 6.0 | 39.6 ± 4.3 | 27.3 ± 3.5 |

| | Residual after 3 h (% ID) | | | AUC_{0-3 h} (% ID/cm³ · h) | | Peak tracer mass (% ID/cm³) | |
|---|---|---|---|---|---|---|---|
| Injection | Intracranial | Brain | CSF | Striatum | Thalamus | Striatum | Thalamus |
| VEH | 16.7 ± 4.7 | 11.0 ± 3.2 | 5.7 ± 1.5 | 2.9 ± 1.0 | 0.4 ± 0.3 | 2.8 ± 0.9 | 1.0 ± 0.6 |
| HTS | 34.1 ± 9.3^{e} | 24.3 ± 6.7^{f} | 9.8 ± 2.8^{h} | 10.9 ± 3.7ⁱ | 5.1 ± 3.2^{j} | 8.1 ± 3.0^{l} | 5.0 ± 2.9^{m} |
| VEH | 25.5 ± 6.2 | 20.6 ± 5.0^{g} | 4.9 ± 1.3 | 4.5 ± 1.5 | 7.5 ± 4.2^{k} | 4.3 ± 1.6 | 8.3 ± 3.1ⁿ |

Statistics relating to FIG. 19 in the main body. Superscript letters denote significant difference (p < 0.05) to VEH-AuNP group, one-way ANOVA.
^{a}p = 0.0014,
^{b}p = 0.0003,
^{c}p = 0.0060,
^{d}p = 0.0403,
^{e}p = 0.0004,
^{f}p = 0.0003,
^{g}p = 0.0075,
^{h}p = 0.0025,
ⁱp < 0.0001,
^{j}p = 0.0123,
^{k}p = 0.0007,
^{l}p = 0.0003,
^{m}p = 0.0097,
ⁿp < 0.0001

TABLE 4

| | | | AUC_{0-3 h} (% ID/cm³ · h) | | | |
|---|---|---|---|---|---|---|
| | | | Cervical | Nasal | Pharyngeal | Deep lymph |
| Injection | Tracer | n | spine | turbinates | lymph vessel | nodes |
| VEH | AuNP | 8 | 135.2 ± 38.14 | 7.8 ± 0.8 | 2.2 ± 0.7 | 7.2 ± 2.4 |
| HTS | AuNP | 6 | 107.3 ± 37.17 | 2.6 ± 1.9^{a} | 0.6 ± 0.2^{c} | 2.2 ± 2.0^{e} |
| VEH | LA-DOTA | 5 | 126.5 ± 30.32 | 2.4 ± 1.4^{b} | 0.5 ± 0.1^{d} | 0.1 ± 0.1^{f} |

Statistics relating to FIG. 20 in the main body. Superscript letters denote significant difference (p < 0.05) to VEH-AuNP group, one-way ANOVA.
^{a}p < 0.0001,
^{b}p < 0.0001,
^{c}p < 0.0001,
^{d}p < 0.0001,
^{e}p = 0.0004,
^{f}p < 0.0001.

TABLE 5

| | | AUC_{0-24 h} (% ID · h) | | | | | | |
|---|---|---|---|---|---|---|---|---|
| | | | Intranial | | | | | |
| Injection | n | CNS | compartment | Spine | Full body | Kidneys | Bladder | Lungs |
| VEH | 6 | 364.4 ± 94.2 | 255.9 ± 32.8 | 108.4 ± 64.8 | 1173 ± 225.4 | 75.0 ± 19.1 | 33.4 ± 31.6 | 34.3 ± 6.1 |
| HTS | 5 | 546.4 ± 100.9^{a} | 380.5 ± 65.8^{b} | 165.9 ± 37.1 | 1305 ± 104.1 | 38.21 ± 5.5^{c} | 17.6 ± 6.8 | 32.7 ± 4.2 |

| | | Residual after 24 h (% ID) | | | | | |
|---|---|---|---|---|---|---|---|
| | (% ID/cm³ · h) | Intranial | | | | | (% ID/cm³) |
| Injection | Liver | compartment | Spine | Kidneys | Bladder | Lungs | Liver |
| VEH | 8.5 ± 2.5 | 4.5 ± 0.5 | 1.9 ± 0.4 | 2.8 ± 0.8 | 0.3 ± 0.1 | 1.0 ± 0.2 | 0.3 ± 0.1 |
| HTS | 7.4 ± 0.9 | 6.5 ± 1.4^{d} | 2.2 ± 0.1 | 1.9 ± 0.4 | 0.3 ± 0.1 | 1.1 ± 0.2 | 0.3 ± 0.1 |

Statistics relating to FIG. 22 in the main body. Superscript letters denote significant difference between VEH and HTS groups (p < 0.05), unpaired t-test.
^{a}p = 0.0129,
^{b}p = 0.0027,
^{c}p = 0.0026,
^{d}p = 0.0112

### REFERENCES

1. Sevigny J et al. The antibody aducanumab reduces Abeta plaques in Alzheimer's disease. Nature. 2016;537(7618):50-6.
2. Schenk D B et al. First-in-human assessment of PRX002, an anti-alpha-synuclein monoclonal antibody, in healthy volunteers. Mov Disord. 2017;32(2):211-218.
3. Gros-Louis F, Soucy G, Lariviere R, Julien J P. Intracerebroventricular infusion of monoclonal antibody or its derived Fab fragment against misfolded forms of SOD1 mutant delays mortality in a mouse model of ALS. J Neurochem. 2010;113(5):1188-99.
4. Gallardo G, Holtzman D M. Antibody Therapeutics Targeting Abeta and Tau. Cold Spring Harb Perspect Med. 2017; 7(10)
5. Sampson J H, Maus M V, June C H. Immunotherapy for Brain Tumors. J Clin Oncol. 2017; 35(21):2450-2456.
6. Selkoe D J, Hardy J. The amyloid hypothesis of Alzheimer's disease at 25 years. EMBO Mol Med. 2016; 8(6): 595-608.
7. Klyubin I et al. Amyloid beta protein immunotherapy neutralizes Abeta oligomers that disrupt synaptic plasticity in vivo. Nat Med. 2005; 11(5):556-61.
8. Salloway S et al. Two phase 3 trials of bapineuzumab in mild-to-moderate Alzheimer's disease. N Engl J Med. 2014; 370(4):322-33.
9. Doody R S et al. Phase 3 trials of solanezumab for mild-to-moderate Alzheimer's disease. N Engl J Med. 2014; 370(4):311-21.
10. Honig LS et al. Trial of Solanezumab for Mild Dementia Due to Alzheimer's Disease. N Engl J Med. 2018; 378(4):321-330.
11. Vandenberghe R et al. Bapineuzumab for mild to moderate Alzheimer's disease in two global, randomized, phase 3 trials. Alzheimers Res Ther. 2016; 8(1):18.
12. Brody D L, Holtzman D M. Active and passive immunotherapy for neurodegenerative disorders. Annu Rev Neurosci. 2008; 31:175-93.
13. Calias P, Banks W A, Begley D, Scarpa M, Dickson P. Intrathecal delivery of protein therapeutics to the brain: a critical reassessment. Pharmacol Ther. 2014; 144(2):114-22.
14. Cohen-Pfeffer J L et al. Intracerebroventricular Delivery as a Safe, Long-Term Route of Drug Administration. Pediatr Neurol. 2017; 67:23-35.
15. Prins N D, Scheltens P. Treating Alzheimer's disease with monoclonal antibodies: current status and outlook for the future. Alzheimers Res Ther. 2013; 5(6):56.
16. Banks W A, Terrell B, Farr S A, Robinson S M, Nonaka N, Morley J E. Passage of amyloid beta protein antibody across the blood-brain barrier in a mouse model of Alzheimer's disease. Peptides. 2002; 23(12):2223-6.
17. Banks W A, Farr S A, Morley J E, Wolf K M, Geylis V, Steinitz M. Anti-amyloid beta protein antibody passage across the blood-brain barrier in the SAMP8 mouse model of Alzheimer's disease: an age-related selective uptake with reversal of learning impairment. Exp Neurol. 2007; 206(2):248-56.
18. Salloway S, Sperling R, Brashear H R. Phase 3 trials of solanezumab and bapineuzumab for Alzheimer's disease. N Engl J Med. 2014; 370(15):1460.
19. Thakker D R et al. Intracerebroventricular amyloid-beta antibodies reduce cerebral amyloid angiopathy and associated micro-hemorrhages in aged Tg2576 mice. Proc Natl Acad Sci USA. 2009; 106(11):4501-6.
20. Sperling R A et al. Amyloid-related imaging abnormalities in amyloid-modifying therapeutic trials: recommendations from the Alzheimer's Association Research Roundtable Workgroup. Alzheimers Dement. 2011; 7(4):367-85.
21. Iliff J J et al. A paravascular pathway facilitates CSF flow through the brain parenchyma and the clearance of interstitial solutes, including amyloid beta. Sci Transl Med. 2012; 4(147):147ra111.
22. Iliff J J et al. Brain-wide pathway for waste clearance captured by contrast-enhanced MRI. J Clin Invest. 2013; 123 (3): 1299-309.
23. Jessen N A, Munk A S, Lundgaard I, Nedergaard M. The Glymphatic System: A Beginner's Guide. Neurochem Res. 2015; 40(12):2583-99.
24. Rennels M L, Gregory T F, Blaumanis O R, Fujimoto K, Grady P A. Evidence for a ‘paravascular’ fluid circulation in the mammalian central nervous system, provided by the rapid distribution of tracer protein throughout the brain from the subarachnoid space. Brain Res. 1985; 326(1):47-63.
25. Rennels M L, Blaumanis O R, Grady P A. Rapid solute transport throughout the brain via paravascular fluid pathways. Adv Neurol. 1990; 52:431-9.
26. Wolak D J, Pizzo M E, Thorne R G. Probing the extracellular diffusion of antibodies in brain using in vivo integrative optical imaging and ex vivo fluorescence imaging. J Control Release. 2015; 197:78-86.
27. Pizzo M E et al. Intrathecal antibody distribution in the rat brain: surface diffusion, perivascular transport, and osmotic enhancement of delivery. J Physiol. 2018; 596(3):445-475.
28. Xie L et al. Sleep drives metabolite clearance from the adult brain. Science. 2013; 342(6156):373-7.
29. Kress B T et al. Impairment of paravascular clearance pathways in the aging brain. Ann Neurol. 2014; 76(6):845-61.
30. Iliff J J et al. Cerebral arterial pulsation drives paravascular CSF-interstitial fluid exchange in the murine brain. J Neurosci. 2013; 33(46):18190-9.
31. Lee H et al. The Effect of Body Posture on Brain Glymphatic Transport. J Neurosci. 2015; 35(31):11034-44.
32. Pullen R G, DePasquale M, Cserr H F. Bulk flow of cerebrospinal fluid into brain in response to acute hyperosmolality. Am J Physiol. 1987; 253(3 Pt 2):F538-45.
33. Cserr H F, DePasquale M, Patlak C S. Volume regulatory influx of electrolytes from plasma to brain during acute hyperosmolality. Am J Physiol. 1987; 253(3 Pt 2):F530-7.
34. Cserr H F, DePasquale M, Nicholson C, Patlak C S, Pettigrew K D, Rice M E. Extracellular volume decreases while cell volume is maintained by ion uptake in rat brain during acute hypernatremia. J Physiol. 1991; 442:277-95.
35. Peng W et al. Suppression of glymphatic fluid transport in a mouse model of Alzheimer's disease. Neurobiol Dis. 2016; 93:215-25.
36. Yang L et al. Evaluating glymphatic pathway function utilizing clinically relevant intrathecal infusion of CSF tracer. J Transl Med. 2013; 11:107.
37. Smith A J, Yao X, Dix J A, Jin B J, Verkman A S. Test of the ‘glymphatic’ hypothesis demonstrates diffusive and aquaporin-4-independent solute transport in rodent brain parenchyma. Elife. 2017; 6.
38. Mestre H et al. Aquaporin-4 dependent glymphatic solute transport in rodent brain. bioRxiv. 2017.
39. Lundgaard I et al. Glymphatic clearance controls state-dependent changes in brain lactate concentration. J Cereb Blood Flow Metab. 2017; 37(6):2112-2124.
40. Murlidharan G, Crowther A, Reardon R A, Song J, Asokan A. Glymphatic fluid transport controls paravascular clearance of AAV vectors from the brain. J C I Insight. 2016; 1(14):e88034.
41. Jiang Q et al. Impairment of the glymphatic system after diabetes. J Cereb Blood Flow Metab. 2017; 37(4):1326-1337.
42. Rangroo Thrane V et al. Paravascular microcirculation facilitates rapid lipid transport and astrocyte signaling in the brain. Sci Rep. 2013; 3:2582.
43. Achariyar T M et al. Glymphatic distribution of CSF-derived apoE into brain is isoform specific and suppressed during sleep deprivation. Mol Neurodegener. 2016; 11(1):74.
44. Lundgaard I et al. Direct neuronal glucose uptake heralds activity-dependent increases in cerebral metabolism. Nat Commun. 2015; 6:6807.
45. Venkat P et al. White matter damage and glymphatic dysfunction in a model of vascular dementia in rats with no prior vascular pathologies. Neurobiol Aging 2017; 50:96-106.
46. Benveniste H et al. Anesthesia with Dexmedetomidine and Low-dose Isoflurane Increases Solute Transport via the Glymphatic Pathway in Rat Brain When Compared with High-dose Isoflurane. Anesthesiology. 2017; 127(6):976-988.
47. Gaberel T et al. Impaired glymphatic perfusion after strokes revealed by contrast-enhanced MRI: a new target for fibrinolysis? Stroke. 2014; 45(10):3092-6.
48. Ringstad G, Vatnehol S A S, Eide P K. Glymphatic MRI in idiopathic normal pressure hydrocephalus. Brain. 2017; 140(10):2691-2705.
49. Brain Trauma F, American Association of Neurological S, Congress of Neurological S, Joint Section on N, Critical Care AC, Bratton SL, et al. Guidelines for the management of severe traumatic brain injury. II. Hyperosmolar therapy. J Neurotrauma. 2007; 24 Suppl 1:S14-20.
50. Ma Q, Ineichen B V, Detmar M, Proulx S T. Outflow of cerebrospinal fluid is predominantly through lymphatic vessels and is reduced in aged mice. Nat Commun. 2017; 8(1):1434.
51. Bedussi B et al. Paravascular channels, cisterns, and the subarachnoid space in the rat brain: A single compartment with preferential pathways. J Cereb Blood Flow Metab. 2017; 37(4): 1374-1385.
52. Muizelaar J P, Lutz H A 3rd, Becker D P. Effect of mannitol on ICP and CBF and correlation with pressure autoregulation in severely head-injured patients. J Neurosurg. 1984; 61(4):700-6.
53. Domaingue C M, Nye D H. Hypotensive effect of mannitol administered rapidly. Anaesth Intensive Care. 1985; 13 (2): 134-6.
54. Xu Z et al. Deletion of aquaporin-4 in APP/PS1 mice exacerbates brain Abeta accumulation and memory deficits. Mol Neurodegener. 2015; 10:58.
55. Golde T E. Open questions for Alzheimer's disease immunotherapy. Alzheimers Res Ther. 2014; 6(1):3.
56. Nevins T D, Kelley D H. Front tracking for quantifying advection-reaction-diffusion. Chaos. 2017; 27(4):043105.
57. Silasi G, Xiao D, Vanni M P, Chen A C, Murphy T H. Intact skull chronic windows for mesoscopic wide-field imaging in awake mice. J Neurosci Methods. 2016; 267:141-9.
58. Demattos R B et al. A plaque-specific antibody clears existing beta-amyloid plaques in Alzheimer's disease mice. Neuron. 2012; 76(5):908-20.
59. Klunk W E et al. Imaging Abeta plaques in living transgenic mice with multiphoton microscopy and methoxy-X04, a systemically administered Congo red derivative. J Neuropathol Exp Neurol. 2002; 61(9):797-805.
60. Schneider C A, Rasband W S, Eliceiri K W. NIH Image to ImageJ: 25 years of image analysis. Nat Methods. 2012; 9(7):671-5.
61. Schindelin J et al. Fiji: an open-source platform for biological-image analysis. Nat Methods. 2012; 9(7): 676-82.
The foregoing examples and description of the preferred embodiments should be taken as illustrating, rather than as limiting the present invention as defined by the claims. As will be readily appreciated, numerous variations and combinations of the features set forth above can be utilized without departing from the present invention as set forth in the claims. Such variations are not regarded as a departure from the scope of the invention, and all such variations are intended to be included within the scope of the following claims. All references cited herein are incorporated by reference in their entireties.

What is claimed is:

1. A method for improving delivery of a composition comprising a nanoparticle to a central nervous system interstitium, brain interstitium and/or a spinal cord interstitium of a. subject comprising:
(1) enhancing glymphatic system influx; and
(2) delivering the composition to the central nervous system interstitium, brain interstitium and/or the spinal cord interstitium.

2. The method of claim 1, wherein the step of enhancing glymphatic system influx comprises pumping fluid through the central nervous system interstitium.

3. The method of claim 1, wherein the step of enhancing glymphatic system influx comprises administering an agent to the subject.

4. The method of claim 3, wherein the agent is a hypertonic solution and administered into plasma of the subject.

5. The method of claim 4, wherein the hypertonic solution comprises NaCl or Mannitol.

6. The method of claim 3, wherein the agent is a Stat-3 inhibitor, a molecule known in the art to be bone morphogenetic protein (BMP) signaling axis molecule, an antagonist of AVP (vasopressin), an antagonist of atrial natriuretic peptide (ANP), an antagonist of Angiotensin II, an antagonist of AT2R receptors, or an antagonist of AT1 receptors.

7. The method of claim 1, wherein the composition is delivered intracisternally or intrathecally.

8. The method of claim 1, wherein the composition is delivered at about the same time or after the glymphatic system influx is enhanced.

9. The method of claim 1, wherein the nanoparticle is about 10 to about 15 nm in diameter.

10. The method claim 1, wherein the nanoparticle is linked to or conjugated to or coated with or encompassing a small molecule, a polymer, a virus, a large molecule, a peptide, an antibody, a nucleic acid, or a biologically active fragment thereof.

11. A method for treating a neurological disorder in a subject, comprising
(1) enhancing glymphatic system influx; and
(2) delivering a therapeutic composition comprising a nanoparticle to the central nervous system interstitium, brain interstitium and/or the spinal cord interstitium.

12. The method of claim 11, wherein the step of enhancing glymphatic system influx comprises pumping fluid through the central nervous system interstitium.

13. The method of claim 11, wherein the step of enhancing glymphatic system influx comprises administering an agent to the subject.

14. The method of claim 13, wherein the agent is a hypertonic solution and administered into plasma of the subject.

15. The method of claim 14, wherein the hypertonic solution comprises NaCl or Mannitol.

16. The method of claim 14, wherein the agent is a Stat-3 inhibitor, a BMP signaling axis molecule, an antagonist of AVP (vasopressin), an antagonist of atrial natriuretic peptide (ANP), an antagonist of Angiotensin II, an antagonist of AT2R receptors, or an antagonist of AT1 receptors.

17. The method of claim 11, wherein the composition is delivered intracisternally or intrathecally.

18. The method of claim 11, wherein the composition is delivered at about the same time or after the glymphatic system influx is enhanced.

19. The method of claim 11, wherein the nanoparticle is about 10 to about 15 nm in diameter.

20. The method of claim 11, wherein the nanoparticie is linked to or conjugated to or coated with or encompassing a small molecule, a polymer, a virus, a large molecule, a peptide, an antibody, a nucleic acid, or a biologically active fragment thereof.

21. The method of claim 11, wherein the neurological disorder is selected from the group consisting of a neuropathy, an amyloidosis, cancer, an ocular disease or disorder, a viral or microbial infection, inflammation, ischemia, neurodegenerative disease, seizure, behavioral disorder, and lysosomal storage disease.

22. The method of claim 4, wherein the agent is a hypertonic saline and administered intravenously to the subject.

23. The method of claim 1, wherein the subject is anesthetized with a composition comprising ketamine and dexmedetomidine before the step of enhancing, the step of delivering, or both.
